SEQUENCE LISTING (7 sequences)

SEQ ID NO: 1 - DNA, 4488 bp, synthetic construct
  source 1..4488: mol_type = other DNA
gctagccaacaaagcgacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgcttgtatgggaagccccatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataatcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcactagttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcaacgcgtgagcccaccagctccgtaagttcgggtgctgtgtggctcgtacccgcgcattcaggcggcagggggtctaacgggtctaaggcggcgtgtacggccgccacagcggctcttagcggcccggaaacgtcctcgaaacgacgcatgtgttcctcctggttggtacaggtggttgggggtgctcggctgtcgctggtgtttcatcatcagggctcgacgggagagcgggggagtgtgcagttgtggggtggcccctcagcgaaatatctgacttggagctcgtgtcggaccatacaccggtgattaatcgtggtttattatcaagcgtgagccacgtcgccgacgaatttgagcagctctggctgccgtactggtccctggcaagcgacgatctgctcgaggggatctaccgccaaagccgcgcgtcggccctaggccgccggtacatcgaggcgaacccaacagcgctggcaaacctgctggtcgtggacgtagaccatccagacgcagcgctccgagcgctcagcgcccgggggtcccatccgctgcccaacgcgatcgtgggcaatcgcgccaacggccacgcacacgcagtgtgggcactcaacgcccctgttccacgcaccgaatacgcgcggcgtaagccgctcgcatacatggcggcgtgcgccgaaggccttcggcgcgccgtcgatggcgaccgcagttactcaggcctcatgaccaaaaaccccggccacatcgcctgggaaacggaatggctccactcagatctctacacactcagccacatcgaggccgagctcggcgcgaacatgccaccgccgcgctggcgtcagcagaccacgtacaaagcggctccgacgccgctagggcggaattgcgcactgttcgattccgtcaggttgtgggcctatcttcccgccctcatgcggatctacctgccgacccggaacgtggacggactcggccgcgcgatctatgccgagtgccacgcgcgaaacgccgaatttccgtgcaacgacgtgtgtcccggaccgctaccggacagcgaggtccgcgccatcgccaacagcatttggcgttggatcacaaccaagtcgcgcatttgggcggacgggatcgtggtctacgaggccacactcagtgcgcgccatgcggccatctcgcggaagggcgcagcagcgcgcacggcggcgagcacagttgcgcggcgcgcaaagtccgcgtcagccatggaggcattgctatgagcgacggctacagcgacggctacagcgacggctacaactggcagccgactgtccgcaaaaagcggcgcgtgaccgccgccgaaggcgctcgaatcaccggactatccgaacgccacgtcgtccggctcgtggcgcaggaacgcagcgagtggttcgccgagcaggctgcacgccgcgaacgcatccgcgcctatcacgacgacgagggccactcttggccgcaaacggccaaacatttcgggctgcatctggacaccgttaagcgactcggctatcgggcgaggaaagagcgtgcggcagaacaggaagcggctcaaaaggcccacaacgaagccgacaatccaccgctgttctaacgcaattggggagcgggtgtcgcgggggttccgtggggggttccgttgcaacgggtcggacaggtaaaagtcctggtagacgctagttttctggtttgggccatgcctgtctcgttgcgtgtttcgttgcgtccgttttgaataccagccagacgagacggggttctacgaatcttggtcgataccaagccatttccgctgaatatcgtggagctcaccgccagaatcggtggttgtggtgatgtacgtggcgaactccgttgtagtgcttgtggtggcatccgtggcgcggccgcggtaccagatctttaaatctagaggtgaccacaacgacgcgcccgctttgatcggggacgtctgcggccgaccatttacgggtcttgttgtcgttggcggtcatgggccgaacatactcacccggatcggagggccgaggacaaggtcgaacgaggggcatgacccggtgcggggcttcttgcactcggcataggcgagtgctaagaataacgttggcactcgcgaccggtgagtcgtaggtcgggacggtgaggccaggcccgtcgtcgcagcgagtggcagcgaggacaacttgagccgtccgtcgcgggcactgcgcccggccagcgtaagtagcggggttgccgtcacccggtgacccccggtttcatccccgatccggaggaatcacttcgatatcgccaagacaattgcggatccagctgcagaattcgaagcttatcgatgtcgacgtagttaactagcgtacgatcgactgccaggcatcaaataaaacgaaaggctcagtcgaaagactgggcctttcgttttatctgttgtttgtccggccatcatggccgcggtgatca

SEQ ID NO: 2 - DNA, 5653 bp, synthetic construct
  source 1..5653: mol_type = other DNA
aaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaggggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcaacgcgtgagcccaccagctccgtaagttcgggcgctgtgtggctcgtacccgcgcattcaggcggcagggggtctaacgggtctaaggcggcgtgtacggccgccacagcggctctcagcggcccggaaacgtcctcgaaacgacgcatgtgttcctcctggttggtacaggtggttgggggtgctcggctgtcgctggtgttccaccaccagggctcgacgggagagcgggggagtgtgcagttgtggggtggcccctcagcgaaatatctgacttggagctcgtgtcggaccatacaccggtgattaatcgtggtctactaccaagcgtgagccacgtcgccgacgaatttgagcagctctggctgccgtactggccgctggcaagcgacgatctgctcgaggggatctaccgccaaagccgcgcgtcggccctaggccgccggtacatcgaggcgaacccaacagcgctggcaaacctgctggtcgtggacgtagaccatccagacgcagcgctccgagcgctcagcgcccgggggtcccatccgctgcccaacgcgatcgtgggcaatcgcgccaacggccacgcacacgcagtgtgggcactcaacgcccctgttccacgcaccgaatacgcgcggcgtaagccgctcgcatacatggcggcgtgcgccgaaggccttcggcgcgccgtcgatggcgaccgcagttactcaggcctcatgaccaaaaaccccggccacatcgcctgggaaacggaatggctccactcagatctctacacactcagccacatcgaggccgagctcggcgcgaacatgccaccgccgcgctggcgtcagcagaccacgtacaaagcggctccgacgccgctagggcggaattgcgcactgttcgattccgtcaggttgtgggcctatcgtcccgccctcatgcggatctacctgccgacccggaacgtggacggactcggccgcgcgatctatgccgagtgccacgcgcgaaacgccgaatttccgtgcaacgacgtgtgtcccggaccgctaccggacagcgaggtccgcgccatcgccaacagcatttggcgttggatcacaaccaagtcgcgcatttgggcggacgggatcgtggtctacgaggccacactcagtgcgcgccagtcggccatctcgcggaagggcgcagcagcgcgcacggcggcgagcacagttgcgcggcgcgcaaagtccgcgtcagccatggaggcattgctatgagcgacggctacagcgacggctacaaccggcagccgactgtccgcaaaaagcggcgcgtgaccgccgccgaaggcgctcgaatcaccggactatccgaacgccacgtcgtccggctcgtggcgcaggaacgcagcgagtggctcgccgagcaggctgcacgccgcgaacgcatccgcgcctatcacgacgacgagggccactcttggccgcaaacggccaaacatttcgggctgcatctggacaccgttaagcgactcggctatcgggcgaggaaagagcgtgcggcagaacaggaagcggctcaaaaggcccacaacgaagccgacaatccaccgctgttctaacgcaattggggagcgggtgtcgcgggggttccgtggggggttccgttgcaacgggtcggacaggtaaaagtcctggtagacgctagttttctggtttgggccatgcctgtctcgttgcgtgtttcgttgcgcccgttttgaataccagccagacgagacggggttctacgaatcttggtcgataccaagccatttccgctgaatatcggggagctcaccgccagaatcggtggttgtggtgatgtacgtggcgaactccgttgtagtgcctgtggtggcatccgtggcgcggccgcggtaccagatctttaaatctagacggtgaccacaacgcgcccgctttgatcggggacgtctgcggccgaccatttacgggtcttgttgtcgttggcggtcatgggccgaacatactcacccggatcggagggccgaggacaaggtcgaacgaggggcatgacccggtgcggggcttcttgcactcggcataggcgagtgctaagaataacgttggcactcgcgaccggtgagtgctaggtcgggacggtgaggccaggcccgtcgtcgcagcgagtggcagcgaggacaacttgagccgtccgtcgcgggcactgcgcccggccagcgtaagtagcggggttgccgtcacccggtgacccccgtttcatccccgatccggaggaatcacttcgatatcgccaagacaattgcggatcatgacaatgcgaatagatacagacaaacaaatgaatttacttagtgataagaacgttgcaataattggtggtggacccgttggactgactatggcaaaattattacagcaaaacggcatagacgtttcagtttacgaaagagacaacgaccgagaggcaagaatttttggtggaacccttgacctacacaaaggttcaggtcaggaagcaatgaaaaaagcgggattgttacaaacttattatgacttagccttaccaatgggtgtaaatattgctgataaaaaaggcaatattttatccacaaaaaatgtaaagcccgaaaatcgatttgacaatcctgaaataaacagaaatgacttaagggctatcttgttgaatagtttagaaaacgacacggttatttgggatagaaaacttgttatgcttgaacctggtaagaagaagtggacactaacttttgagaataaaccgagtgaaacagcagatttggttattcttgccaatggcgggatgtccaaggtaagaaaatttgttaccgacacggaagttgaagaaacaggtactttcaatatacaagccgatattcatcaaccagagataaactgtcctggattttttcagctatgcaatggaaaccggctaatggcatctcaccaaggtaatttattatttgctaaccccaataataatggtgcattgcattttggaataagttttaaaacacctgatgaatggaaaaaccaaacgcaggtagattttcaaaacagaaatagtgtcgttgattttcttctgaaagaattttccgattgggacgaacgctacaaagaattgattcatacgacgttgtcatttgtaggattggctacacggatatttcctttagaaaagccttggaaaagcaagcgcccattacccataacaatgattggggatgccgcacatttgatgccgccttttgcagggcagggagtaaatagtgggttggtggatgccttgatattgtctgataatctagccgatggaaaatttaatagcattgaagaggctgttaaaaattatgaacagcaaatgtttatctatggcaaagaagcacaagaagaatcaactcaaaacgaaattgaaatgtttaaacccgactttacgtttcagcaattgttaaatgtatacccatacgatgttccagattacgcttaaagcttatcgatgtcgacgtagttaactagcgtacgatcgactgccaggcatcaaataaaacgaaaggctcagtcgaaagactgggcctttcgttttatgccatcatggccgcggtgatcagctagccacctgacgtcggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgcttgtatgggaagccccatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataatcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcaccaaccgtggctccctcactttctggctggatgatggggcgattcaggcctggtatgagtcagcaacaccttcttcacgaggcagacctcactagttccatgagcgtcagaccccgtagaaaagatcaaaggatcttcttgagatcctttttttctgcgcgtaatctgctgcttgcaaac

SEQ ID NO: 3 - DNA, 2598 bp, synthetic construct
  source 1..2598: mol_type = other DNA
ccaaccttaccagagggcgccccagctggcaattccgacgtctgtgtggaattgtgagcggataacaaattcacacagggccctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaactgtcgactcgagcatagcatttttatccataagattagcggatcctaagctttacaattgtgagcgctcacaattatgatagattcaattgtgagcggataacaatttcacacagaattcattaaagaggagaaaggtaccgggccccccctcgaggtcgacggtatcgataagcttgatatcgaattcctgcagcccgggggatcccatggtacgcgtgctagaggcatcaaataaaacgaaaggctcagtcgaaagactgggcctttcgttttatctgttgtttgtcggtgaacgctctcctgagtaggacaaatccgccgccctagacctagggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttc

SEQ ID NO: 4 - DNA, 3675 bp, synthetic construct
  source 1..3675: mol_type = other DNA
ggatcccatggtacgcgtgctagaggcatcaaataaaacgaaaggctcagtcgaaagactgggcctttcgttttatctgttgtttgtcggtgaacgctctcctgagtaggacaaatccgccgccctagacctagggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccgacgtctgtgtggaattgtgagcggataacaaattcacacagggccctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaactgtcgactcgagcatagcatttttatccataagattagcggatcctaagctttacaattgtgagcgctcacaattatgatagattcaattgtgagcggataacaatttcacacagaattcattaaagaggagaaaggtaccatgactttgctaaaaaataaaaaaattacaataattggtgccgggcctgttggattaacgatggcgagattgttacagcaaaaccgcgtggacgttacagtttacgagagagacaaagaccgagatgcaaggatttttggtgggacacttgacctgcacagggattcgggacaggaagcaatgaaaagagcgggattgttacaaacttattatgacttagctttaccaatgggtgtaaatattgctgatgaaaagggtaatattttaaccacaaaaaatgtaaagcccgaaaatcggtttgacaatcctgaaataaacagaaatgacttaagaactatcttattaaatagcctacaaaatgacaccgtcatttgggatagaaaacttgttacgcttgaacctgataaggagaagtggacactaacttttgaagataaaccgagtgaaacagcagatctggttattattgccaatggtggaatgtctaaagtaagaaaatttgttaccgacacggaagttgaagaaacaggtactttcaatatacaagccgatattcatcaaccagaggtgaactgtcctggattttttcagctatgcaatggaaaccggctaatggctgctcatcaaggtaatttattatttgcgaatcctaataataatggtgcattgcattttggaataagttttaaaacatctgatgaatggaaaagcaagactctggtagattttcaagacagaaatagtgtcgttgattttctcctgaaaaaattttccgattgggatgaacgctacaaagaactgattcgtgtgacatcatcttttgtagggttagcgacacgaatatttcccttaggtaagtcttggaaaagtaagcgtccattacccataacgatgattggagatgctgctcatttgatgcctccttttgcaggacaaggcgtaaacagcgggttgatggatgccttgatattgtcggataatctgaccaatgggaaatttaacagcattgaagaggctattgaaaattatgaacagcaaatgtttgcttatggaagagaagcacaggcagaatcaataataaacgaaacggaaatgttcagttccgacttttcattccaaaaactaatgaatctataa

SEQ ID NO: 5 - DNA, 3705 bp, synthetic construct
  source 1..3705: mol_type = other DNA
ggatcccatggtacgcgtgctagaggcatcaaataaaacgaaaggctcagtcgaaagactgggcctttcgttttatctgttgtttgtcggtgaacgctctcctgagtaggacaaatccgccgccctagacctagggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccgacgtctgtgtggaattgtgagcggataacaaattcacacagggccctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaactgtcgactcgagcatagcatttttatccataagattagcggatcctaagctttacaattgtgagcgctcacaattatgatagattcaattgtgagcggataacaatttcacacagaattcattaaagaggagaaaggtaccatgactaaacatataaaaattcttgtaataggggttggggttgcgggccctgcagtcgcttattggttaaaaagatttgggttttctccggtcttaattgaaaaatcagctgccgttagaaaagggggccaggcacttgatattcgcggcatagccacacatatcgctaaagaaatgggcatttacgaccagatatgtaacatgcggactcaaataaagtgtggacgttatgtggatgtaaagggtaatgttttgcatgaagaacaaggtgaaacctttggtttccggcaagatgacgaagttgagattcttcgtggagacctagttgaaattctaatgaaagcaattgcagatataccttgtgaattcaaacaatccgtcataaaaattgaacaaaacgaagatagtgtaactgtcacctacaaagatggcagagttgaaaattatgaccttgtgatcgctgccgatggtattcattctgctaccaggggaatggtttttagtaaaaatgaatatcagttgattaatctcggctcctacgtcagcgcatttaccatcccaaattatctcggtttggatcacatggaactcctatgtgaatcaaatcataaactggttactctacaaagcgacagccaagctgacaaagccatggcgggatttatgttccgttcaaaacatgtattagaagatattcgtgatgagcaagaacaaaaacatttcttacacgccagttttcaaaatttcggttgggaaacacaaaatattttaaatcgaatgccagaaagcgatgacttttattttgacgcgattactcaaatcaaaatgaaaagctggactaaaggccgtatagccttaatcggcgatgccgcttattgcccctcacctttatcaggtcaggggaataatttagcttttgttggcgcttatattttggcgggtgaattgaaaaaggctgatggtgattatatacaagcatttacccgatataacgaattgctacatccctttgttgaggctaaccaacaatttggtgtgtgggtaagcgaatcctttctcctaaaagatgatgaagtctctaaagagattgcggaagcccgttctaataaaattttggcaatgataaagtcagtctctaattcaataaacttgccacaatacgaataa

SEQ ID NO: 6 - DNA, 39 bp, synthetic construct
  source 1..39: mol_type = other DNA
agacaattgcggatcatgacaatgcgaatagatacagac

SEQ ID NO: 7 - DNA, 61 bp, synthetic construct
  source 1..61: mol_type = other DNA
cgacatcgataagctttaagcgtaatctggaacatcgtatgggtatacatttaacaattgc

(19) United States
(12) Patent Application Publication
(10) Pub. No.: US 2026/0060992 A1
(43) Pub. Date: Mar. 5, 2026

(54) COMPOSITIONS FOR OVERCOMING TETRACYCLINE ANTIBIOTIC RESISTANCE AND METHODS OF USE THEREOF

(71) Applicants: Washington University, St. Louis, MO (US); The United States of America, as Represented by the Secretary, Department of Health and Human Servic, Bethesda, MD (US)

(72) Inventors: Timothy Wencewicz, St. Louis, MO (US); Gautam Dantas, St. Louis, MO (US); Niraj Tolia, Bethesda, MD (US); Emily Williford, St. Louis, MO (US); Kevin Blake, St. Louis, MO (US); Yao-Peng Xue, St. Louis, MO (US); Wai Kwan Tang, Bethesda, MD (US)

(73) Assignees: Washington University, St. Louis, MO (US); The United States of America, as Represented by the Secretary, Department of Health and Human Servic, Bethesda, MD (US)

(21) Appl. No.: 19/313,798

(22) Filed: Aug. 28, 2025

Related U.S. Application Data
(60) Provisional application No. 63/687,889, filed on Aug. 28, 2024.

(51) Int. Cl. A61K 31/65; A61P 31/04; C12N 9/99

(52) U.S. Cl. CPC A61K 31/65; A61P 31/04; C12N 9/99

(57) ABSTRACT

Compositions for overcoming tetracycline antibiotic resistance and methods of use thereof are provided. Compositions include at least one compound selected from Formula (I) and Formula (II) as disclosed herein. Methods of treating a subject having a bacterial infection include administering at least one compound selected from Formula (I) and Formula (II) as disclosed herein. In some embodiments, the composition further includes at least one tetracycline antibiotic.

### CROSS-REFERENCE TO RELATED APPLICATIONS

This application claims the benefit of priority to U.S. Provisional Application Ser. No. 63/687,889 filed 28 Aug. 2024, which is incorporated herein by reference in its entirety.

### STATEMENT OF FEDERALLY SPONSORED RESEARCH OR DEVELOPMENT

This invention was made with government support under AI123394 awarded by the National Institutes of Health and under DGE2139839 awarded by the National Science Foundation. The government has certain rights in the invention.

### MATERIAL INCORPORATED BY REFERENCE

The Sequence Listing, which is a part of the present disclosure, includes a computer-readable form comprising nucleotide and/or amino acid sequences of the present invention (file name “021043-US-NP_2025-08-28_Sequence-Listing” created on 21 Aug. 2025; 27,209 bytes). The subject matter of the Sequence Listing is incorporated herein by reference in its entirety.

### FIELD

The present disclosure generally relates to development of antibiotics and treatments for infectious diseases.

### BACKGROUND

Tetracycline antibiotic resistance poses challenges in clinical, agricultural, and other applicable settings. Tetracyclines (TCs) are essential antibacterial agents used for treating a wide range of clinical infections caused by aerobic, anaerobic, Gram-positive, and Gram-negative pathogens. Third generation drugs including tigecycline (Tig), eravacycline, and omadacycline are considered drugs of last resort for treating multi-drug resistant (MDR) 30 pathogens such as MDR Acinetobacter baumannii. These third-generation drugs overcome traditional clinical resistance mechanisms including efflux pumps and ribosome protection proteins, but a new resistance mechanism is emerging in the form of tetracycline-inactivating enzymes known as tetracycline destructases (TDases). TDases have been found within inducible antibiotic resistance operons in MDR bacterial pathogens including A. baumannii, Pseudomonas aeruginosa, Legionella longbeacha, and Mycobacteria abscessus. TDases can inactivate all generations of the TC antibiotic family and threaten the clinical viability of tetracycline drugs of last resort.

### BRIEF DESCRIPTION OF THE DISCLOSURE

Among the various aspects of the present disclosure are the provisions of overcoming antibiotic resistance with c10-ester derivatives of anhydrotetracycline, and particularly in combination with tetracycline antibiotics.

In accordance with an aspect of the present disclosure, a composition is provided. The composition comprises at least one compound according to

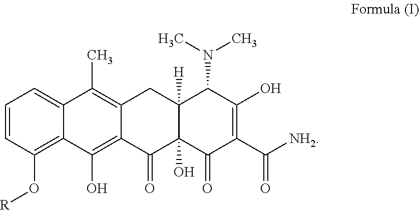

In some embodiments, R is selected from:

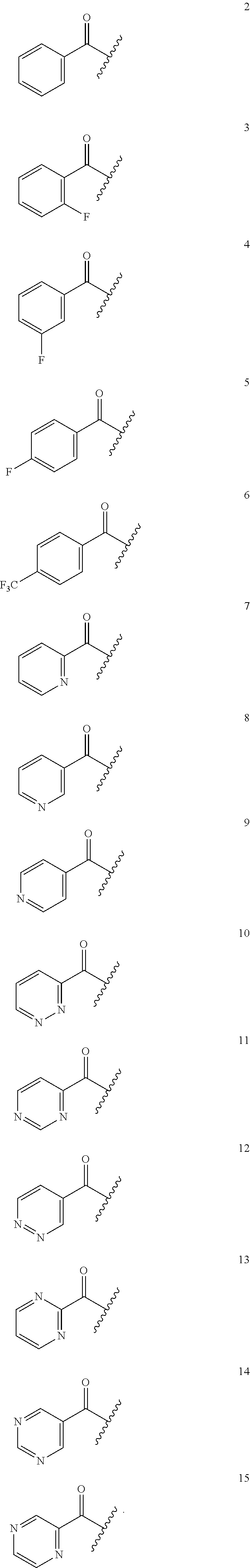

In some embodiments, the composition further comprises a tetracycline antibiotic.

In accordance with another aspect of the present disclosure, a composition is provided. The composition comprises at least one compound according to

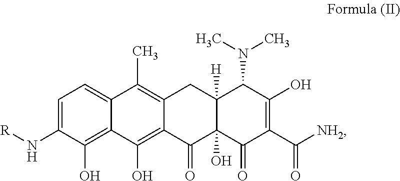

In some embodiments, R is selected from

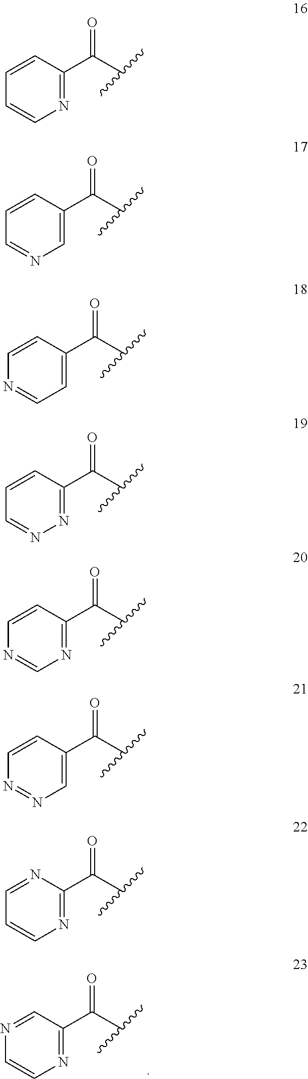

In some embodiments, the composition further comprises a tetracycline antibiotic.

In accordance with another aspect of the present disclosure, a method of treating a subject having a bacterial infection is provided. The method comprises administering to the subject a therapeutically effective amount of a composition comprising at least one compound selected from a C10-benzoate and a C9-benzamide.

In some embodiments, the C10-benzoate comprises

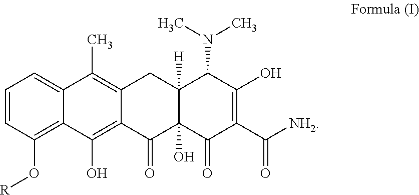

In some embodiments, R of Formula (I) is selected from:

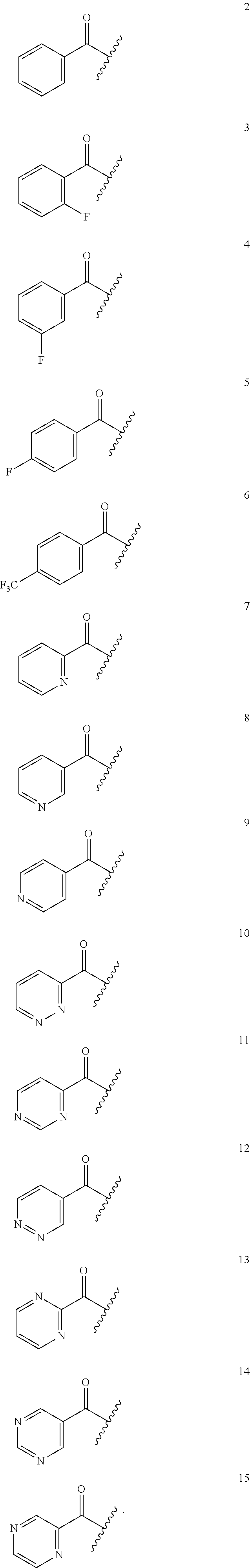

In some embodiments, the C9-benzide comprises

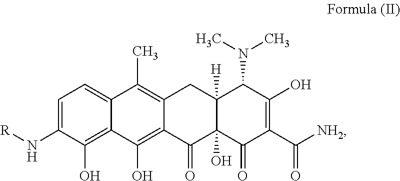

In some embodiments, R of Formula (II) is selected from:

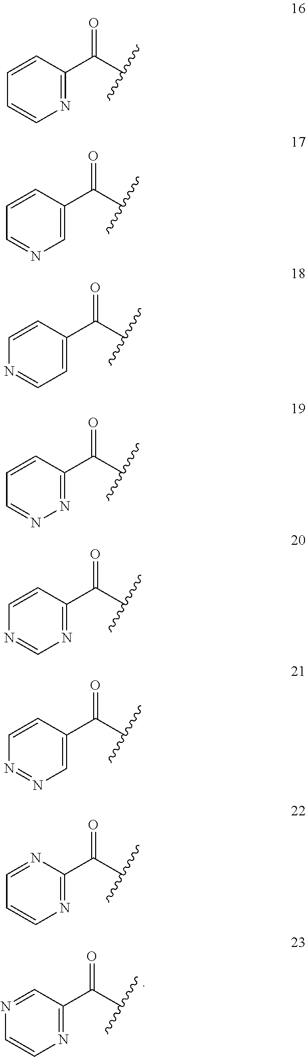

In some embodiments, the composition further comprises a tetracycline antibiotic. In some embodiments, the bacterial infection is caused by a bacterial pathogen selected from Escherichia coli, Acinetobacter baumannii, Pseudomonas aeruginosa, Legionella longbeacha, and Mycobacteria abscessus. In some embodiments, compounds of Formula (I) and Formula (II) have antibacterial activity. In some embodiments, the composition inhibits one or more tetracycline destructase enzymes.

Other objects and features will be in part apparent and in part pointed out hereinafter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings described herein are for illustrative purposes only. The drawings are not intended to limit the scope of the present teachings in any way.

FIG. 1 is a schematic summarizing three aTC-based inhibitor types for TDases: aTC analogs; C9-benzamides/benzylamines; C10-benzoate esters (this work). aTC analogs compete directly with TC for TDase binding to inhibit the coupled path for TC oxidation and directing the TDase down the uncoupled path for release of H2O2. C9-benzamides/benzylamines are bivalent inhibitors that compete with TC and NADPH for TDase binding to block FAD reduction. C10-benzoate esters display mixed inhibition patterns with some analogs inhibiting FAD reduction and other analogs supporting the uncoupled release of H2O2.

FIG. 2A is a graph showing optical absorbance over time (minutes) after treatment with inhibitor 9 against TetX7.

FIG. 2B is a graph showing optical absorbance over time (minutes) after treatment with inhibitor 9 against Tet50.

FIG. 2C is a graph and corresponding image of inhibitor 9 against TetX7. The top shows molecule ion counts over time for the inhibitor [M+H]⁺ and the oxidation product [M+16]⁺ supporting the rapid oxidation of the inhibitor scaffold. The bottom panels show the production of hydrogen peroxide over time using a colorimetric assay.

FIG. 2D is a graph and corresponding image of inhibitor 9 against Tet50. The top shows molecule ion counts over time for the inhibitor [M+H]⁺ and the oxidation product [M+16]⁺ supporting the rapid oxidation of the inhibitor scaffold. The bottom panels show the production of hydrogen peroxide over time using a colorimetric assay.

FIG. 3A is a schematic of the synthesis of C10-Benzoate aTC analog backbone structure for compounds 2-15 and the corresponding R-groups.

FIG. 3B is a schematic of the synthesis of C9-Benzamide aTC analog backbone structure for compounds 16-23 and the corresponding R-groups.

FIG. 4A is a graphical representation of the TetX7 structure bound with FAD and inhibitor 13. Tet(X7) is represented in ribbon and ligands are showed as stick model with carbon in gray (FAD) and yellow (inhibitor 13), oxygen in red, nitrogen in blue, phosphorus in orange.

FIG. 4B is a graphical representation of the inhibitor 13 molecule enclosed in a difference Fo−Fc electron density cage (green) with 2.5 σ level.

FIG. 4C is a graphical representation of the substrate binding site of Tet(X7). Structure of Tet(X7)-inhibitor 13 was superposed on aTC:TetX6 PDB: 8ERO (in dark gray color).

FIG. 5A is a graph of Fractional Inhibitory Concentration Index (FICI) of inhibitor 7 (pink) and inhibitor 8 (blue) against empty vector, Tet50 and TetX7. Graph depicts the plotted MIC values for inhibitor concentration (μg/mL; y-axis) versus tetracycline or tigecycline concentration (μg/mL; x-axis). Triangles represent synergy. Each minimum inhibitory concentration (MIC) value was determined from three independent biological replicates.

FIG. 5B is a set of graphs showing the FICI of aTC (black), inhibitor 7 (pink), and inhibitor 8 (blue) against E. coli (DH5αZ1) and P. aeruginosa (PA 27853, Pa-3). Graph depicts the plotted MIC values for inhibitor concentration (μg/mL; y-axis) versus tetracycline or tigecycline concentration (μg/mL; x-axis). Triangles represent synergy. Each minimum inhibitory concentration (MIC) value was determined from three independent biological replicates.

FIG. 5C is a graph of FICI of inhibitor 7 (pink) and inhibitor 8 (blue) against M. smegmatis (mc2155). Graph depicts the plotted MIC values for inhibitor concentration (μg/mL; y-axis) versus tetracycline or tigecycline concentration (μg/mL; x-axis).

FIG. 5D is a set of graphs showing the FICI of aTC (black), inhibitor 7 (pink), and inhibitor 8 (blue) against E. coli (DH5αZ1) and P. aeruginosa (PA 27853, Pa-3). Graph depicts the plotted MIC values for inhibitor concentration (μg/mL; y-axis) versus tetracycline or tigecycline concentration (μg/mL; x-axis). Triangles represent synergy.

FIG. 6A is a schematic of a mechanistic pathway for O- and N-acylation of C9-amino-aTC. C9-Amides could form via direct N-acylation, or initial kinetic O-acylation followed by a 1,2-O-to-N acyl migration to give the more thermodynamically stable C9-amide product.

FIG. 7A is a set of dose-response plots showing human renal proximal tubule epithelial cell viability for TDase inhibitor aTC, 7, 8, and 9. Data points represent mean % inhibition normalized to the control for two independent trials.

FIG. 7B is a set of dose-response plots showing human renal proximal tubule epithelial cell viability for TDase inhibitor 10, 11, 12, and 13. Data points represent mean % inhibition normalized to the control for two independent trials.

FIG. 7C is a set of dose-response plots showing human renal proximal tubule epithelial cell viability for TDase inhibitor 14, 15, 19, and 20. Data points represent mean % inhibition normalized to the control for two independent trials.

FIG. 8 is a graph showing human red blood cell hemolysis with different compounds.

FIG. 9A is a graph monitoring ester hydrolysis over time (green, 0 h; red, 1 h; blue, 16 h) in E. coli cell lysates containing inhibitor 7.

FIG. 9B is a graph monitoring ester hydrolysis over time (green, 0 h; red, 1 h; blue, 16 h) in E. coli cell lysates containing inhibitor 8.

FIG. 9C is a graph monitoring ester hydrolysis over time (green, 0 h; red, 1 h; blue, 16 h) in E. coli cell lysates containing para-nitrophenyl benzoate via LC-MS.

FIG. 9D is a graph monitoring ester hydrolysis over time (green, 0 h; red, 1 h; blue, 16 h) in E. coli cell lysates containing para-nitrophenyl acetate via A₄₀₅ with variable lysate % to validate esterase activity.

FIG. 10 is a graph showing time-dependent inhibition of TetX7 by compound 11. The graph depicts normalized ion counts corresponding to tetracycline (TC; y-axis) versus time (s; x-axis) in the presence of variable concentrations of compound 11.

FIG. 11A is a set of optical absorbance graphs for compound 3 against TetX7 (top) and Tet50 (bottom) and corresponding plate images showing colorimetric detection of hydrogen peroxide at 0, 5, 30, 90, and 120 minute timepoints.

FIG. 11B is a set of optical absorbance graphs for compound 4 against TetX7 (top) and Tet50 (bottom) and corresponding plate images showing colorimetric detection of hydrogen peroxide at 0, 5, 30, 90, and 120 minute timepoints.

FIG. 11C is a set of optical absorbance graphs for compound 5 against TetX7 (top) and Tet50 (bottom) and corresponding plate images showing colorimetric detection of hydrogen peroxide at 0, 5, 30, 90, and 120 minute timepoints.

FIG. 11D is a set of optical absorbance graphs for compound 6 against TetX7 (top) and Tet50 (bottom) and corresponding plate images showing colorimetric detection of hydrogen peroxide at 0, 5, 30, 90, and 120 minute timepoints.

FIG. 11E is a set of optical absorbance graphs for compound 7 against TetX7 (top) and Tet50 (bottom) and corresponding plate images showing colorimetric detection of hydrogen peroxide at 0, 5, 30, 90, and 120 minute timepoints.

FIG. 11F is a set of optical absorbance graphs for compound 8 against TetX7 (top) and Tet50 (bottom) and corresponding plate images showing colorimetric detection of hydrogen peroxide at 0, 5, 30, 90, and 120 minute timepoints.

FIG. 11G is a set of optical absorbance graphs for compound 10 against TetX7 (top) and Tet50 (bottom) and corresponding plate images showing colorimetric detection of hydrogen peroxide at 0, 5, 30, 90, and 120 minute timepoints.

FIG. 11H is a set of optical absorbance graphs for compound 11 against TetX7 (top) and Tet50 (bottom) and corresponding plate images showing colorimetric detection of hydrogen peroxide at 0, 5, 30, 90, and 120 minute timepoints.

FIG. 11I is a set of optical absorbance graphs for compound 12 against TetX7 (top) and Tet50 (bottom) and corresponding plate images showing colorimetric detection of hydrogen peroxide at 0, 5, 30, 90, and 120 minute timepoints.

FIG. 11J is a set of optical absorbance graphs for compound 13 against TetX7 (top) and Tet50 (bottom) and corresponding plate images showing colorimetric detection of hydrogen peroxide at 0, 5, 30, 90, and 120 minute timepoints.

FIG. 11K is a set of optical absorbance graphs for compound 14 against TetX7 (top) and Tet50 (bottom) and corresponding plate images showing colorimetric detection of hydrogen peroxide at 0, 5, 30, 90, and 120 minute timepoints.

FIG. 12 is a set of graphs showing steady state kinetics of TetX7 (top) and Tet50 (bottom) in the presence of varied compound 7 (left) or 8 (right) with monitoring at 340 nm (absorbance of NADPH). Error bars represent standard deviations for two independent trials. The curve fit indicates that both inhibitors 7 and 8 stimulate NADPH consumption by TetX7 and Tet50. The apparent Kₘ and vₘₐₓ values are as follows: Inhibitor 7, TetX7: Kₘ=26±11 μM, vₘₐₓ=0.03±0.01 min⁻¹; Inhibitor 8, TetX7: Kₘ=110±60 μM, vₘₐₓ=0.054±0.018 min⁻¹; Inhibitor 7, Tet50: Kₘ=37±5 μM, vₘₐₓ=0.19±0.01 min⁻¹; Inhibitor 8, Tet50: Kₘ=67±22 μM, vₘₐₓ=0.13±0.02 min⁻¹.

FIG. 13A is a graphical representation showing the superposition of TetX7-Inhibitor 13 on TetX7 PDB:6WG9 revealed a r.m.s.d. of 0.35 Å over 365 residues. TetX7 is showed in teal ribbon, other TDases are in gray ribbons, ligands are showed as stick models with carbon in gray (FAD) or yellow (inhibitor 13 and aTC), oxygen in red, nitrogen in blue and phosphorous in orange.

FIG. 13B is a graphical representation showing the superposition of TetX7-Inhibitor 13 on TetX6 PDB:8ERO revealed r.m.s.d. of 0.56 Å over 364 residues. TetX7 is showed in teal ribbon, other TDases are in gray ribbons, ligands are showed as stick models with carbon in gray (FAD) or yellow (inhibitor 13 and aTC), oxygen in red, nitrogen in blue and phosphorous in orange.

FIG. 14A is a graphical representation of the structure of inhibitor 3 (top) and a corresponding graph growth rate curves of inhibitor 3 in the presence of 8 μg/mL TC (bottom).

FIG. 14B is a graphical representation of the structure of inhibitor 3=6 (top) and a corresponding graph growth rate curves of inhibitor 3=6 in the presence of 8 μg/mL TC (bottom).

FIG. 15 is a set of images of a western blot showing TetX-HA expression (top) and CarD (bottom) expression in different samples.

FIG. 16 is a set of images showing a growth inhibition assay to determine FICI scores for aTC (top), inhibitor 7 (middle), and inhibitor 8 (bottom) against M. smegmatis mc2155 expressing HA-tagged TetX.

FIG. 17 is a table showing human renal proximal tubule epithelial cell viability when cultured with various inhibitors. The R² cutoff for IC50 curve fit was 0.95. Compounds 10, 12-15, and 19-20 failed this cutoff and was reported as percent inhibition at 100 μM.

FIG. 18 is a table showing the statistics for data collection and refinement of TetX7-inhibitor 13.

FIG. 19 is a table of the bacterial strains used herein.

FIG. 20 is a table of the plasmids used herein.

FIG. 21 is a table of the oligonucleotides used herein (SEQ ID NO:6, SEQ ID NO 7).

### DETAILED DESCRIPTION OF THE DISCLOSURE

The present disclosure is based, at least in part, on the discovery that new C9- and C10-ester derivatives of anhydrotetracycline are able to successfully rescue the activity of tetracycline antibiotics against bacterial pathogens expressing tetracycline resistance enzymes. In exemplary embodiments, these compounds inhibit tetracycline inactivating enzymes (tetracycline destructases) and form the basis of a combination therapy with tetracycline antibiotics to overcome this type of resistance and other types of resistance (e.g. efflux). Accordingly, compounds of the present disclosure exhibit inherent antibacterial activity. As disclosed herein, C10-aTC derivatives exhibit excellent water solubility, strong TC rescue in E. coli, and can be produced with high yield via one-step synthesis

### Chemical Agent

Examples of tetracycline antibiotic rescue agents are described herein, including tetracycline destructase inhibitors. Agents (and compositions comprising one or more agents) can comprise one or more C9-benzamide and C10-benzoate esters of anhydrotetracycline, including compounds of the formula:

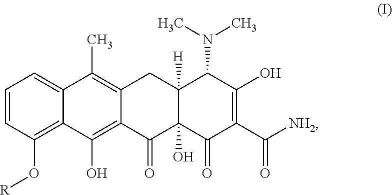

or pharmaceutically acceptable salts thereof. In exemplary embodiments, R is selected from the following:

TABLE 1
R groups for Formula (I)

| Inhibitor # | R group |
|---|---|
| 2 | 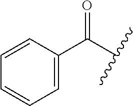 [SMILES: *C(=O)c1ccccc1] |
| 3 | 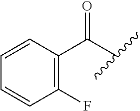 [SMILES: *C(=O)c1ccccc1F] |
| 4 | 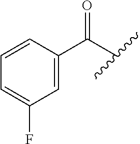 [SMILES: *C(=O)c1cccc(F)c1] |
| 5 | 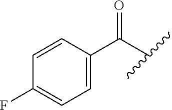 [SMILES: *C(=O)c1ccc(F)cc1] |
| 6 | 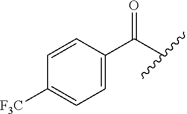 [SMILES: *C(=O)c1ccc(C(F)(F)F)cc1] |
| 7 | 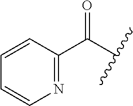 [SMILES: *C(=O)c1ccccn1] |
| 8 | 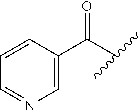 [SMILES: *C(=O)c1cccnc1] |
| 9 | 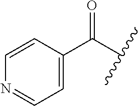 [SMILES: *C(=O)c1ccncc1] |
| 10 | 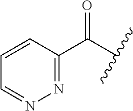 [SMILES: *C(=O)c1cccnn1] |
| 11 | 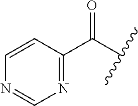 [SMILES: *C(=O)c1ccncn1] |
| 12 | 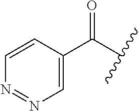 [SMILES: *C(=O)c1ccnnc1] |
| 13 | 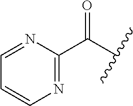 [SMILES: *C(=O)c1ncccn1] |
| 14 | 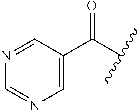 [SMILES: *C(=O)c1cncnc1] |
| 15 | 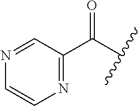 [SMILES: *C(=O)c1cnccn1] |

Example of other agents (and compositions comprising one or more agents) can comprise one or more C9-benzamide of anhydrotetracycline, including compounds of the formula:

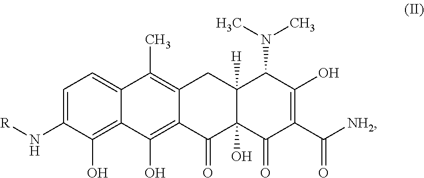

or pharmaceutically acceptable salts thereof. In exemplary embodiments, R is selected from the following:

TABLE 2
R groups for Formula (II)

| Inhibitor # | R group |
|---|---|
| 16 | 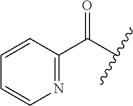 [SMILES: *C(=O)c1ccccn1] |
| 17 | 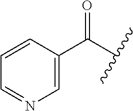 [SMILES: *C(=O)c1cccnc1] |
| 18 | 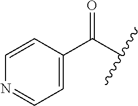 [SMILES: *C(=O)c1ccncc1] |
| 19 | 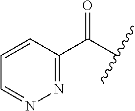 [SMILES: *C(=O)c1cccnn1] |
| 20 | 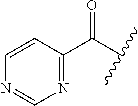 [SMILES: *C(=O)c1ccncn1] |
| 21 | 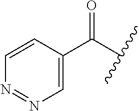 [SMILES: *C(=O)c1ccnnc1] |
| 22 | 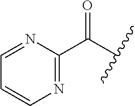 [SMILES: *C(=O)c1ncccn1] |
| 23 | 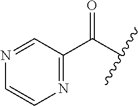 [SMILES: *C(=O)c1cnccn1] |

Further, the formulas, analogs, and R groups can be optionally substituted or functionalized with one or more groups independently selected from the group consisting of hydroxyl; C₁₋₁₀alkyl hydroxyl; amine; C₁₋₁₀-carboxylic acid; C₁₋₁₀-carboxyl; straight chain or branched C₁₋₁₀-alkyl, optionally containing unsaturation; a C₂₋₁₀cycloalkyl optionally containing unsaturation or one oxygen or nitrogen atom; straight chain or branched C₁₋₁₀-alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀-alkyl hydroxyl; amine; C₁₋₁₀-carboxyl; C₁₋₁₀carboxylic acid; C₁₋₁₀-carboxyl; straight chain or branched C₁₋₁₀-alkyl, optionally containing unsaturation; straight chain or branched C₁₋₁₀-alkyl amine, optionally containing unsaturation; a C₂₋₁₀cycloalkyl optionally containing unsaturation or one oxygen or nitrogen atom; straight chain or branched C₁₋₁₀-alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀-alkyl hydroxyl; amine; C₁₋₁₀carboxylic acid; C₁₋₁₀-carboxyl; straight chain or branched C₁₋₁₀-alkyl, optionally containing unsaturation; straight chain or branched C₁₋₁₀-alkyl amine, optionally containing unsaturation; a C₂₋₁₀cycloalkyl optionally containing unsaturation or one oxygen or nitrogen atom; heterocyclyl; straight chain or branched C₁₋₁₀-alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “carbonyl”, as used herein, unless otherwise indicated, can include a functional group consisting of a carbon atom double-bonded to an oxygen atom (C═0). The “carbonyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O-cyclooctyl, —O— cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH2-cyclooctyl, —O—CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein. Examples of -lower alkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S, and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “heterocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example, water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or another counterion. The counterion may be any organic or inorganic moiety that stabilizes the charge on the parent compound. Furthermore, a pharmaceutically acceptable salt may have more than one charged atom in its structure. In instances where multiple charged atoms are part of the pharmaceutically acceptable salt, the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc., may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating or reversing bacterial infection in a subject in need thereof via administration of a therapeutically effective amount of a rescue agent or compositions thereof, so as to rescue antibiotic activity and effectiveness. Methods for preventing bacterial infection via administration of a rescue agent (or compositions thereof) described herein are also contemplated.

Methods described herein are generally performed on a subject in need thereof. A subject in need of the therapeutic methods described herein can be a subject having, diagnosed with, or suspected of having a bacterial infection. In some embodiments a subject may be at risk for bacterial infection, such that the agents and compositions are administered for prophylactic purposes. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a rescue agent (or compositions thereof) is, for example, an amount that would cause the desired therapeutic effect in a subject while minimizing undesired side effects. In various embodiments, an effective amount of a rescue agent (or compositions thereof) described herein can substantially inhibit, slow the progress of, or limit the development of bacterial infection.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a rescue agent or compositions thereof can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to rescue antibiotic activity and effectiveness.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes reversing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of a rescue agent or compositions thereof can occur as a single event or over a time course of treatment. For example, a rescue agent or compositions thereof can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for bacterial infection, particularly advanced and/or antibiotic resistant infections.

A rescue agent or compositions thereof can be administered simultaneously or sequentially with another agent, such as an antibiotic, an anti-inflammatory, or another agent. For example, a rescue agent or compositions thereof can be administered simultaneously with another agent, such as an antibiotic or an anti-inflammatory. Simultaneous administration can occur through administration of separate compositions, each containing one or more of a rescue agent or compositions thereof, an antibiotic, an anti-inflammatory, or another agent. Simultaneous administration can occur through administration of one composition containing two or more of a rescue agent or compositions thereof, an antibiotic, an anti-inflammatory, or another agent. A rescue agent or compositions thereof can be administered sequentially with an antibiotic, an anti-inflammatory, or another agent. For example, a rescue agent or compositions thereof can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22(3):659-661, 2008, which is incorporated herein by reference):

$$HED ( mg / kg ) = Animal dose ( mg / kg ) \times ( Animal {K}_{m} / Human {K}_{m} )$$

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the rescue agent or compositions thereof may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, a rescue agent or compositions thereof, including at least one compound of Formula (I), may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Cell Therapy

Cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting T-cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation has gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogeneic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Screening

Also provided are screening methods.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to the rescue agents, destructase inhibitors, and/or compounds of formula (I) as disclosed herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—C10-Benzoate Esters of Anhydrotetracycline Inhibit Tetracycline Destructases and Recover Tetracycline Antibacterial Activity

In this example, anhydrotetracycline (aTC) derivatives were designed and evaluated as therapies to overcome TDase-mediated antibiotic resistance.

Tetracyclines (TCs) are an important class of antibiotics threatened by enzymatic inactivation. These tetracycline-inactivating enzymes, also known as tetracycline destructases (TDases), are a subfamily of class A flavin monooxygenases (FMOs) that catalyze hydroxyl group transfer and oxygen insertion (Baeyer-Villiger type) reactions on TC substrate scaffolds. Semisynthetic modification of TCs (e.g., tigecycline, omadacycline, eravacycline, and sarecycline) has proven effective in evading certain resistance mechanisms, such as ribosomal protection and efflux, but does not protect against TDase-mediated resistance. Shown herein is the design, synthesis, and evaluation of a new series of 22 semisynthetic TDase inhibitors that explore D-ring substitution of anhydrotetracycline (aTC) including 14 C10-benzoate ester and eight C9-benzamides. Overall, the C10-benzoate esters displayed enhanced bioactivity and water solubility compared to the corresponding C9-benzamides featuring the same heterocyclic aryl side chains. The C10-benzoate ester derivatives of aTC were prepared in a high-yield one-step synthesis without the need for protecting groups. The C10-esters are water-soluble, stable toward hydrolysis, and display dose-dependent rescue of tetracycline antibiotic activity in E. coli expressing two types of tetracycline destructases, represented by TetX7 (Type 1) and Tet50 (Type 2). The best inhibitors recovered tetracycline antibiotic activity at concentrations as low as 2 μM, producing synergistic scores <0.5 in the fractional inhibitory concentration index (FICI) against TDase-expressing strains of E. coli and clinical P. aeruginosa. The C10-benzoate ester derivatives of aTC reported here are promising new leads for the development of tetracycline drug combination therapies to overcome TDase-mediated antibiotic resistance.

### Introduction

Tetracyclines (TCs) are essential antibacterial agents used for treating a wide range of clinical infections caused by aerobic, anaerobic, Gram-positive, and Gram-negative pathogens. Third generation TCs including tigecycline (Tig), eravacycline, and omadacycline are considered drugs of last resort for treating multidrug resistant (MDR) pathogens such as MDR Acinetobacter baumannii. These third-generation TCs overcome traditional clinical resistance mechanisms including efflux pumps and ribosome protection proteins, but a new resistance mechanism is emerging in the form of tetracycline-inactivating enzymes known as tetracycline destructases (TDases). TDases have been found within inducible antibiotic resistance operons in MDR bacterial pathogens including A. baumannii, Pseudomonas aeruginosa, Legionella longbeachae, and Mycobacterium abscessus. TDases can inactivate all generations of the TC antibiotic family and threaten the clinical viability of tetracycline drugs of last resort.

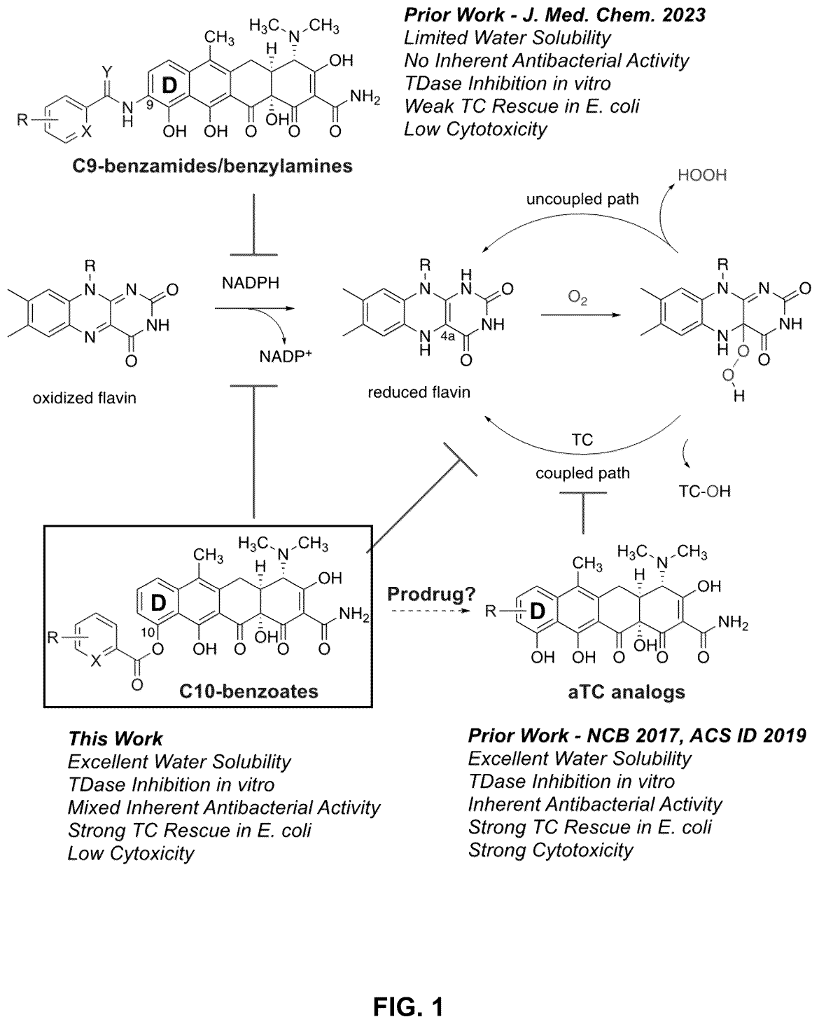

The dissemination of TDase genes in the environment has been characterized, including hospital settings. The occurrence of TDases is increasing in clinical pathogens and there is soon to be a serious clinical need for TDase inhibitors, much like the standard of care established for beta-lactam antibiotic and beta-lactamase inhibitor combination therapies. Anhydrotetracycline (aTC) and its analogs are potent broad-spectrum inhibitors of the two major classes of TDases (Type 1 and 2) found in bacterial pathogens (FIG. 1). However, aTC is cytotoxic and certain Type 1 TDases are capable of oxidizing aTC, which led us to develop a series of bivalent TDase inhibitors based on the fusion of aTC and benzamide linked biomimetics of nicotinamide placed at the C9-position of the D-ring. These bivalent inhibitors are competitive with both TC and NADPH substrates to block the initial reduction of FAD and prevent turnover of both the inhibitor and the substrate. This strategy results in more efficient TDase inhibition and preserves the inhibitor scaffold by blocking inhibitor oxidation. Two types of the inhibitors comprise linkages at the C9-position of aTC: benzamides and benzylamines. The benzamides are more potent inhibitors but have limited water solubility and poor cell permeability consistent with the observed weak rescue effect in E. coli when dosed in combination with TC antibiotics. Shown herein are C10-ester TDase inhibitors which are fully water-soluble at all concentrations tested, and have improved whole cell activity compared to C9-amides for the rescue of growth inhibition by TC and Tig against E. coli, P. aeruginosa, and Mycobacterium smegmatis expressing Type 1 (TetX or TetX7) and Type 2 (Tet50) TDases.

### Results

### Design and Synthesis of C10-Benzoate Inhibitors

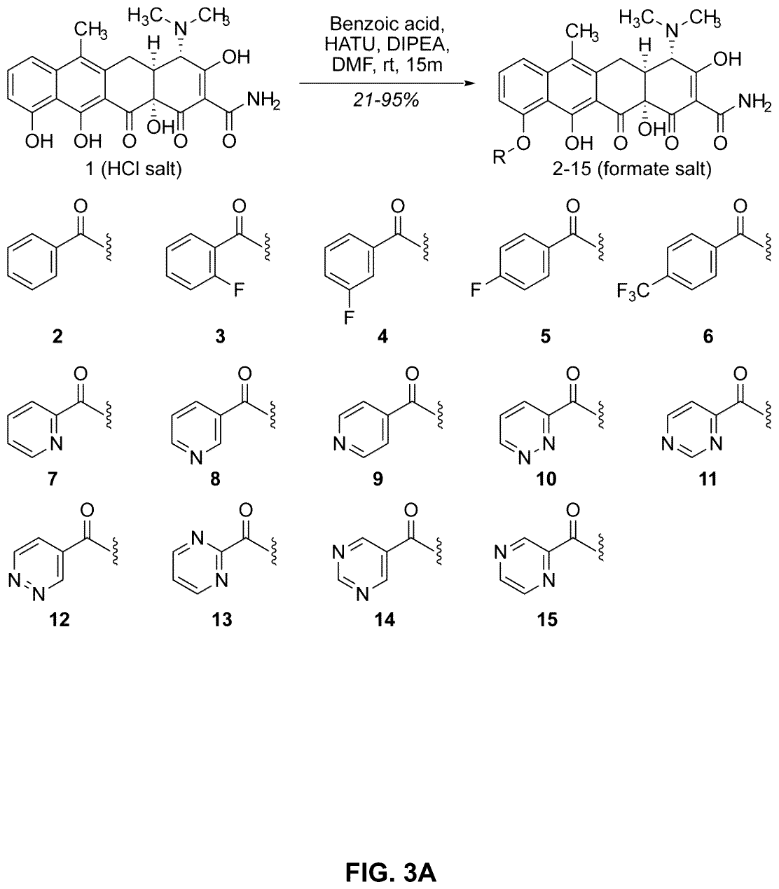

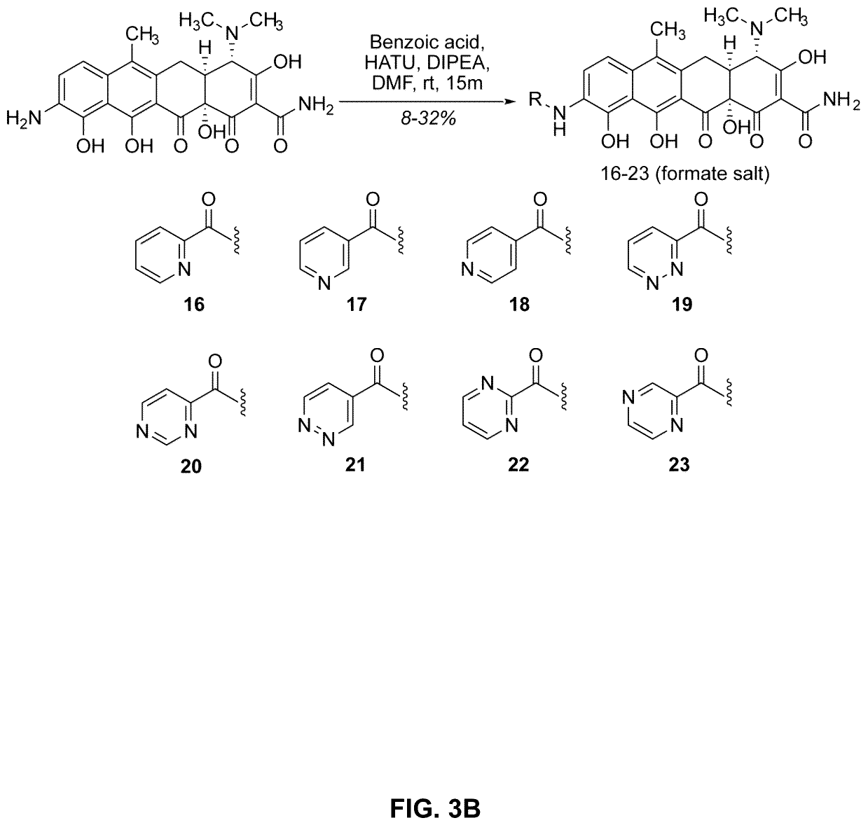

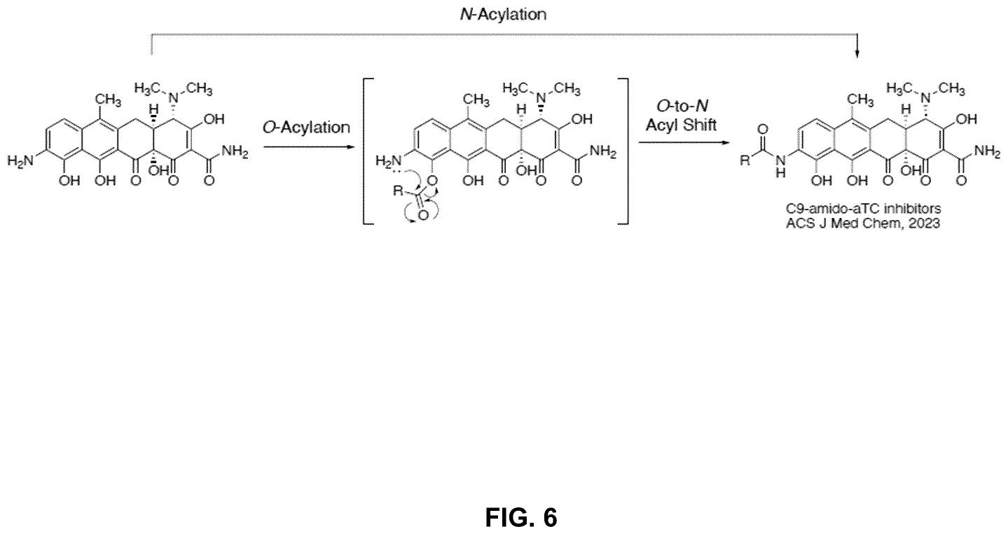

To improve the water solubility and cell permeability of aTC-based inhibitors, the aTC scaffold was modified at different positions. While synthesizing C9-amides from C9-amino aTC, there was the formation of an additional product that was hypothesized to be the C10-ester which spontaneously rearranges to make the more thermodynamically stable C9-amide product (FIG. 6). To test this hypothesis, aTC was treated with benzoic acids in the presence of a coupling reagent (HATU) under basic conditions (DIPEA) in DMF solvent. A rapid (<15 min), clean, and near complete conversion of aTC (1) to the corresponding C10-esters 2-15 was seen (FIG. 3A). It was confirmed that the chemoselective esterification of the C10-hydroxyl group through complete structural elucidation via multidimensional NMR analysis including COSY, TOCSY, HMBC, and HSQC. There was a consistent downfield shift of the C9 hydrogen in the ¹H NMR spectrum of the C10 esters relative to aTC due to the exposure of the C9-H to the deshielding effects of the C10 ester carbonyl. In aTC, the chemical shift order of the D-ring hydrogens appears as C8 (t, 7.6 ppm)>C9 (d, 7.5 ppm)>C7 (d, 6.9 ppm). In C10 ester 2, for example, the chemical shift order of the D-ring hydrogens appears as C9 (d, 8.1 ppm)>C8 (t, 7.8 ppm)>C7 (d, 7.4 ppm). There was a similar pattern for all the C10 ester derivatives. The yields after purification by prep-HPLC ranged from 21 to 95%. This allowed for the rapid synthesis of 14 analogs derived from substituted benzoic acid and heterocyclic aromatic acids to explore the potential of C10 esters as inhibitors of TDases. For direct comparison, an additional 8 aTC analogs was synthesized as C9-amides 16-23 using the same aromatic acids as C10-esters 7-13 and 15 (FIG. 3B). While amide coupling with C9-NH2-aTC is also a one-step synthesis, the yields for this reaction compared to C10-esterification are significantly lower ranging from 8 to 32%. Further, the preparation of C9-NH2-aTC requires two additional synthetic steps starting from the commercially available aTC. The C10-ester derivatives are promising lead compounds for the development of effective TDase inhibitors as potential combination therapies with TC antibiotics.

### Compound Stability and Cytotoxicity

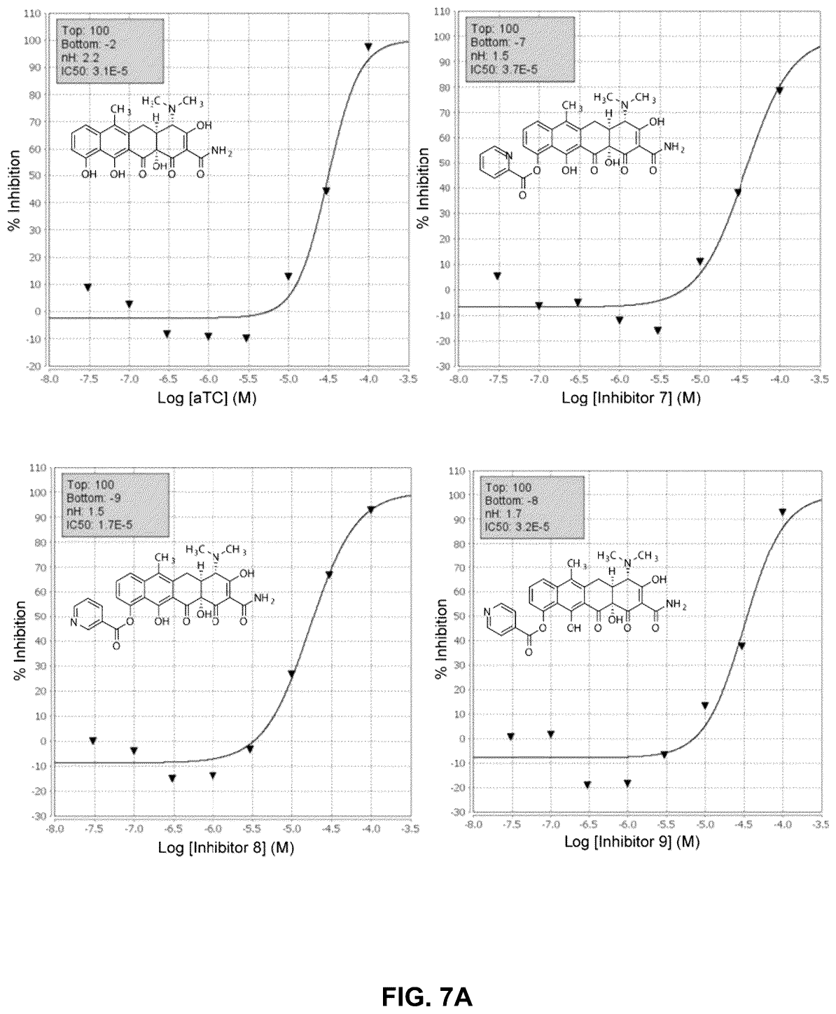

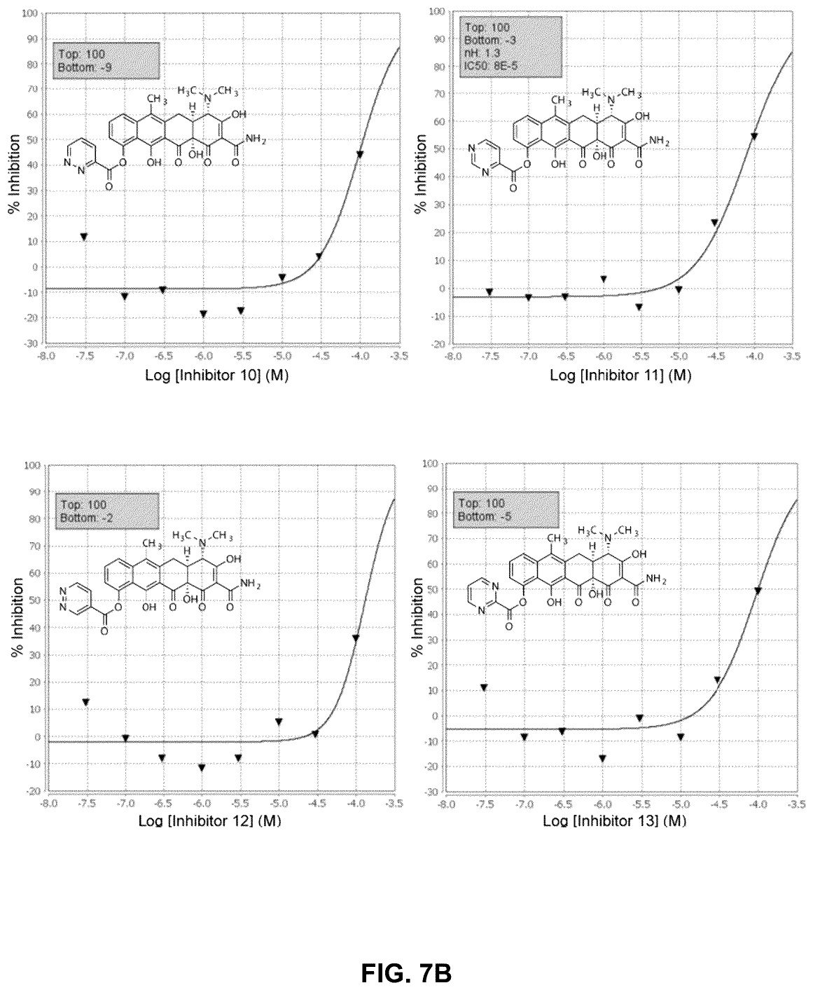

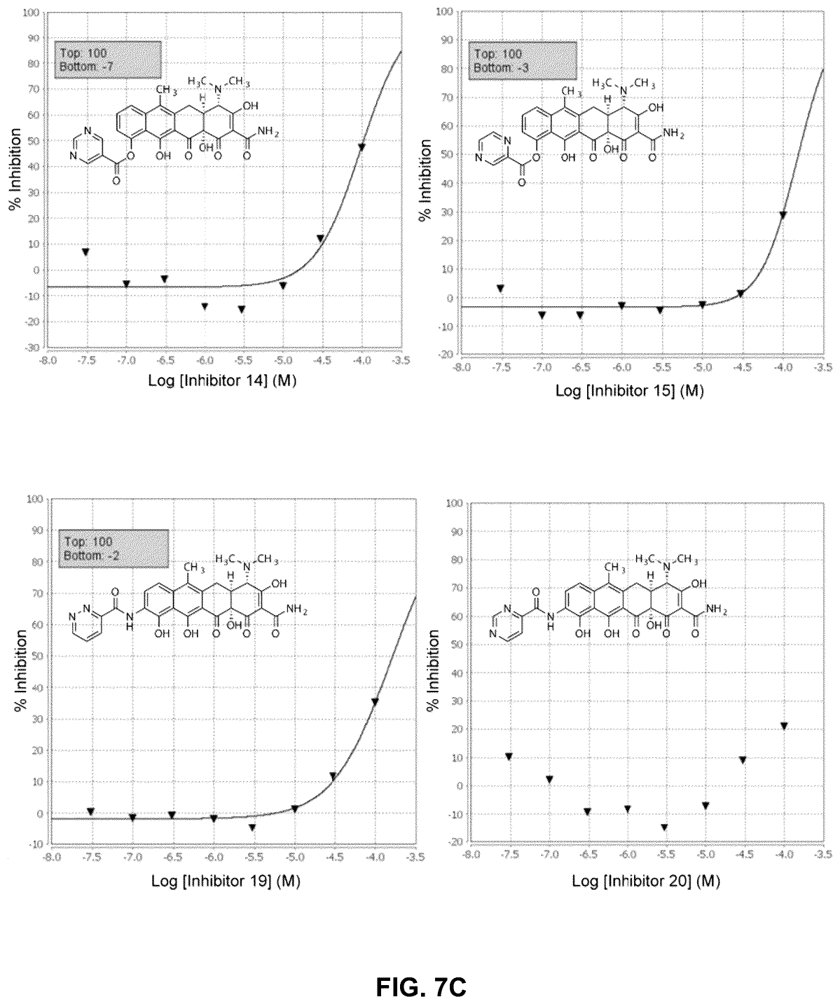

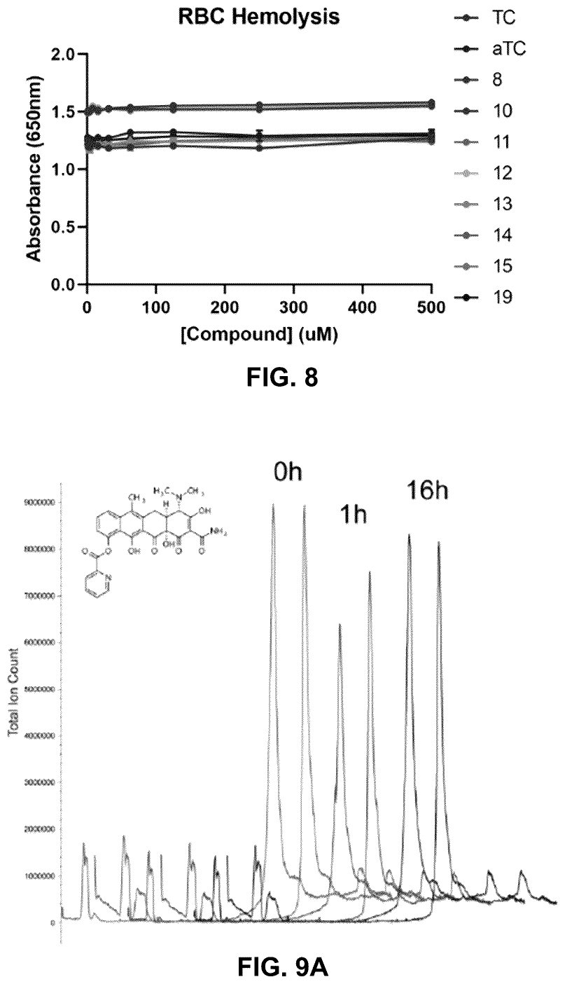

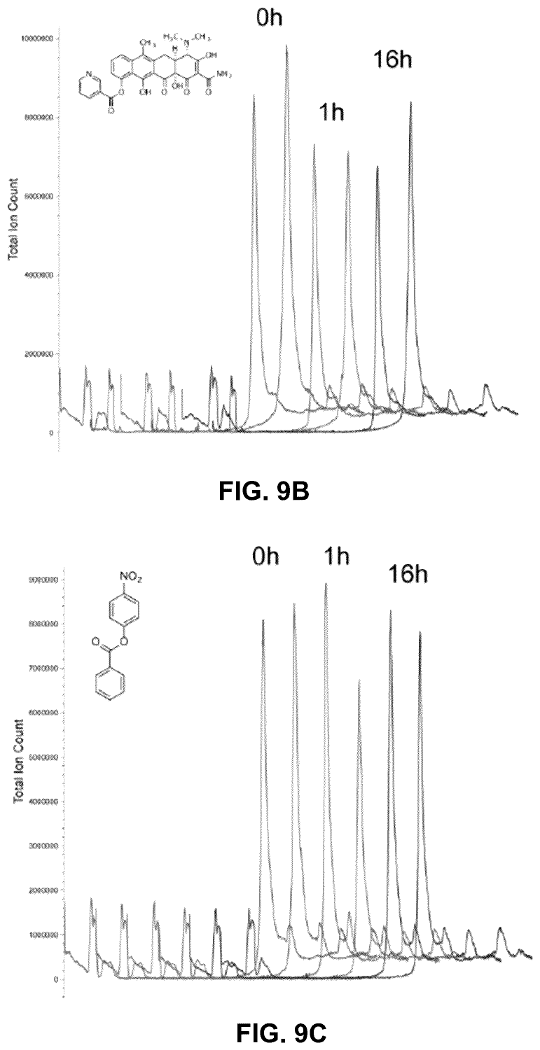

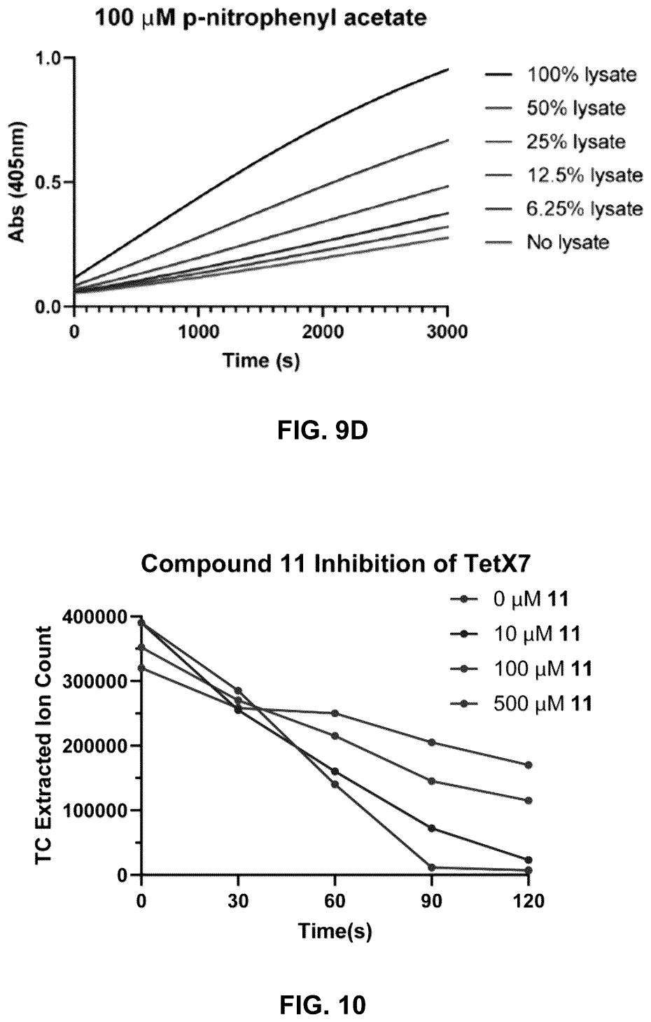

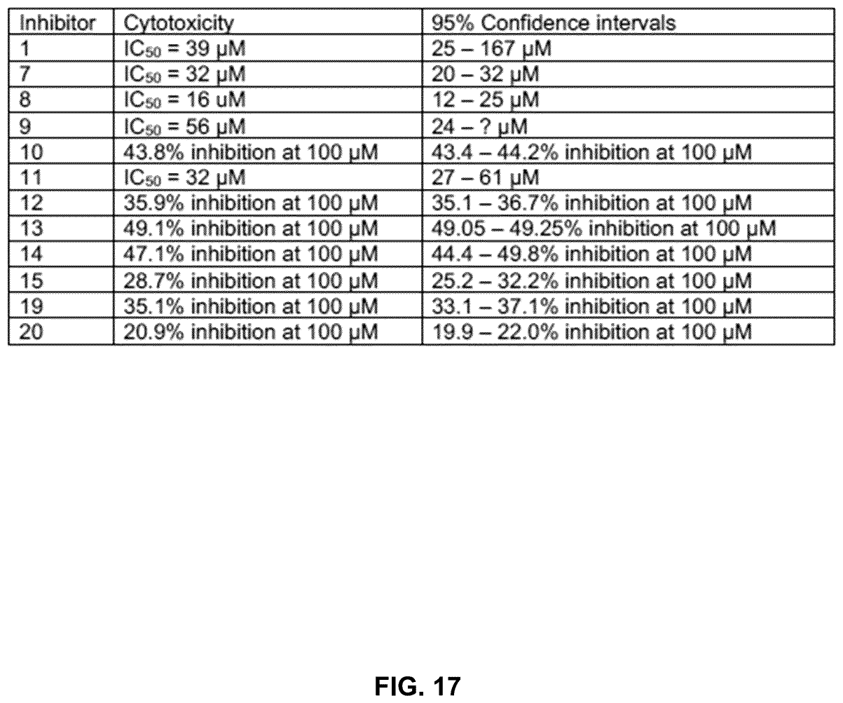

To compare the toxicity of the C10-ester inhibitors to aTC which is known to display cytotoxicity, an assays for cell viability was performed using human renal proximal tubule epithelial cells (contracted through Eurofins, St. Louis, MO). Relative to the positive control staurosporine (IC₅₀˜0.1-0.3 μg/mL), none of the compounds showed significant toxicity below 30 μM (15 μg/mL which provides a viable therapeutic window based on the observed TC rescue at 2 μg/mL inhibitor (FIG. 7A, FIG. 7B, FIG. 7C, FIG. 17). Also TC, aTC, and compounds 8, 10-15, and 19 were tested for hemolysis in human red blood cells. No hemolysis was observed at concentrations up to 500 μM (˜250 μg/mL) (FIG. 8). There was the possibility that the C10-esters are prodrugs that release potentially toxic aTC upon hydrolysis, either esterase-catalyzed or nonenzymatic hydrolysis. To test this, a hydrolytic stability studies on compounds 7 and 8 in bacterial cell lysates (E. coli) was performed. The positive control, p-NO₂-phenyl acetate, caused a time- and lysate-dependent increase in A₄₀₅ as expected. Compounds 7 and 8 were stable in the cell lysate according to the LC-MS analysis and the appearance of free aTC was not observed (FIG. 9A, FIG. 9B, FIG. 9C, FIG. 9D). A p-NO₂-phenyl benzoate control compound was also used to model the stability of a general benzoate ester. Like benzoate esters 7 and 8, the p-NO₂-phenyl benzoate control was stable in the cell lysate, suggesting that unlike acetate esters the benzoate esters are not vulnerable to hydrolysis by bacterial esterases.

C10-Benzoate Inhibitors Bind TDases with Mixed Binding Modes

Explored here is the ability of the synthetic C10-esters and C9-amides to inhibit TetX7 and Tet50 in vitro using a standard NADPH/TC consumption assay. These experiments gave mixed results indicating that some of the compounds appear to be competitive inhibitors while others promote the consumption of NADPH making it difficult to extrapolate a reliable IC50 value by monitoring for NADPH or TC consumption over time. It was observed that ligands including aTC and TC can bind to Type 1 and 2 TDases via multiple binding modes. This sampling of binding modes may confound the in vitro optical absorbance assays resulting in a mixed interpretation of inhibition vs enzymatic turnover (consumption of NADPH and/or TC). Alternatively, overlapping absorbance spectra for the substrate, inhibitor, and oxidation products of the substrate and inhibitor could create interfering signals. To avoid these issues with optical absorbance-based assays and to determine whether the C10-esters protect TCs from TDase-catalyzed degradation, an in vitro TDase (TetX7) reaction was monitored by LC-MS to quantify the concentration of TC over time in the presence or absence of C10-ester 11 as a representative inhibitor (FIG. 10). It was observed that increasing concentrations of C10-ester 11 reduced the rate of TC oxidation over time suggesting that C10-ester 11 is a dose dependent inhibitor of TetX7.

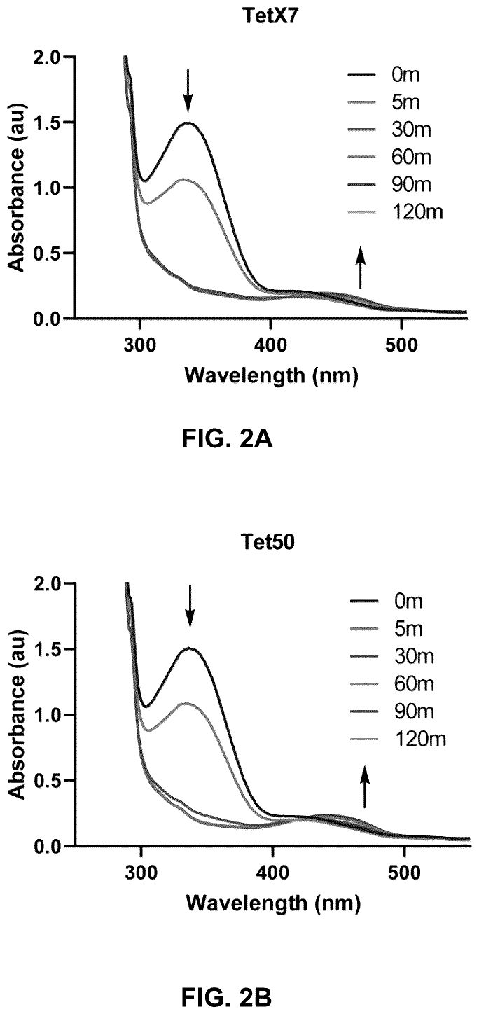

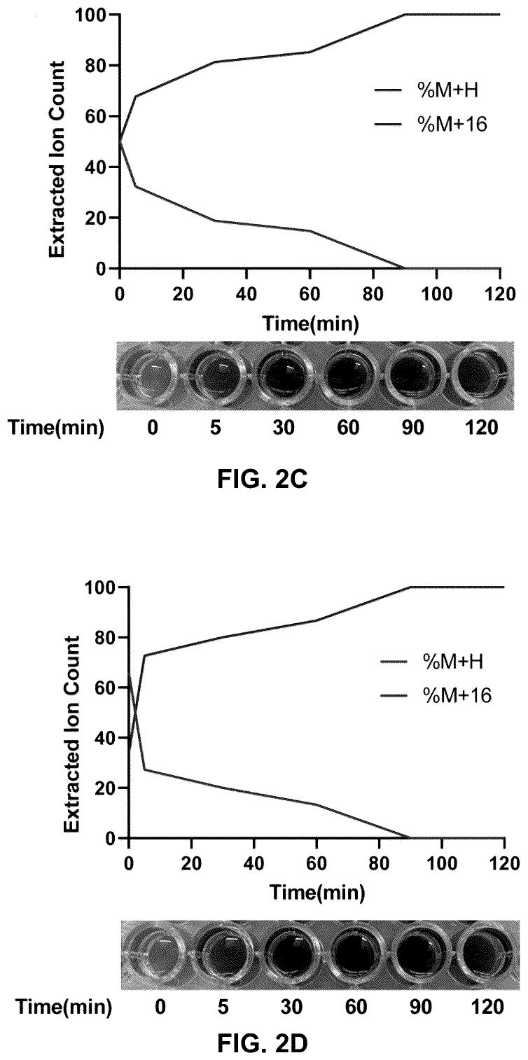

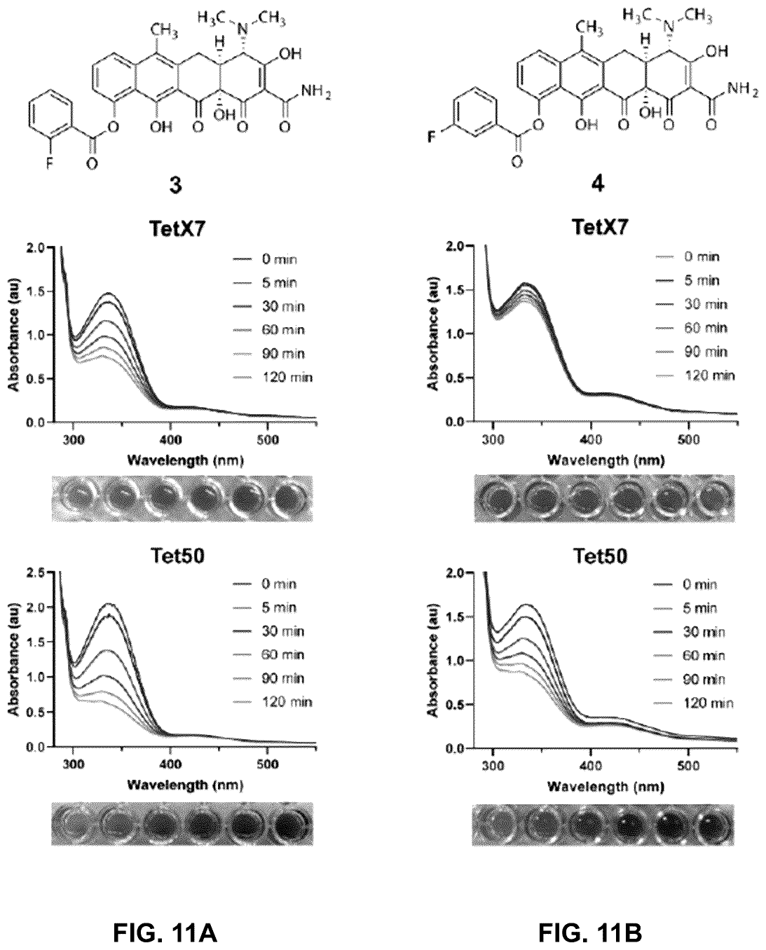

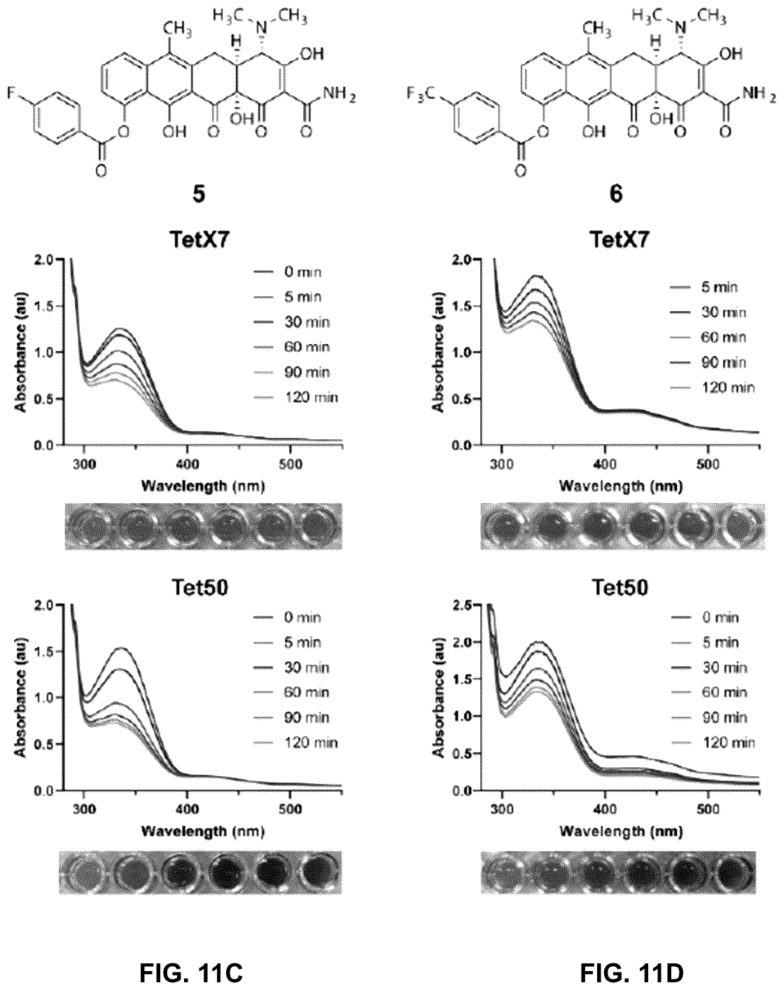

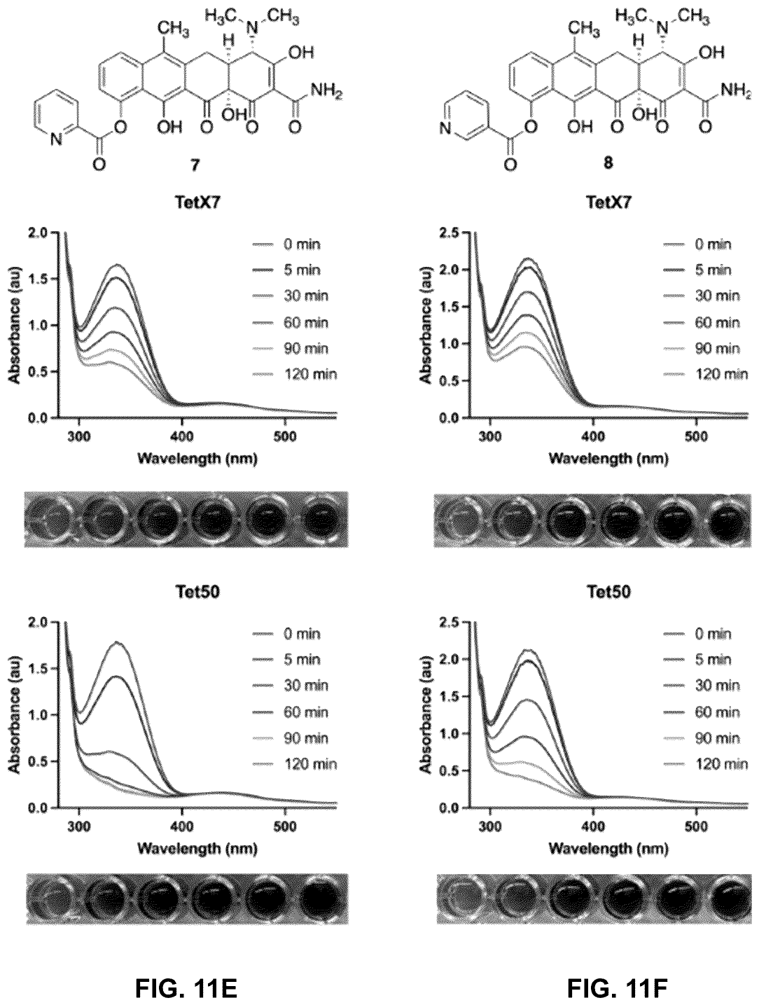

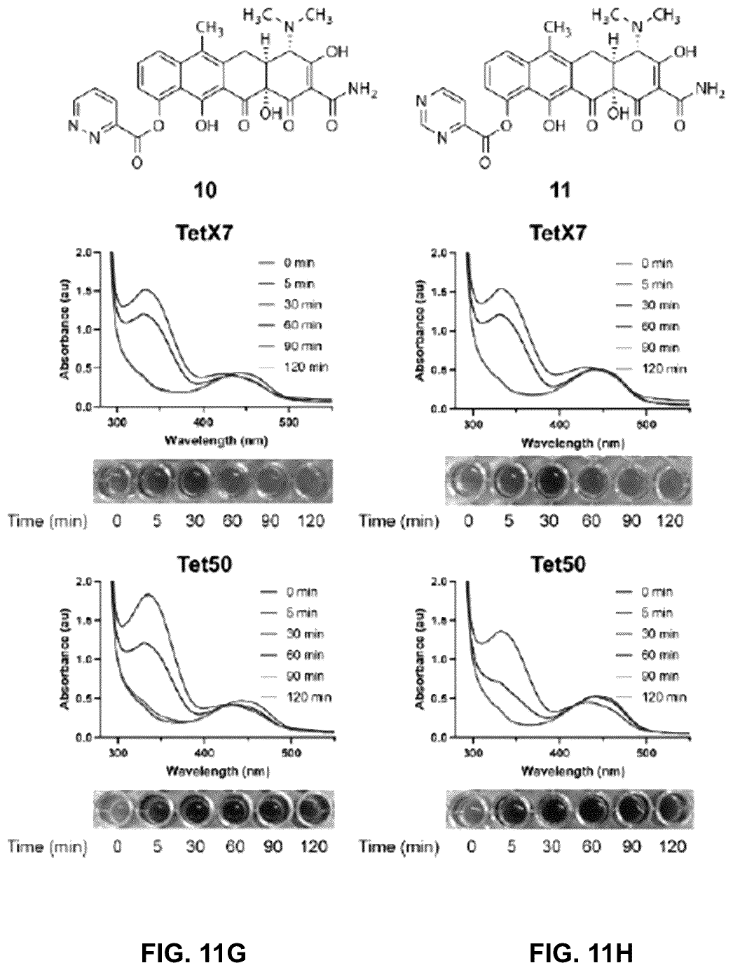

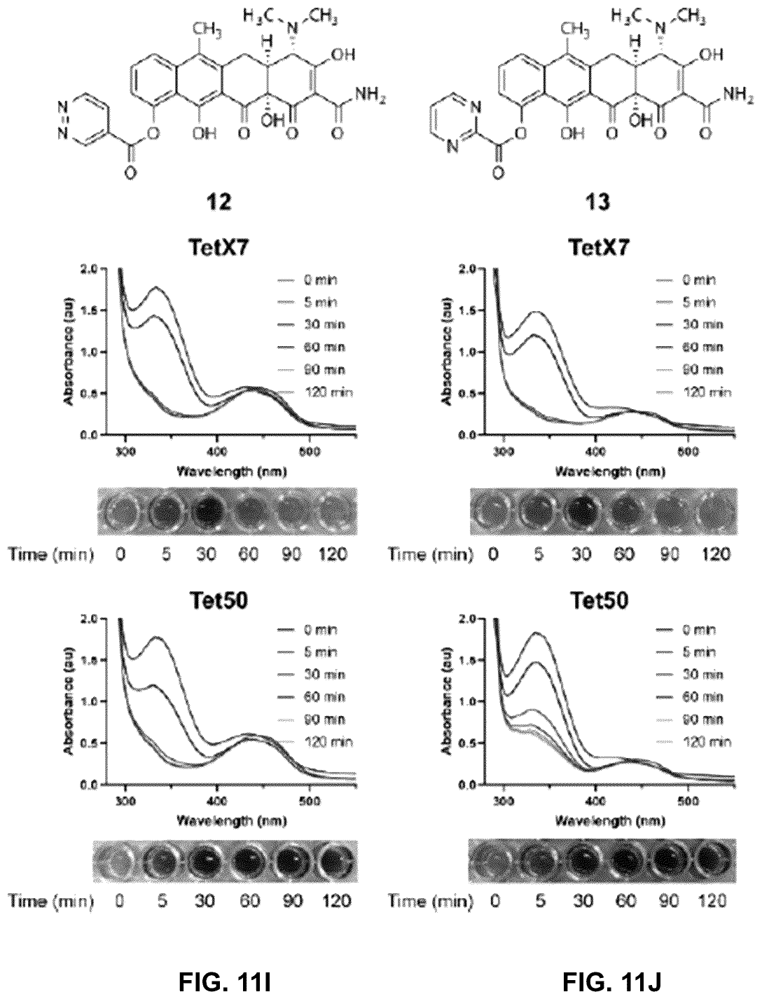

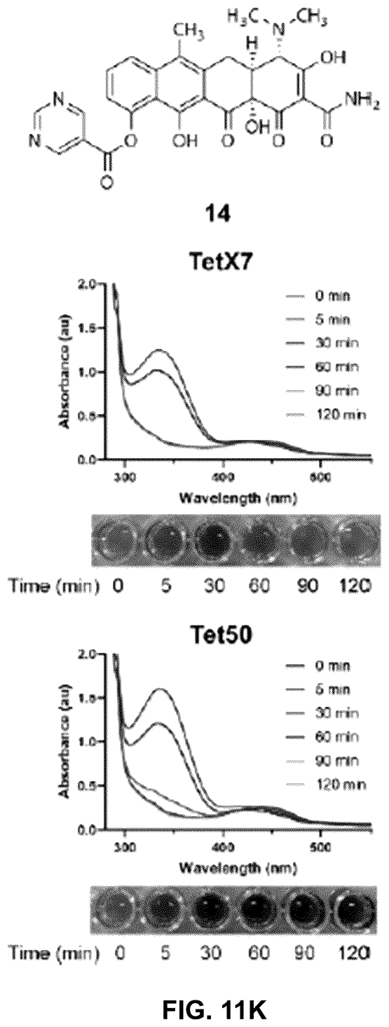

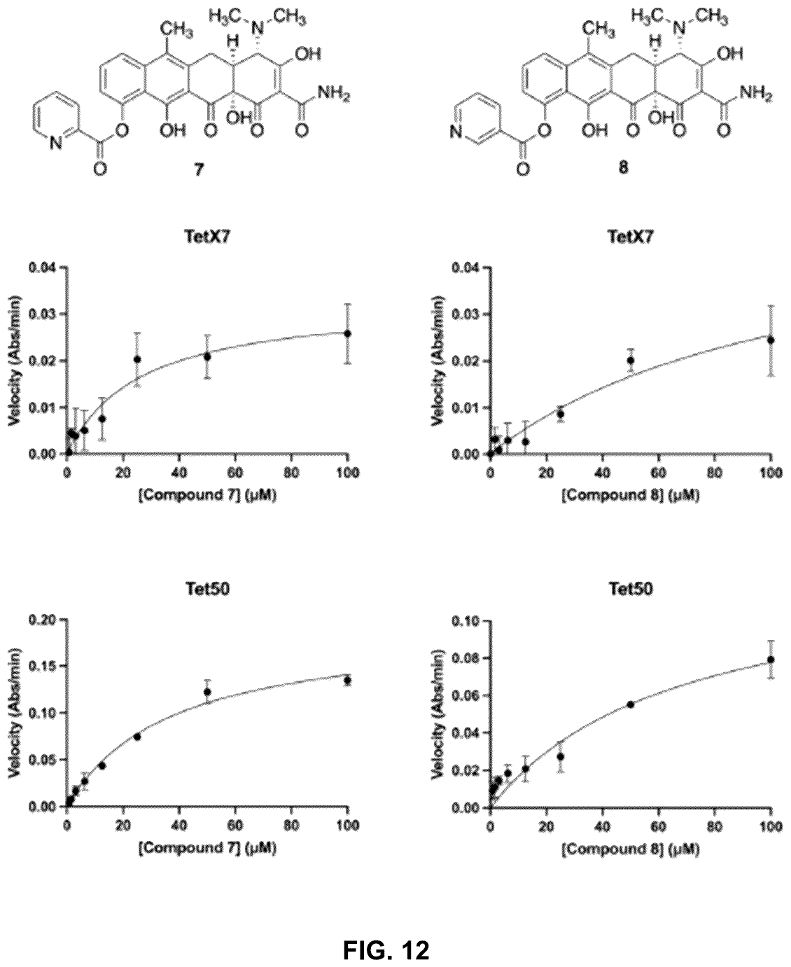

Here, the mechanism of TDase inhibition by C10-esters 3-14 was investigated using optical absorbance to monitor the consumption of NADPH (λmax=340 nm) and the inhibitor (λmax=450 nm). Also tested was the reactions for peroxide formation to determine whether the FAD is reduced by NADPH, leading to the formation of a peroxyflavin intermediate that decomposes to release hydrogen peroxide in an uncoupled reaction (FIG. 2A, FIG. 2B, FIG. 2C, FIG. 2D, FIG. 11A, FIG. 11B, FIG. 11C, FIG. 11D, FIG. 11E, FIG. 11F, FIG. 11G, FIG. 11H, FIG. 111, FIG. 11J, FIG. 11K). These in vitro assays support that the heterocycle-containing inhibitors 7-14 stimulate time-dependent NADPH consumption and hydrogen peroxide production, while the substituted benzene derivatives 3-6 appear to inhibit NADPH consumption similarly to the previously reported C9-aTC derivatives. It was recently reported that aTC increases the rate of NADPH consumption in a dose dependent manner for Tet(X7) (Kₐₚₚ=2.1±0.3 μM, Vₘₐₓ=0.043±0.002 min⁻¹) and Tet(50) (Kₐₚₚ=2.0±0.3 μM, Vₘₐₓ=0.24±0.01 min⁻¹). Similarly, the apparent steady-state kinetics of NADPH consumption for TetX7 and Tet50 was analyzed in the presence of increasing concentrations inhibitors 7 and 8 by monitoring optical absorbance at 340 nm. These results show a similar dose-dependent increase in NADPH consumption for TetX7+7 (Kₐₚₚ=26±11 μM, Vₘₐₓ=0.03±0.01 min⁻¹), Tet50+7 (Kₐₚₚ=37±5 μM, Vₘₐₓ=0.19±0.01 min⁻¹), TetX7+8 (Kₐₚₚ=110±60 μM, Vₘₐₓ=0.05±0.02 min⁻¹), and Tet50+8 (Kₐₚₚ=67±22 μM, Vₘₐₓ=0.13±0.02 min⁻¹) (FIG. 12). However, the A₄₅₀ did not change significantly for any of the inhibitors tested, indicating that the core aTC scaffold of the C10-esters is not being oxidized by TetX7 or Tet50. Additionally, there was a clear formation of a [M+O+H]⁺ oxidation product derived from inhibitor 9 which was detected via LC-MS analysis of the reaction mixture. This could be due to oxidation of the C10-ester aryl side chain which preserves the conjugated chromophore of the aTC derivatives responsible for the absorbance at 450 nm (FIG. 2A, FIG. 2B, FIG. 2C, FIG. 2D). Taken together, these results support that the C10-benzoate ester derivatives of aTC bind TDases with low μM affinity and inhibit TDase activity through a competitive binding mode for the TC and/or NADPH binding sites depending on the identity of the aryl ester side chain. The C10-esters could act as competitive inhibitors where oxidation of the aryl side chain protects the aTC core and allows the oxidation product to retain binding affinity for TDases. Similarly, aTC has been shown to undergo slow oxidation, presumably at C11a, by Type I TDases including TetX7, yet aTC is still effective at rescuing TC activity in TDase-expressing cells either because the aTC oxidation product retains inhibition or because of favorable differences in the Kₘ/k_{cat} of aTC compared to TC toward TDases. The mixed results among the C10-ester inhibitors related to stimulating or slowing NADPH consumption might be caused by a sampling of different inhibitor binding modes, which has been shown can influence the positioning of the dynamic FAD cofactor either into the active site (active state) or exposed to solvent outside of the active site (inactive state).

### Cocrystal Structure Shows C10-Ester Inhibitor in the Substrate Binding Mode

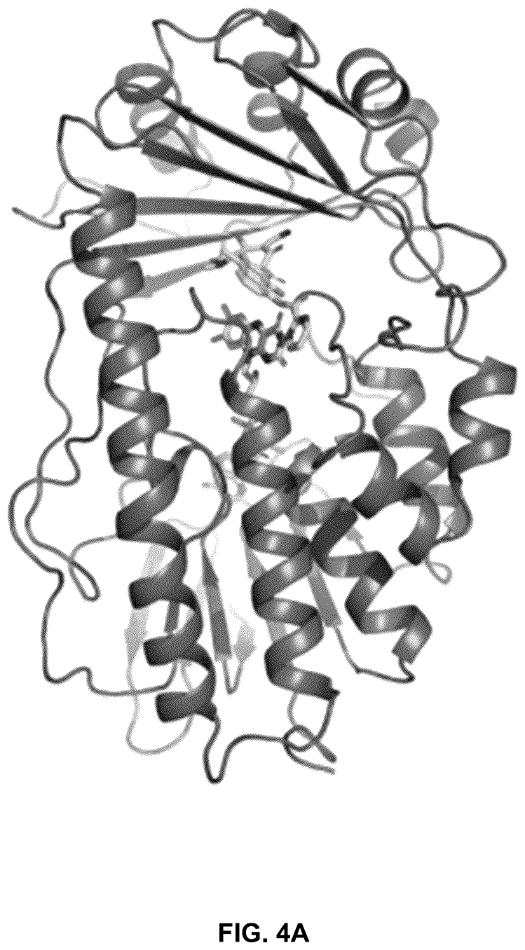

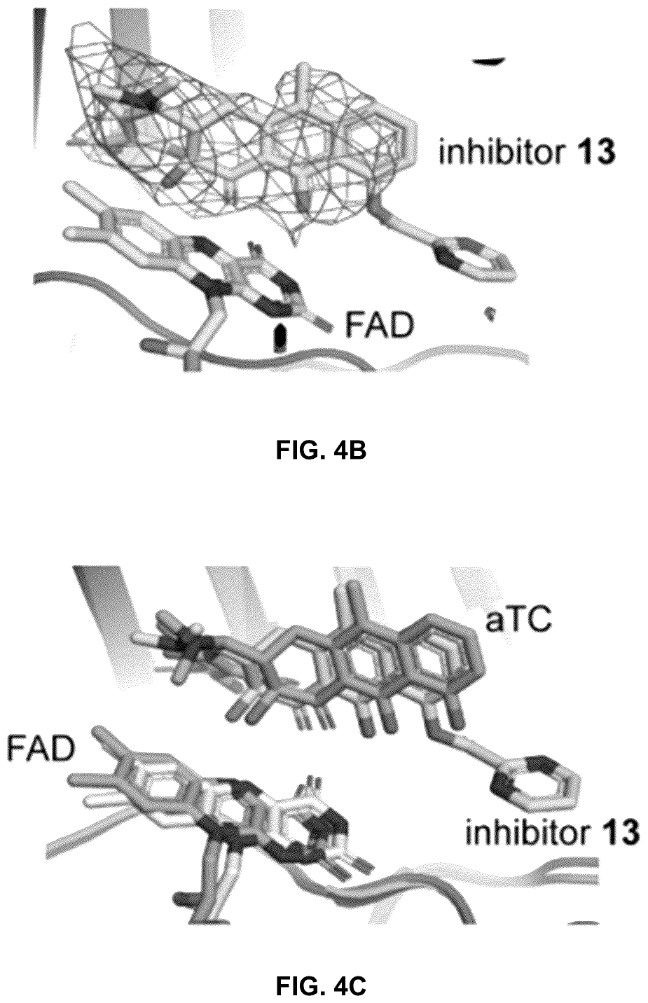

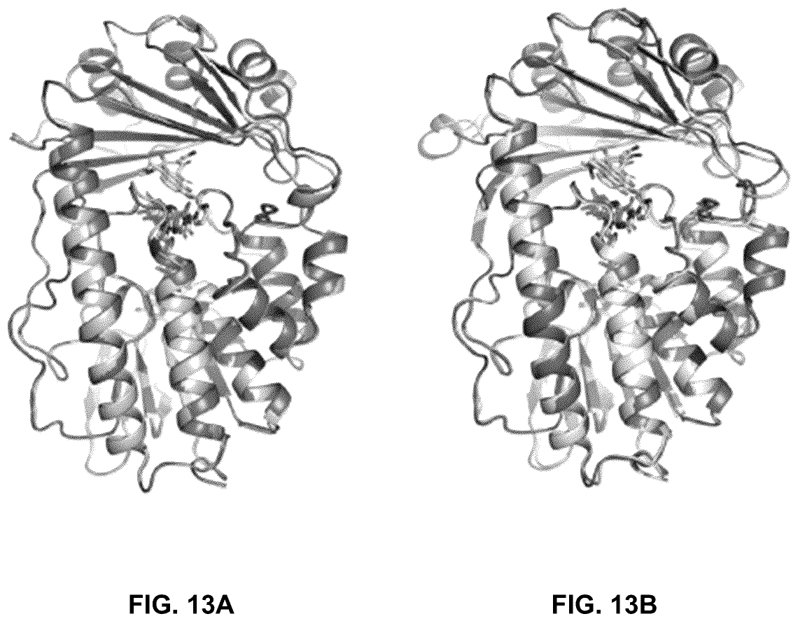

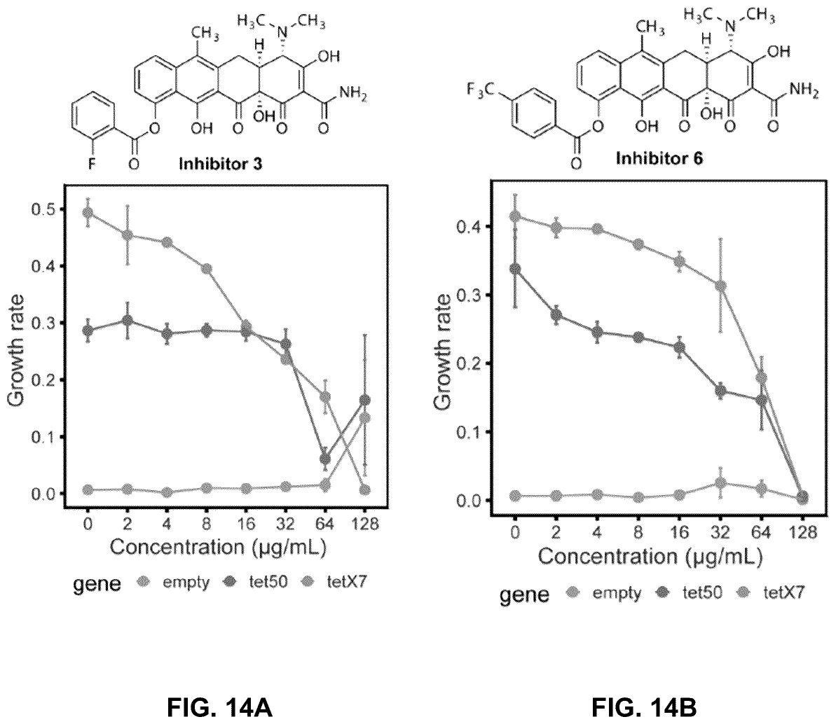

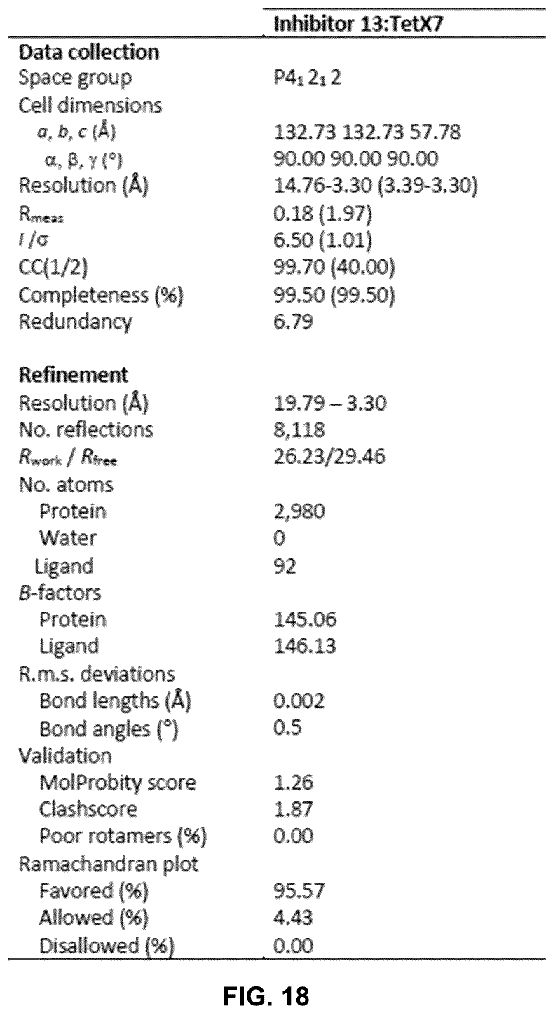

To explore the inhibitor binding mode, the structure of TetX7 was solved when cocrystallized with inhibitor 13 at 3.3 Å (FIG. 4A, FIG. 18). The inhibitor-bound TetX7 structure was similar to the inhibitor-free TetX7 structure PDB: 6WG9 with a root-mean-square deviation (r.m.s.d.) of 0.35 Å (FIG. 13A, FIG. 13B). After modeling TetX7 and FAD in the electron density map, a large piece of unmodeled positive density was found next to the isoalloxazine group of the bound FAD cofactor (FIG. 4B). Inhibitor 13 was therefore modeled in with the A-ring component of inhibitor 13 pointing into the substrate-binding pocket and the D-ring component of inhibitor 13 pointing outside, providing space to accommodate the C10-ester present on the D-ring. The aTC component of inhibitor 13 fit well into the map, but no electron density was observed on the C10-ester aryl side chain after refinement, suggesting flexibility. The binding mode of the bound inhibitor 13 was similar to that of aTC bound to TetX6 PDB: 8ER0 (FIG. 4C).

Both aTC and analog 13 can be oxidized by TetX7 while maintaining the inhibition of TC oxidation. Based on LC-MS and optical absorbance spectra, aTC is oxidized at C11a, whereas analog 13 is oxidized at a position that does not disrupt the conjugation of the pi-electron system in the core of the aTC scaffold (FIG. 2A, FIG. 2B, FIG. 2C, FIG. 2D, FIG. 11A, FIG. 11B, FIG. 11C, FIG. 11D, FIG. 11E, FIG. 11F, FIG. 11G, FIG. 11H, FIG. 11I, FIG. 11J, FIG. 11K). The core aTC scaffold of inhibitor 13 forms contacts with the conserved active site residues Q203 and R224, but the C10-pyrimidine-2-carboxylate side chain was too dynamic for observation in the X-ray crystal structure (FIG. 4C). Despite the proven stability of the C10-esters toward various buffers and cell lysates (FIG. 9D), it cannot be ruled out dynamic in crystallo hydrolysis and/or selective trapping of the hydrolysis product aTC during the cocrystallization process. Therefore, a molecular docking with AutoDock Vina was used to model the aryl ester side chain to show that there is ample space in the open TetX7 active site to accommodate this group in multiple conformations. In this binding conformation, the C10-benzoate side chain is too far away from the FAD C4a position, while the two known TC oxidation sites, C11a and C1, are still within striking distance of an FAD C4a-peroxy group. In the aTC:TetX6 structure the distances are 5.7 Å for C11a-C4a and 6.0 Å for C1-C4a. In the new structure of inhibitor 13:TetX7 reported here the distances are 5.3 Å for C11a-C4a and 6.7 Å for C1-C4a. It is noteworthy that the C11a position in the two structures are offset by 1.1 Å, and the C10 atom in the analog 13:TetX7 structure overlays best with the C11a position in the aTC:TetX6 structure. Further, the C10-ester is an electron withdrawing group and may act to decrease the nucleophilicity of the conjugated C11a enol carbon, increasing the transition state energy for C11a hydroxylation by a C4a-peroxy flavin. This subtle shift in conformation and redox potential may account for the observation that aTC, but not the C10-ester aTC analogs, are oxidized at C11a by TetX7 and other Type 1 TDases. The crystal structure of inhibitor 13 bound to TetX7 suggests that the C10-benzoate ester derivatives of aTC still bind tightly to the Type 1 TDase active site and accommodate a wide variety of C10 ester substituents because the placement of this side chain pointing into the spacious opening of the solvent exposed active site.

Tetracycline Rescue in Resistant E. coli, P. Aeruginosa, and M. smegmatis

The ability of the compounds to rescue TC antibacterial activity was evaluated against E. coli cells expressing plasmid-encoded TetX7 or Tet50. The measured MIC value of TC against E. coli expressing TetX7 and Tet50 is >8 μg/mL, so combined MIC studies were performed using variable inhibitor concentrations with a fixed TC concentration of 8 μg/mL to screen for inhibitors that could potentially have synergistic effects with TC against TetX7 and Tet50. Hence, the recorded MIC values reflect the concentration of inhibitor needed to rescue TC antibacterial activity at 8 μg/mL. It was first tested for the inherent antibacterial activity of the inhibitors by performing standard MICs on the inhibitor series. It was found that inhibitors 2-6, 8, and 16-23 displayed no growth inhibitory activity alone at concentrations >128 μg/mL. Inhibitors 7 and 9-15 inhibited growth to some extent, with MIC values ranging from 8 to 64 μg/mL. The inhibitors rescued the activity of TC to varying degrees, with MICs as low as 2 μg/mL for compounds 7, 10, 11, and 15 when combined with a fixed concentration of TC at 8 μg/mL (Table 3). These data are consistent with TDase inhibitors rescuing TC activity, because even inhibitors that lack inherent antibacterial activity (compound 8, combined MIC=4 μg/mL) have similar rescue activity as inhibitors that are antibacterial (compound 7, combined MIC=2 μg/mL).

TABLE 3
Minimum Inhibitory Concentration (MIC in μg/mL) against E. coli DH5αZ1
Expressing TetX7 or Tet50 from pZE24 Plasmid in the Absence or Presence of 8 μg/mL
TC

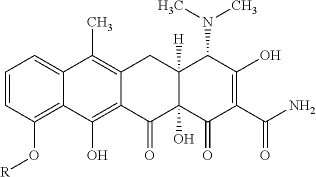

C10-Benzoate Ester, Formula (I)

| | Inhibitor | Empty | | TetX7 | | Tet50 | |
|---|---|---|---|---|---|---|---|
| R group | # | No TC | TC | No TC | TC | No TC | TC |
| 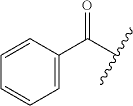 [SMILES: *C(=O)c1ccccc1] | 2 | >128 | 0 | >128 | >128 | >128 | >128 |
| 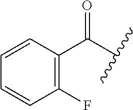 [SMILES: *C(=O)c1ccccc1F] | 3 | >128 | 0 | >128 | 128 | >128 | >128 |
| 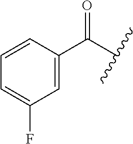 [SMILES: *C(=O)c1cccc(F)c1] | 4 | >128 | 0 | >128 | >128 | >128 | >128 |
| 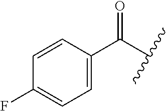 [SMILES: *C(=O)c1ccc(F)cc1] | 5 | >128 | 0 | >128 | >128 | >128 | >128 |
| 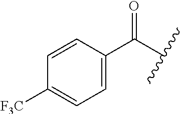 [SMILES: *C(=O)c1ccc(C(F)(F)F)cc1] | 6 | >128 | 0 | >128 | 128 | >128 | 128 |
| 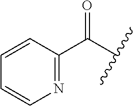 [SMILES: *C(=O)c1ccccn1] | 7 | 16 | 0 | 16 | 2 | 16 | 2 |
| 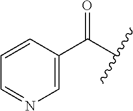 [SMILES: *C(=O)c1cccnc1] | 8 | >128 | 0 | >128 | 8 | >128 | 4 |
| 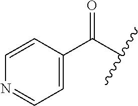 [SMILES: *C(=O)c1ccncc1] | 9 | 64 | 0 | 64 | 32 | 64 | 8 |
| 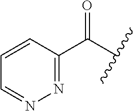 [SMILES: *C(=O)c1cccnn1] | 10 | 16 | 0 | 16 | 4 | 16 | 2 |
| 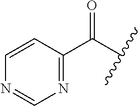 [SMILES: *C(=O)c1ccncn1] | 11 | 16 | 0 | 16 | 8 | 16 | 2 |
| 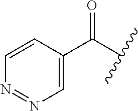 [SMILES: *C(=O)c1ccnnc1] | 12 | 16 | 0 | 16 | 8 | 16 | 4 |
| 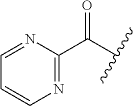 [SMILES: *C(=O)c1ncccn1] | 13 | 8 | 0 | 16 | 4 | 16 | 4 |
| 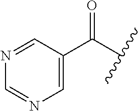 [SMILES: *C(=O)c1cncnc1] | 14 | 32 | 0 | 16 | 8 | 32 | 8 |
| 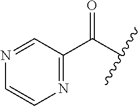 [SMILES: *C(=O)c1cnccn1] | 15 | 32 | 0 | 16 | 8 | 16 | 2 |

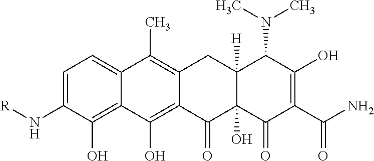

C9-Benzamide, Formula (II)

| | Inhibitor | Empty | | TetX7 | | Tet50 | |
|---|---|---|---|---|---|---|---|
| R group | # | No TC | TC | No TC | TC | No TC | TC |
| 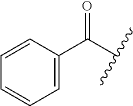 [SMILES: *C(=O)c1ccccc1] | Previous Work | >128 | 0 | >128 | >128 | >128 | >128 |
| 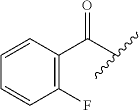 [SMILES: *C(=O)c1ccccc1F] | Previous Work | >128 | 0 | >128 | 128 | >128 | >128 |
| 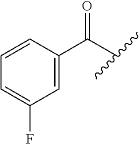 [SMILES: *C(=O)c1cccc(F)c1] | Previous Work | >128 | 0 | >128 | >128 | >128 | >128 |
| 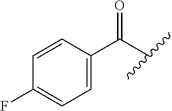 [SMILES: *C(=O)c1ccc(F)cc1] | NA | — | — | — | — | — | — |
| 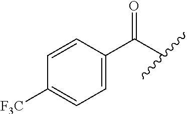 [SMILES: *C(=O)c1ccc(C(F)(F)F)cc1] | NA | — | — | — | — | — | — |
| 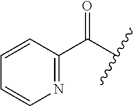 [SMILES: *C(=O)c1ccccn1] | 16 | >128 | 0 | >128 | >128 | >128 | >128 |
| 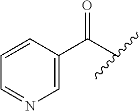 [SMILES: *C(=O)c1cccnc1] | 17 | >128 | 0 | >128 | >128 | >128 | >128 |
| 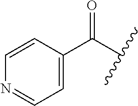 [SMILES: *C(=O)c1ccncc1] | 18 | >128 | 0 | >128 | >128 | >128 | >128 |
| 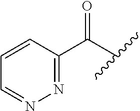 [SMILES: *C(=O)c1cccnn1] | 19 | >128 | 0 | >128 | 8 | >128 | >128 |
| 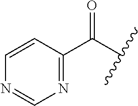 [SMILES: *C(=O)c1ccncn1] | 20 | >128 | 0 | >128 | 8 | >128 | >128 |
| 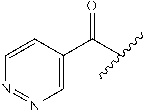 [SMILES: *C(=O)c1ccnnc1] | 21 | >128 | 0 | >128 | >128 | >128 | >128 |
| 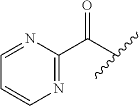 [SMILES: *C(=O)c1ncccn1] | 22 | >128 | 0 | >128 | 16 | >128 | 64 |
| 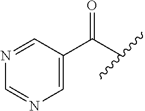 [SMILES: *C(=O)c1cncnc1] | NA | — | — | — | — | — | — |
| 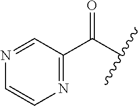 [SMILES: *C(=O)c1cnccn1] | 23 | >128 | 0 | >128 | >128 | >128 | >128 |

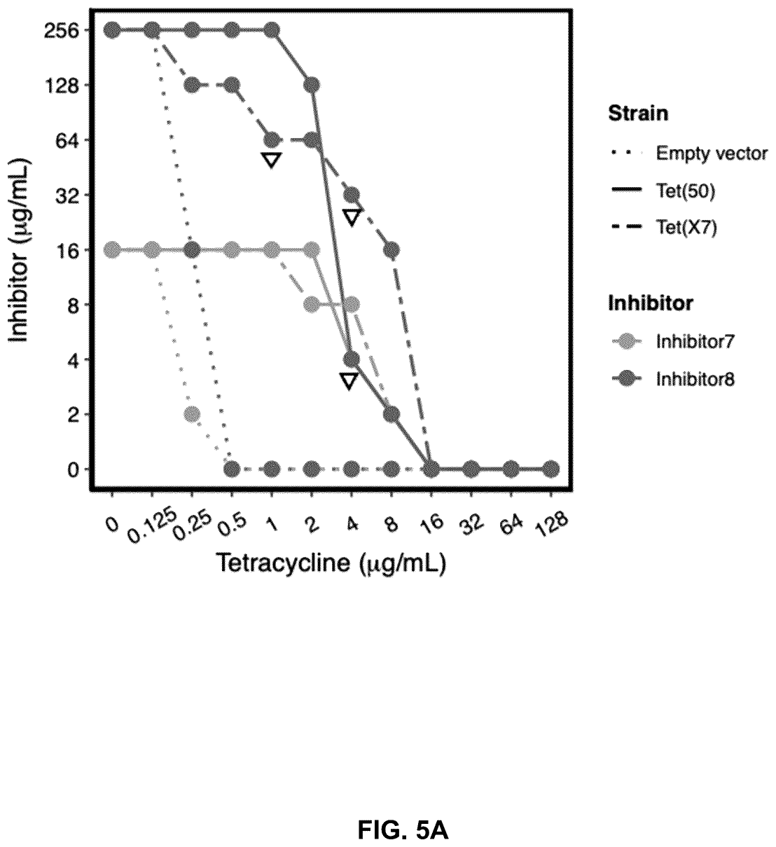

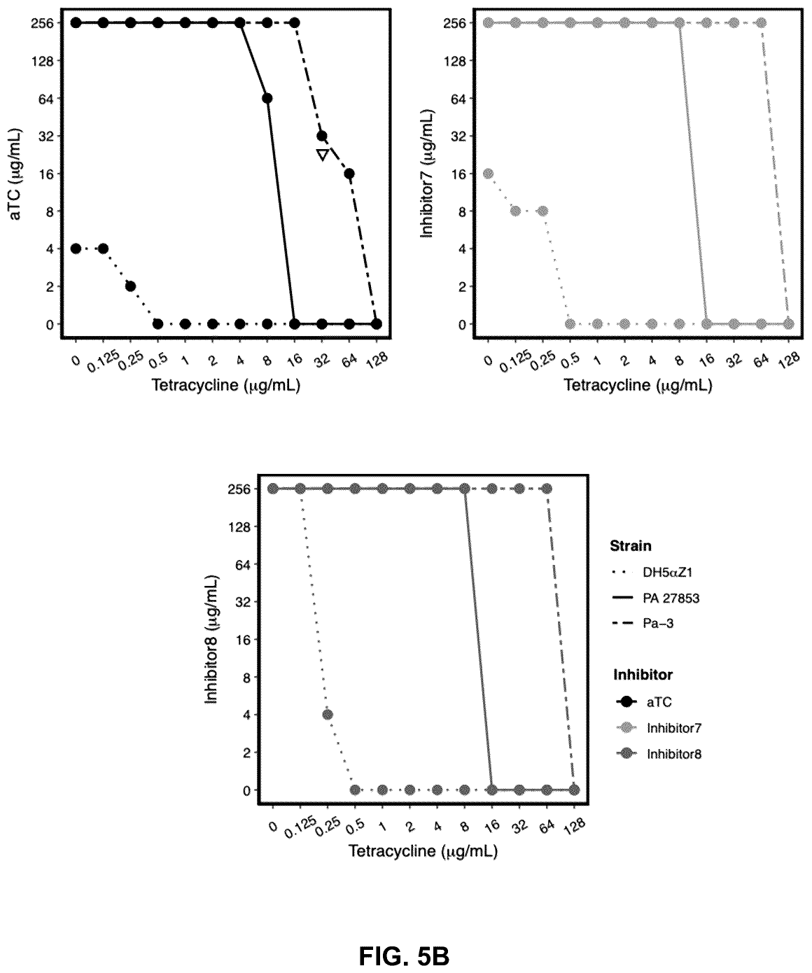

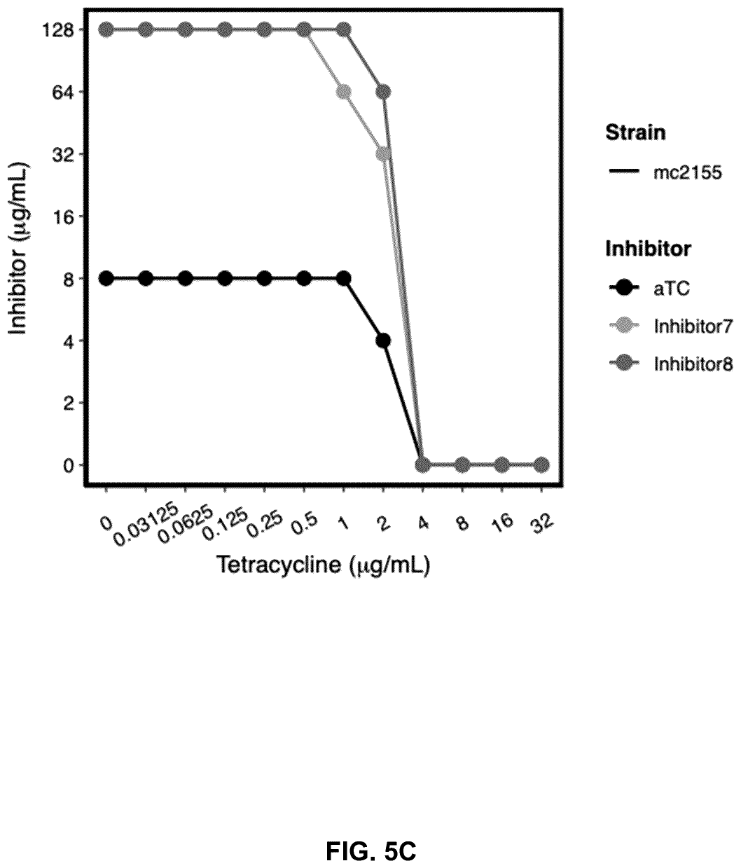

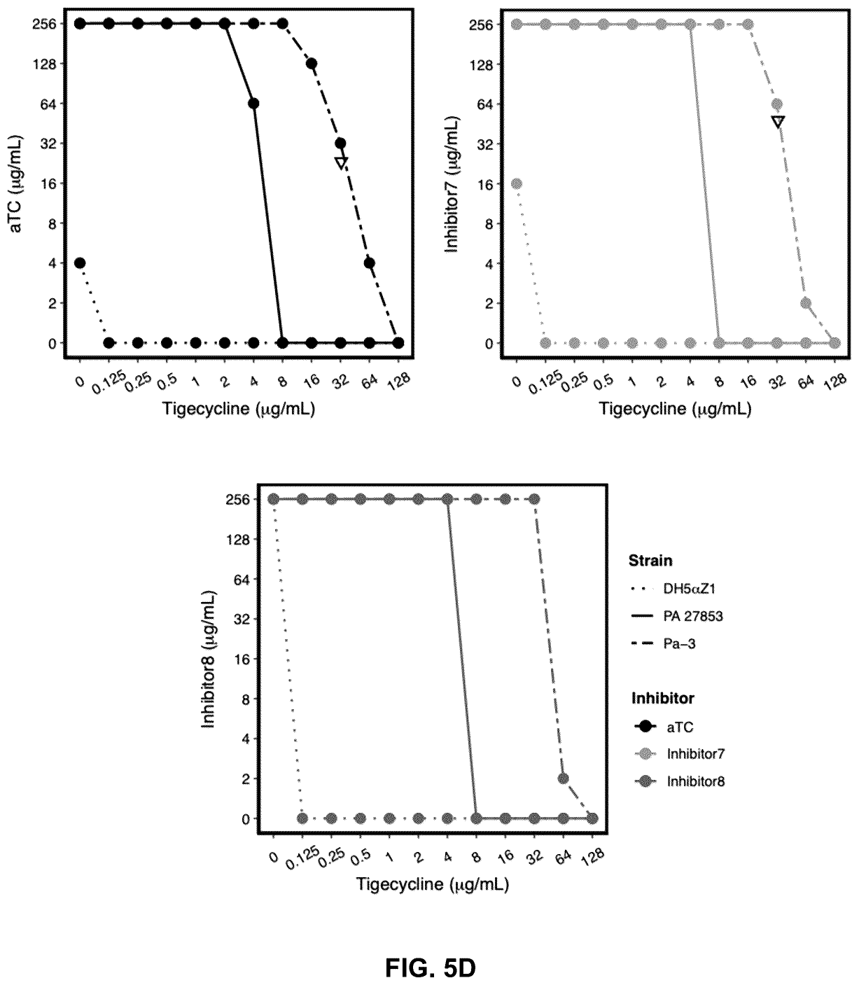

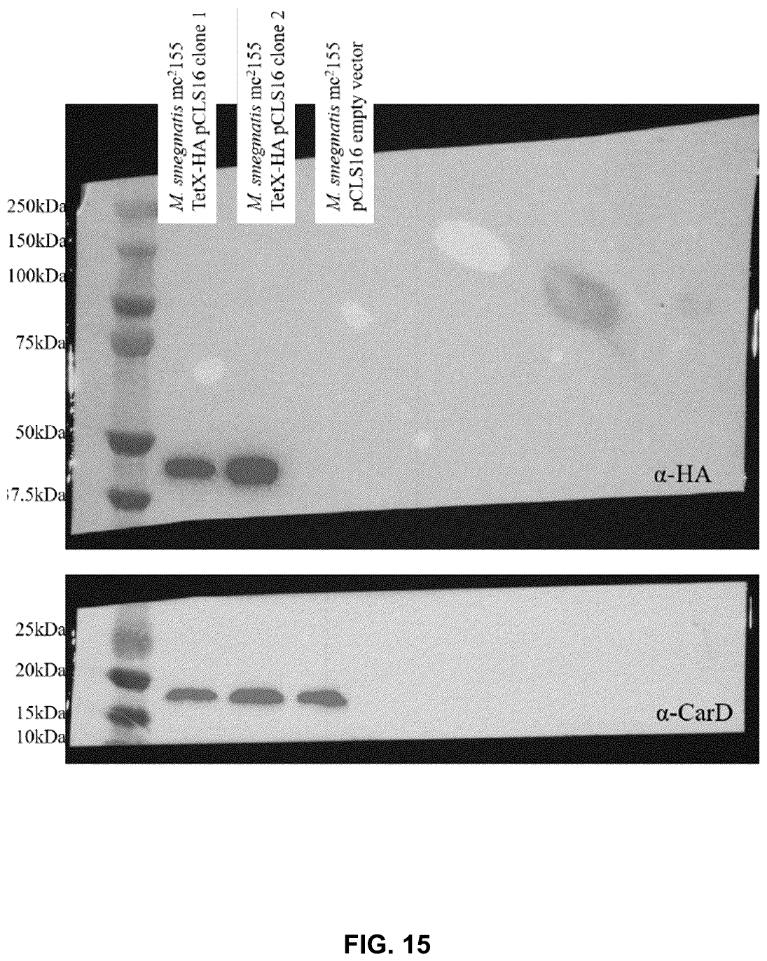

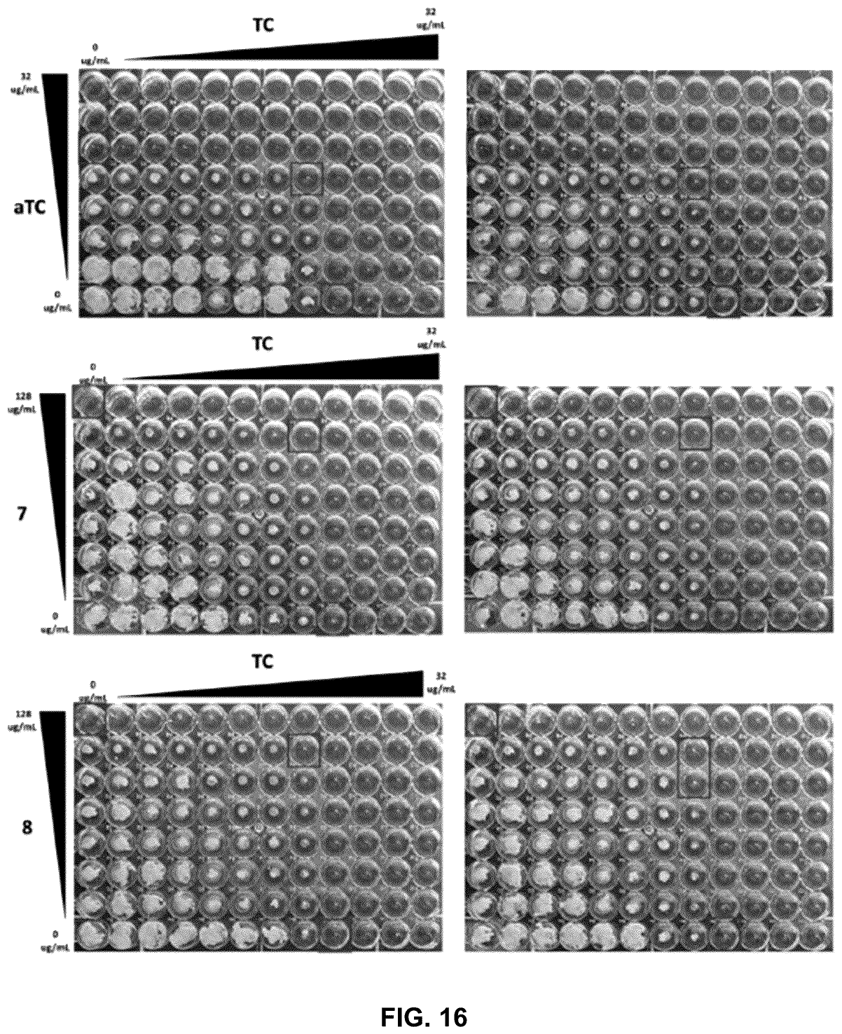

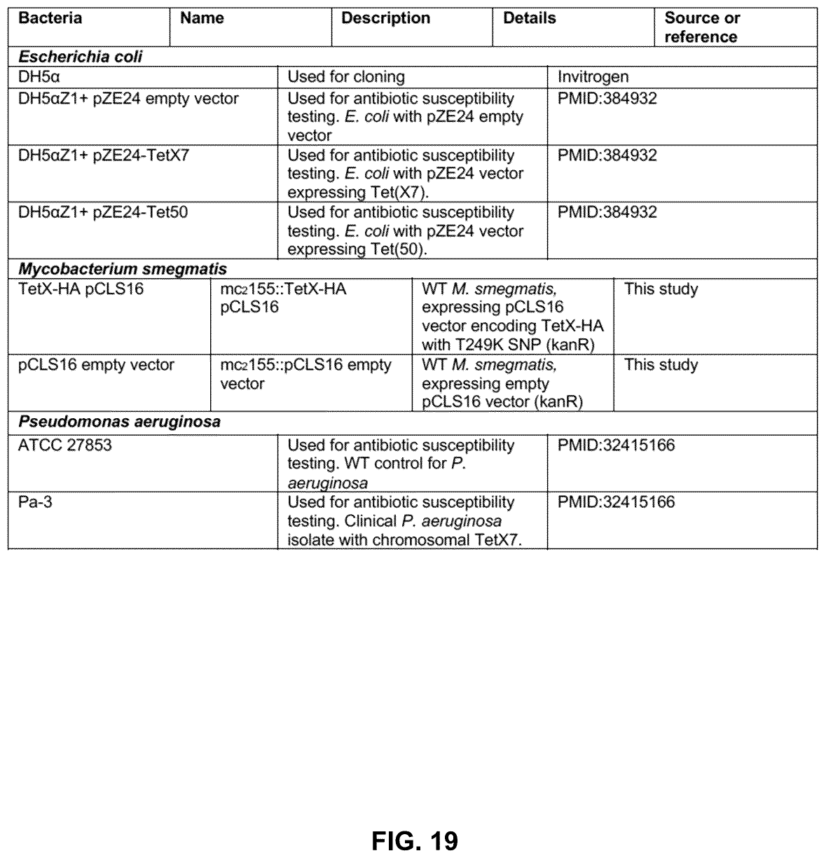

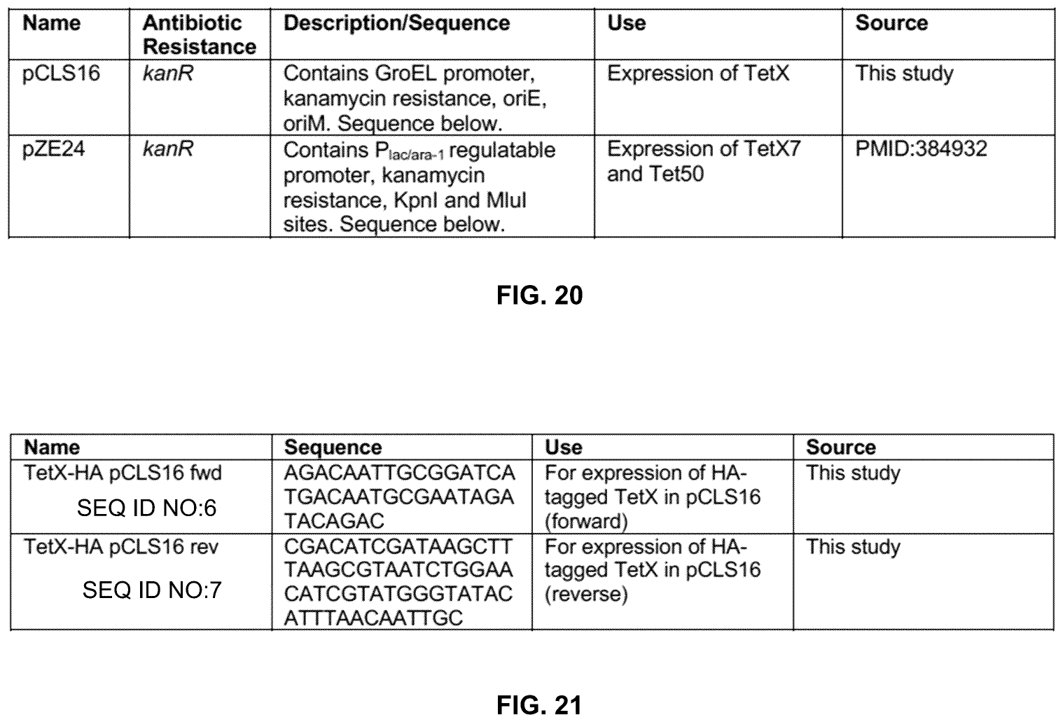

In previous work, the growth rate analysis was used to reveal synergy between C9-benzamide aTC derivatives and TC against E. coli expressing TetX7 or Tet50. The growth rate analysis enabled us to observe synergy even when growth was only partially inhibited. This type of growth rate analysis was performed for the C10-ester aTC derivatives 3 and 6 and observed complete growth inhibition of E. coli at 128 μg/mL inhibitor and 8 μg/mL TC (FIG. 12). Next, a checkerboard MIC assays was used to determine the fractional inhibitory concentration index (FICI) against strains of E. coli, P. aeruginosa, and M. smegmatis. Three inhibitors were selected representing compounds with or without inherent antibacterial activity against E. coli (Table 3): antibacterial aTC (1), antibacterial C10-ester (7), and nonantibacterial C10-ester (8). Interestingly, the only structural difference between inhibitors 7 and 8 is the position of the nitrogen in the pyridine ring of the aryl acid side chain. It was determined FICI scores for inhibitors 1, 7, and 8 in combination with TC or Tig against a panel of bacteria representing laboratory strains (E. coli DH5aZ1; M. smegmatis mc²155) and clinical isolates (P. aeruginosa PA27853 and PA-3) with varying degrees and mechanisms of TC resistance (FIG. 5A, FIG. 5B, FIG. 5C, FIG. 5D, FIG. 19, FIG. 20, FIG. 21, Table 4). aTC (1) was synergistic with TC and Tig against P. aeruginosa PA-3 (FICI≤0.5) and was indifferent or additive against all other strains tested. Inhibitor 7 was synergistic with Tig against P. aeruginosa PA-3 (FICI≤0.5) and was indifferent or additive against all other strains tested. Inhibitor 8 was synergistic with E. coli DH5αZ1 expressing TetX7 (FICI=0.31-0.56) or Tet50 (FICI=0.265-0.625) and was indifferent or additive against all other strains tested. Inhibitor 8 had a borderline synergistic effect with Tig against P. aeruginosa PA-3, but since this inhibitor lacks inherent antibacterial activity, it was not possible to distinguish the FICI=0.5 as a reflection of compound synergy. All three inhibitors produced additive FICI values ≤1 with borderline synergy in some cases against M. smegmatis mc²155 expressing TetX as a fusion with hemagglutinin (FIG. 15), although the visual scoring of the growth phenotypes showed synergy when partial growth inhibition was considered (FIG. 16).

TABLE 4
Fractional Inhibitory Concentration Index (FICI) of aTC, Inhibitor 7, and
Inhibitor 8 against E. coli, P. aeruginosa, and M. smegmatis

| | | | | fold |
|---|---|---|---|---|
| | drug | | | increase in |
| strain | combination | FICI | effect | TC MIC |
| E. coli DH5αZ1 + | tetracycline - | 1.12 | indifferent | NA |
| pZE24 | inhibitor 7 | 5 | | |
| | tetracycline - | 1 | indifferent | NA |
| | inhibitor 8 | | | |
| E. coli DH5αZ1 + | tetracycline - | 0.62 | additive | 2 |
| pZE24-TetX7 | inhibitor 7 | | | |
| | tetracycline - | 0.37 | synergetic | 8 |
| | inhibitor 8 | 5 | | |
| E. coli | tetracycline - | 0.62 | additive | 2 |
| DH5αZ1 + pZE24- | inhibitor 7 | 5 | | |
| Tet50 | tetracycline - | 0.26 | synergetic | 4 |
| | inhibitor 8 | 5 | | |
| E. coli DH5αZ1 | tetracycline-aTC | >1 | indifferent | NA |
| | tetracycline - | 0.75 | additive | 4 |
| | inhibitor 7 | | | |
| | tetracycline - | >1 | indifferent | NA |
| | inhibitor 8 | | | |
| | tigecycline-aTC | >1 | indifferent | NA |
| | tigecycline - | >1 | indifferent | NA |
| | inhibitor 7 | | | |
| | tigecycline - | >1 | indifferent | NA |
| | inhibitor 8 | | | |
| P. aeruginosa PA | tetracycline-aTC | 0.75 | additive | 2 |
| 27853 | tetracycline - | >1 | indifferent | NA |
| | inhibitor 7 | | | |
| | tetracycline - | >1 | indifferent | NA |
| | inhibitor 8 | | | |
| | tigecycline-aTC | 0.75 | additive | 4 |
| | tigecycline - | >1 | indifferent | NA |
| | inhibitor 7 | | | |
| | tigecycline - | >1 | indifferent | NA |
| | inhibitor 8 | | | |
| P. aeruginosaPa3⁻ | tetracycline-aTC | 0.37 | synergetic | 4 |
| | | 5 | | |
| | tetracycline - | 1 | indifferent | NA |
| | inhibitor 7 | | | |
| | tetracycline - | >1 | indifferent | NA |
| | inhibitor 8 | | | |
| | tigecycline-aTC | 0.37 | synergetic | 4 |
| | | 5 | | |
| | tigecycline - | 0.5 | synergetic | 4 |
| | inhibitor 7 | | | |
| | tigecycline - | 0.50 | additive | 2 |
| | inhibitor 8 | 7 | | |
| M. smegmatis mc²155 | tetracycline-aTC | 1 | indifferent | 2 |
| | tetracycline - | 0.75 | additive/ | 2 |
| | inhibitor 7 | −1 | indifferent | |
| | tetracycline - | 0.75 | additive/ | 2 |
| | inhibitor 8 | −1 | indifferent | |

### CONCLUSIONS

Shown herein was the synthesis and characterization of inhibitors of TDase enzymes via the fusion of a known inhibitor, aTC, and structural mimics of nicotinic acid joined via ester linkages to the C10-phenolate on the D-ring of aTC. While many semisynthetic modifications of tetracyclines have been reported, esterification of C10 has not been explored in depth. Only one report of C10 esterification of tetracyclines was found within the patent literature. This is not surprising given that the C10 phenol group is essential for tetracycline Mg²⁺ chelation and ribosome binding, so there is little motivation to explore SAR at this position for the purpose of inhibiting protein translation. For this reason, the inherent antibacterial activity of the C10-ester and C9-amide aTC analogs is not associated with the inhibition of protein synthesis. aTC is known to disrupt bacterial cell membranes, so it is possible that this mechanism contributes to the observed antibacterial activity and ability of the C10-ester analogs to permeate the bacterial cell envelope and inhibit intracellular TDases. Given that there is growing exploration in the use of TC derivatives in other therapeutic areas, including inflammatory diseases, the simple synthetic method for direct C10 esterification could be broadly applicable in analog production aimed at these drug development efforts.

The prior work on aTC-based TDase inhibitors demonstrated that C9-benzamides and C9-benzylamines are potent bivalent TDase inhibitors that compete with both TC and NADPH for TDase binding. The C10-benzoate ester aTC analogs was designed with this potential for bivalent binding but discovered that these analogs show mixed competitive inhibition with respect to TC and NADPH depending on the nature of the aryl ester side chain. The analog 13:TetX7 cocrystal structure showed that the C10-benzoate ester inhibitor occupies the substrate binding mode. The C10-benzoate side chain is disordered, and the ligand orientation indicates free rotation of this group in the solvent exposed region of the large opening to the active site cavity. Based on molecular modeling, the placement of benzamides at the C9 position of the aTC D-ring allows for the orientation of the D-ring into the active site to interact with the FAD in the “out” conformation. However, at the C10-position, the benzoate ester moiety is sterically occluded from accessing this binding orientation and forces the C10-ester aTC analogs into the substrate binding mode. In this orientation, a wide range of C10-functionalization should be tolerated along with added substituents at the C8 and C9 positions of the aTC D-ring.

Knowing that the inhibitor mechanism and TDase binding mode can guide structure-based drug design, to ultimately drive inhibitor optimization by whole cell efficacy toward the rescue of TC growth inhibitory activity against TDase expressing pathogens. This is the most exciting aspect of the C10-ester aTC analogs, which are more active than C9-benzamides bearing the same aryl acid side chain. This improved activity is attributed to increased water solubility, cell permeability, and inhibition of TDases. The most distinct SAR pattern to emerge was that C10-ester derivatives derived from N-containing heterocyclic aryl acids were the most active compounds in the series. In total, compared to 3/11 C9-benzamides, 11/14 C10-benzoates were active in rescuing TC antibacterial activity based on MIC determination (Table 3). The most potent 9/11 C10-benzoates were derived from N-containing pyridine, pyrimidine, or pyrazine aryl acids. Within the pyridine series, the picolinic and isonicotinic acid analogs 7 and 9, respectively, both showed inherent antibacterial activity while nicotinic acid analog 8 was able to rescue TC activity against both Type 1 and 2 TDases without showing any inherent antibacterial activity; the latter is a true reflection of compound synergy via TDase inhibition. It is noteworthy that both picolinic and isonicotinic acid alone possess antimicrobial activity while nicotinic acid is nontoxic to all cell types. Picolinic acid is a natural metabolite derived from the catabolism of tryptophan, a metal chelator, and a membrane uncoupler. Isonicotinic acid is the core scaffold of the anti-TB drug isoniazid, which inhibits the enoyl-acyl carrier protein reductase InhA resulting in reduced fatty acid biosynthesis. The hydrolysis of picolinic acid esters is promoted by the presence of divalent metal cations such as Co²⁺, Ni²⁺, Zn²⁺, and Cu²⁺, suggesting that the enhanced antibacterial activity of picolinate ester 7 (E. coli MIC=16 μg/mL) relative to isonicotinate ester 9 (E. coli MIC=64 μg/mL) may be due to metal cation-promoted intracellular hydrolysis; however, it's noted that both inhibitors 7 and 8 were stable toward hydrolysis in E. coli cell lysates (FIG. 9A, FIG. 9B, FIG. 9C, FIG. 9D).

It remains unclear if it is more advantageous to employ antibacterial or nonantibacterial TDase inhibitors since both inhibitors 7 and 8 are capable acting synergistically with TC antibiotics against tetracycline-resistant bacteria. It's noted that for beta-lactam antibiotics, beta-lactamase inhibitors are typically nonantibacterial and hence do not apply a separate selection force for resistance. It is also recognize that certain combinations of antibacterial agents can reduce the occurrence of spontaneous resistance. All of the pyrimidine and pyrazine-derived esters 10-15 were antibacterial alone against E. coli and strongly rescued TC activity (Table 3). Pyrimidine and pyrazine carboxylic acids are known inhibitors of fatty acid synthesis. Further, pyrazinamide is a prodrug for pyrazinoic acid used to treat TB. It's noted that pyrimidine and pyrazine-derived esters 10-15 were generally less cytotoxic in the human renal proximal tubule epithelial cell viability assay (FIG. 7A, FIG. 7B, FIG. 7C, FIG. 17). The heterocyclic aryl acid side chain might provide a means to systematically improve TDase binding affinities and redox potentials by exploring pi-pi stabilization energies with the FAD isoalloxazine ring, which has been rigorously explored in FAD-binding RNA aptamers and synthetic flavin model systems. While other TDase inhibitor types have been reported, the aTC based inhibitors are favored due to the proven in vivo efficacy of this general pharmacophore and the ease of analog synthesis through the high-yield one step C10 esterification protocol. While further mechanistic studies and optimization of the C10-ester series are merited, this study supports that C10-ester derivatives of aTC are attractive water-soluble candidates for combination therapy with TC antibiotics to overcome TDase-mediated resistance.

### EXPERIMENTAL METHODS

Synthesis of C10-Benzoate Ester aTC Derivatives (Compounds 2-15) The corresponding benzoic acid (0.0199 g, 0.162 mmol, 1.00 equiv) and DIPEA (0.045 mL, 0.260 mmol, 1.60 equiv) were dissolved in 3.5 mL of anhydrous DMF (DriSolv) in a vial and charged with HATU (0.0617 g, 0.162 mmol, 1.00 equiv). The clear solution turned pale yellow and was stirred at room temperature for 10 min before the addition of compound aTC (HCl salt). The reaction solution was stirred for 5 min at room temperature and concentrated under reduced pressure via rotary evaporation. The residue was dissolved in MeOH, filtered through a 0.45 μm PTFE syringe filter, and purified by RP-C18 prep-HPLC to provide the desired products 2-15 as the corresponding formic acid salts. Compound purity, including epimeric ratio, was assessed by LCMS and NMR (see Example 2). All compounds tested in biological assays were determined to be >95% pure from detectable organic contaminants as a reported ratio of C4-epimers. See Example 2 for synthetic procedures and compound characterization data. All compounds were fully soluble at concentrations tested.

### Synthesis of C9-Benzamide-aTC Derivatives (Compounds 16-23)

C9-substituted aTC derivatives were synthesized following a general protocol briefly described herein. Compound purity, including epimeric ratio, was assessed by LCMS and NMR as reported in Example 2. All compounds tested in biological assays were determined to be >95% pure from detectable organic contaminants as a reported ratio of C4-epimers. See Example 2 for synthetic procedures and compound characterization data. All compounds were fully soluble at concentrations tested.

### Cloning, Expression, and Purification of TDases

All genes encoding TDases used in this study were cloned and inserted into pET28b(+) vectors (Novagen) as previously described (BamHI and Ndel restriction sites) and transformed into BL21-Star (DE3) competent cells (Life Technologies). The cells were cultured at 37° C. in lysogeny broth (LB) containing kanamycin (Kan) at 0.05 mg/mL (final concentration); once the culture reached an OD600 of ˜0.6, the cells were cooled to 0° C. in an ice water bath. Protein expression was induced by the addition of 1 mM IPTG (final concentration), and the cells were grown at 15° C. for 12-15 h. To harvest protein, the induced cells were pelleted by centrifugation at 4000 rpm for 15 min (4° C.) and resuspended in cold 40 mL of lysis buffer (50 mM K₂HPO₄, 500 mM NaCl, 20 mM imidazole, 10% glycerol, 5 mM BME, pH 8.0) containing SIGMAFAST© protease inhibitor (Millipore-Sigma). The cell suspensions were flash frozen in liquid nitrogen and stored at −80° C. The frozen cell suspensions were thawed and mechanically lysed using an Avestin EmulsiFlex-C5 cell disruptor, and the resulting lysate was clarified via ultracentrifugation at 45,000 rpm for 35 min at 4° C. The clarified supernatant was transferred to a fritted column containing washed and equilibrated Ni-NTA resin and incubated for 30-45 min with gentle rocking. The resin was then washed with lysis buffer (2×40 mL), and the protein was eluted from the resin with elution buffer (5×10 mL elutions, 50 mM K₂HPO₄, 500 mM NaCl, 5 mM BME, 300 mM imidazole, 10% glycerol, pH 8.0). Fractions containing the desired proteins (as determined by SDS-PAGE analysis) were combined and transferred to a 10,000 molecular weight cutoff (MWCO) Snakeskin dialysis tubing (ThermoScientific) and equilibrated in dialysis buffer (50 mM K₂HPO₄ pH 8.0, 150 mM NaCl, 1 mM DTT) overnight. The dialyzed protein solutions were concentrated using a 30,000 MWCO Amicon centrifugal filter (Millipore-Sigma), and the concentrated protein solution was flash frozen as beads in liquid nitrogen (50 μL portions) and stored at −80° C.

### In Vitro Characterization of TDases

TDase reactions were prepared in 100 mM TAPS buffer (pH 8.5) with 252 μM NADPH and 5.04 mM MgCl₂, 40 μM substrate and 0.4 μM TDase enzyme (TetX7 and Tet50) (all concentrations represent final working concentrations). Reaction progress was monitored by optical absorbance spectroscopy (280-550 nm, 1 nm and 5 min intervals) over 2 h. Twenty μL aliquot of of reaction mixture was removed at 0, 5, 30, 60, 90, and 120 min time points and used for colorimetric detection of hydrogen peroxide formation performed using an aqueous Pierce Quantitative Peroxide Assay Kit (ThermoScientific). Each 20 μL aliquot (performed in triplicate on three separate aliquots for each time point) was added to a 96-well plate containing 200 μL of working reagent (prepared according to specifications for Pierce Quantitative Peroxide Assay kit). The plate was incubated for at least 20 min at room temp and observed for color change.

### Cytotoxicity

All cytotoxicity studies were performed by Eurofins Panlabs (St. Charles, MO). Cell viability of Human Renal Proximal Tubule Epithelial Cells was determined using CellTiter-Glo after 48 h incubation at 37° C. The percent of control was calculated using the formula: Control (%)=(compound/T1)*100 where “compound” is the individual reading in the presence of the test compound and T1 is the mean reading in the absence of the test compound. The percent of inhibition is calculated by subtracting the percent of control from 100. The IC₅₀ value (concentration causing a half-maximal inhibition of the control value) was determined by nonlinear regression analysis of the concentration-response curve using the Hill equation. The positive control was staurosporine and all tests were performed in duplicate as independent trials.

### Measuring Growth Rate

Overnight cultures were grown in fresh MH-II+KAN50 broth supplemented with 1 mM IPTG to exponential phase (OD600=0.3-0.8), then diluted to OD600=0.1, and inoculated into each 96-well panel at a 1:1 ratio. To avoid edge effects only the interior wells included cells, and the exterior wells were filled with cell-free broth. Plates were sealed with Breathe-Easy membranes (Sigma-Aldrich) then incubated at 37° C. with continuous shaking and OD600 measurements taken every 5 min for 20 h using a Synergy H1 plate reader (BioTek). Maximal growth rate was calculated from this plate reader data using GrowthRateR. This function log-transforms growth curves and generates a rolling regression with a shifting window of 1 h, such that the maximum slope of any of the regressions is the maximal growth rate.

### Antibiotic Susceptibility Testing—Minimum Inhibitory Concentration (MIC)

Inhibitor screening by antibiotic susceptibility tests (AST) were performed with the microbroth dilution method, following CLSI guidelines and as previously described. MIC panels were prepared in 96-well flat-bottom microplates (Corning) by 2-fold serial dilution of inhibitors in cation-adjusted MH-II broth (BD) supplemented with 50 μg/mL kanamycin (KAN50), 1 mM IPTG, and with or without 16 μg/mL of tetracycline (TC). The panels were stored at −80° C. before use. Single colonies of E. coli DH5aZ1 containing TetX7, Tet50, or empty vector in pZE24 expression system were grown in MH-II broth with KAN50 overnight at 37° C. On the day of the experiment, MIC panels were thawed at room temperature. Overnight cultures were subcultured in fresh MH-II broth with KAN50 and 1 mM IPTG, grown to exponential phase (OD600 of 0.3-0.8), then diluted in MH-II broth with KAN50 and 1 mM IPTG. The diluted cells were inoculated into the MIC panel at a 1:1 ratio, resulting in a final concentration of ˜5×10⁵ CFU/mL cells in each well. The panels were incubated at 37° C. for 20 h, then scored by visual inspection. Potential synergistic effects between inhibitors and TC were evaluated by comparing the MIC of inhibitors alone to the MIC of inhibitors in combination with TC. Each test was performed in triplicate, with no-antibiotic and no-cell control wells.

### Antibiotic Susceptibility Testing—Fractional Inhibitory Concentration (FICI) Index

The synergistic effects and fractional inhibitory concentration index (FICI) between tetracycline family antibiotics and inhibitors were assessed using a checkerboard assay. FIC panels were prepared in 96-well flat-bottom microplates by performing 2-fold serial dilutions of tetracyclines and inhibitors in cation-adjusted MH-II broth, supplemented with or without KAN50 and 1 mM IPTG. Column 1 and row H contained only the inhibitors or tetracyclines, respectively. The procedures described in the MIC section were followed to prepare and inoculate cells into each well. For E. coli DH5αZ1 strains with TetX7 and Tet50, FIC panels included 1 mM IPTG and KAN50. For Pa3̅ and PA27853, FIC panels were prepared without 1 mM IPTG and KAN50. After cell inoculation, the panels were incubated at 37° C. for 20 h and then scored by visual inspection. FICIs were calculated using the formula: FICI=(A/MIC_{A})+(B/MIC_{B}), where A and B represent the MICs of drugs tested in combination. MIC_{A} and MIC_{B} represent the MICs of drugs when tested alone. Each test was repeated in triplicate and included controls.

### Bacterial Strains, Plasmids, and Oligonucleotides Used

All bacterial strains, plasmids, and oligonucleotides used herein are listed in FIG. 19, FIG. 20, and FIG. 21. The E. coli strains utilized were same as the ones previously described. In brief, TetX7 and Tet50 sequences were cloned into the Kpnl and MIul sites of the pZE24 plasmid (Expressys) and transformed into E. coli DH5αZ1 (Expressys). The Pseudomonas aeruginosa strains Pa3̅ and ATCC 27853 were the same as those described in a previous study, with Pa3̅ containing chromosomal TetX7 genes and ATCC 27853 serving as the P. aeruginosa control lacking TetX7.

Production of M. smegmatis TetX^{T249K}-HA Expressing Cells

B. fragilis TDase TetX with a C-terminal HA tag (TetX-HA) gene was cloned into the pCLS16 vector using InFusion master mix (FIG. 20, FIG. 21) and transformed into E. coli DH5α cells. Sequence of vector was confirmed using Plasmidsaurus sequencing. TetX-HA pCLS16 vector or empty pCLS16 vector were then transformed into M. smegmatis mc²155 cells and expression of gene was confirmed via HA Western blot (FIG. 15). E. coli strains were grown at 37° C. in LB broth supplemented with antibiotics when necessary. M. smegmatis strains were grown at 37° C. in LB broth supplemented with 0.5% dextrose, 0.5% glycerol, and 0.05% Tween-80, and supplemented with antibiotics when necessary (FIG. 19).

### Cloning, Expression, and Purification of TetX for Crystallization

The sequence of TetX7 (GenBank CP025402.1) was cloned and inserted into pET28 with 6His tagged to the N-terminus. The plasmid was transformed into BL21(DE3) (ThermoFisher) and the cells were grown at 37° C. in LB broth supplemented with 50 μg/mL kanamycin. Expression was induced at OD600˜1 with a final concentration of 1 mM IPTG. The cells were harvested 4 h after induction at 37° C. The cell pellet was resuspended in 25 mM Tris, pH 7.4, 0.3 M NaCl, 5 mM DTT and supplemented with Pierce protease inhibitor cocktail (ThermoFisher) and DNase I. The cell suspension was then lysed by sonication for 5 min on ice. Cell debris was removed by centrifugation at 39,000×g for 20 min at 4° C. The supernatant was loaded into Excel Ni-NTA (Cytiva) equilibrated with 25 mM Tris, pH 7.4, 0.3 M NaCl, 5 mM DTT, 30 mM imidazole. The column was then washed with the same buffer. TDase protein was eluted using 25 mM Tris, pH 7.4, 0.3 M NaCl, 5 mM DTT, 150 mM imidazole. Fractions were concentrated and injected into a Superdex 75 Increase column (Cytiva) equilibrated with 20 mM Tris, pH 8.0, 100 mM NaCl, 5 mM DTT for size exclusion chromatography. The fractions were again concentrated and stored at −80° C.

### Crystallization, Data Collection, and Structure Determination of the TetX7-Inhibitor 13 Complex

The protein of TetX7 (25 μL of 12 mg/mL) was mixed with 1 μL of inhibitor 13 (100 mM in DMSO) and incubated on ice for 30 min before setting up crystallization trays. Crystals of TetX7-inhibitor 13 were grown using hanging-drop vapor diffusion at 18° C. The complex mixture (12 mg/mL) was mixed with 0.1 M HEPES, pH 7.5, 0.2 M ammonium sulfate, 16% PEG 4000 and 10% isopropanol at a 1:1 ratio for crystallization. The crystals were cryoprotected in a well solution supplemented with 30% PEG 400 prior to flash freezing in liquid nitrogen. X-ray diffraction experiments were carried out at 100 K at the ALS 5.0.2 beamline at the Advanced Light Source at Lawrence Berkeley National Laboratory. The diffraction data were processed with XDS. The structure was determined by molecular replacement in PHASER using TetX7 PDB: 6WG9 as the search model. Iterative model building using COOT, and refinement collection and refinement using PHENIX led to the current model for (Rwork/Rfree of 26.23/29.46). The data collection and refinement statistics are shown in FIG. 18. The structure has been deposited in the Protein Data Bank under accession code 9DRH.

TABLE 5
Plasmid Sequences

| SEQ ID | | |
|---|---|---|
| NO | NAME | SEQUENCE |
| SEQ | pCLS16 | GCTAGCCAACAAAGCGACGTTGTGTCTCAAAATCTCTGATGTTACATTGCACAAGATAAAA |
| ID | | ATATATCATCATGAACAATAAAACTGTCTGCTTACATAAACAGTAATACAAGGGGTGTTATG |
| NO: 1 | | AGCCATATTCAACGGGAAACGTCTTGCTCGAGGCCGCGATTAAATTCCAACATGGATGCTG |
| | | ATTTATATGGGTATAAATGGGCTCGCGATAATGTCGGGCAATCAGGTGCGACAATCTATCG |
| | | CTTGTATGGGAAGCCCCATGCGCCAGAGTTGTTTCTGAAACATGGCAAAGGTAGCGTTGC |
| | | CAATGATGTTACAGATGAGATGGTCAGACTAAACTGGCTGACGGAATTTATGCCTCTTCCG |
| | | ACCATCAAGCATTTTATCCGTACTCCTGATGATGCATGGTTACTCACCACTGCGATCCCCG |
| | | GGAAAACAGCATTCCAGGTATTAGAAGAATATCCTGATTCAGGTGAAAATATTGTTGATGC |
| | | GCTGGCAGTGTTCCTGCGCCGGTTGCATTCGATTCCTGTTTGTAATTGTCCTTTTAACAGC |
| | | GATCGCGTATTTCGTCTCGCTCAGGCGCAATCACGAATGAATAACGGTTTGGTTGATGCGA |
| | | GTGATTTTGATGACGAGCGTAATGGCTGGCCTGTTGAACAAGTCTGGAAAGAAATGCATAA |
| | | TCTTTTGCCATTCTCACCGGATTCAGTCGTCACTCATGGTGATTTCTCACTTGATAACCTTA |
| | | TTTTTGACGAGGGGAAATTAATAGGTTGTATTGATGTTGGACGAGTCGGAATCGCAGACCG |
| | | ATACCAGGATCTTGCCATCCTATGGAACTGCCTCGGTGAGTTTTCTCCTTCATTACAGAAA |
| | | CGGCTTTTTCAAAAATATGGTATTGATAATCCTGATATGAATAAATTGCAGTTTCATTTGATG |
| | | CTCGATGAGTTTTTCTAATCAGAATTGGTTAATTGGTTGTAACACTGGCAGAGCATTACGCT |
| | | GACTTGACGGGACGGCGGCTTTGTTGAATAAATCGAACTTTTGCTGAGTTGAAGGATCAGA |
| | | TCACGCATCTTCCCGACAACGCAGACCGTTCCGTGGCAAAGCAAAAGTTCAAAATCACCAA |
| | | CTGGTCCACCTACAACAAAGCTCTCATCAACCGTGGCTCCCTCACTTTCTGGCTGGATGAT |
| | | GGGGCGATTCAGGCCTGGTATGAGTCAGCAACACCTTCTTCACGAGGCAGACCTCACTAG |
| | | TTCCACTGAGCGTCAGACCCCGTAGAAAAGATCAAAGGATCTTCTTGAGATCCTTTTTTTCT |
| | | GCGCGTAATCTGCTGCTTGCAAACAAAAAAACCACCGCTACCAGCGGTGGTTTGTTTGCC |
| | | GGATCAAGAGCTACCAACTCTTTTTCCGAAGGTAACTGGCTTCAGCAGAGCGCAGATACCA |
| | | AATACTGTCCTTCTAGTGTAGCCGTAGTTAGGCCACCACTTCAAGAACTCTGTAGCACCGC |
| | | CTACATACCTCGCTCTGCTAATCCTGTTACCAGTGGCTGCTGCCAGTGGCGATAAGTCGTG |
| | | TCTTACCGGGTTGGACTCAAGACGATAGTTACCGGATAAGGCGCAGCGGTCGGGCTGAAC |
| | | GGGGGGTTCGTGCACACAGCCCAGCTTGGAGCGAACGACCTACACCGAACTGAGATACC |
| | | TACAGCGTGAGCATTGAGAAAGCGCCACGCTTCCCGAAGGGAGAAAGGCGGACAGGTAT |
| | | CCGGTAAGCGGCAGGGTCGGAACAGGAGAGCGCACGAGGGAGCTTCCAGGGGGAAACG |
| | | CCTGGTATCTTTATAGTCCTGTCGGGTTTCGCCACCTCTGACTTGAGCGTCGATTTTTGTG |
| | | ATGCTCGTCAGGGGGGCGGAGCCTATGGAAAAACGCCAGCAACGCGGCCTTTTTACGGTT |
| | | CCTGGCCTTTTGCTGGCCTTTTGCTCACATGTTCTTTCCTGCGTTATCCCCTGATTCTGTGG |
| | | ATAACCGTATTACCGCCTTTGAGTGAGCTGATACCGCTCGCCGCAGCCGAACGACCGAGC |
| | | GCAACGCGTGAGCCCACCAGCTCCGTAAGTTCGGGTGCTGTGTGGCTCGTACCCGCGCA |
| | | TTCAGGCGGCAGGGGGTCTAACGGGTCTAAGGCGGCGTGTACGGCCGCCACAGCGGCT |
| | | CTTAGCGGCCCGGAAACGTCCTCGAAACGACGCATGTGTTCCTCCTGGTTGGTACAGGTG |
| | | GTTGGGGGTGCTCGGCTGTCGCTGGTGTTTCATCATCAGGGCTCGACGGGAGAGCGGGG |
| | | GAGTGTGCAGTTGTGGGGTGGCCCCTCAGCGAAATATCTGACTTGGAGCTCGTGTCGGAC |
| | | CATACACCGGTGATTAATCGTGGTTTATTATCAAGCGTGAGCCACGTCGCCGACGAATTTG |
| | | AGCAGCTCTGGCTGCCGTACTGGTCCCTGGCAAGCGACGATCTGCTCGAGGGGATCTAC |
| | | CGCCAAAGCCGCGCGTCGGCCCTAGGCCGCCGGTACATCGAGGCGAACCCAACAGCGCT |
| | | GGCAAACCTGCTGGTCGTGGACGTAGACCATCCAGACGCAGCGCTCCGAGCGCTCAGCG |
| | | CCCGGGGGTCCCATCCGCTGCCCAACGCGATCGTGGGCAATCGCGCCAACGGCCACGCA |
| | | CACGCAGTGTGGGCACTCAACGCCCCTGTTCCACGCACCGAATACGCGCGGCGTAAGCC |
| | | GCTCGCATACATGGCGGCGTGCGCCGAAGGCCTTCGGCGCGCCGTCGATGGCGACCGC |
| | | AGTTACTCAGGCCTCATGACCAAAAACCCCGGCCACATCGCCTGGGAAACGGAATGGCTC |
| | | CACTCAGATCTCTACACACTCAGCCACATCGAGGCCGAGCTCGGCGCGAACATGCCACCG |
| | | CCGCGCTGGCGTCAGCAGACCACGTACAAAGCGGCTCCGACGCCGCTAGGGCGGAATTG |
| | | CGCACTGTTCGATTCCGTCAGGTTGTGGGCCTATCTTCCCGCCCTCATGOGGATCTACCT |
| | | GCCGACCCGGAACGTGGACGGACTCGGCCGCGCGATCTATGCCGAGTGCCACGCGCGA |
| | | AACGCCGAATTTCCGTGCAACGACGTGTGTCCCGGACCGCTACCGGACAGCGAGGTCCG |
| | | CGCCATCGCCAACAGCATTTGGCGTTGGATCACAACCAAGTCGCGCATTTGGGCGGACGG |
| | | GATCGTGGTCTACGAGGCCACACTCAGTGCGCGCCATGCGGCCATCTCGCGGAAGGGCG |
| | | CAGCAGCGCGCACGGCGGCGAGCACAGTTGCGCGGCGCGCAAAGTCCGCGTCAGCCAT |
| | | GGAGGCATTGCTATGAGCGACGGCTACAGCGACGGCTACAGCGACGGCTACAACTGGCA |
| | | GCCGACTGTCCGCAAAAAGCGGCGCGTGACCGCCGCCGAAGGCGCTCGAATCACCGGAC |
| | | TATCCGAACGCCACGTCGTCCGGCTCGTGGCGCAGGAACGCAGCGAGTGGTTCGCCGAG |
| | | CAGGCTGCACGCCGCGAACGCATCCGCGCCTATCACGACGACGAGGGCCACTCTTGGCC |
| | | GCAAACGGCCAAACATTTCGGGCTGCATCTGGACACCGTTAAGCGACTCGGCTATCGGGC |
| | | GAGGAAAGAGCGTGCGGCAGAACAGGAAGCGGCTCAAAAGGCCCACAACGAAGCCGACA |
| | | ATCCACCGCTGTTCTAACGCAATTGGGGAGCGGGTGTCGCGGGGGTTCCGTGGGGGGTT |
| | | CCGTTGCAACGGGTCGGACAGGTAAAAGTCCTGGTAGACGCTAGTTTTCTGGTTTGGGCC |
| | | ATGCCTGTCTCGTTGCGTGTTTCGTTGCGTCCGTTTTGAATACCAGCCAGACGAGACGGG |
| | | GTTCTACGAATCTTGGTCGATACCAAGCCATTTCCGCTGAATATCGTGGAGCTCACCGCCA |
| | | GAATCGGTGGTTGTGGTGATGTACGTGGCGAACTCCGTTGTAGTGCTTGTGGTGGCATCC |
| | | GTGGCGCGGCCGCGGTACCAGATCTTTAAATCTAGAGGTGACCACAACGACGCGCCCGC |
| | | TTTGATCGGGGACGTCTGCGGCCGACCATTTACGGGTCTTGTTGTCGTTGGCGGTCATGG |
| | | GCCGAACATACTCACCCGGATCGGAGGGCCGAGGACAAGGTCGAACGAGGGGCATGACC |
| | | CGGTGCGGGGCTTCTTGCACTCGGCATAGGCGAGTGCTAAGAATAACGTTGGCACTCGC |
| | | GACCGGTGAGTCGTAGGTCGGGACGGTGAGGCCAGGCCCGTCGTCGCAGCGAGTGGCA |
| | | GCGAGGACAACTTGAGCCGTCCGTCGCGGGCACTGCGCCCGGCCAGCGTAAGTAGCGG |
| | | GGTTGCCGTCACCCGGTGACCCCCGGTTTCATCCCCGATCCGGAGGAATCACTTCGATAT |
| | | CGCCAAGACAATTGCGGATCCAGCTGCAGAATTCGAAGCTTATCGATGTCGACGTAGTTAA |
| | | CTAGCGTACGATCGACTGCCAGGCATCAAATAAAACGAAAGGCTCAGTCGAAAGACTGGG |
| | | CCTTTCGTTTTATCTGTTGTTTGTCCGGCCATCATGGCCGCGGTGATCA |
| SEQ | pCLS16 | AAAAAAACCACCGCTACCAGCGGTGGTTTGTTTGCCGGATCAAGAGCTACCAACT |
| ID | TetX-HA | CTTTTTCCGAAGGTAACTGGCTTCAGCAGAGCGCAGATACCAAATACTGTCCTTCTAGTGT |
| NO: 2 | | AGCCGTAGTTAGGCCACCACTTCAAGAACTCTGTAGCACCGCCTACATACCTCGCTCTGCT |
| | | AATCCTGTTACCAGTGGCTGCTGCCAGTGGCGATAAGTCGTGTCTTACCGGGTTGGACTC |
| | | AAGACGATAGTTACCGGATAAGGCGCAGCGGTCGGGCTGAACGGGGGGTTCGTGCACAC |
| | | AGCCCAGCTTGGAGCGAACGACCTACACCGAACTGAGATACCTACAGCGTGAGCTATGAG |
| | | AAAGCGCCACGCTTCCCGAGGGGAGAAAGGCGGACAGGTATCCGGTAAGCGGCAGGGTC |
| | | GGAACAGGAGAGCGCACGAGGGAGCTTCCAGGGGGAAACGCCTGGTATCTTTATAGTCC |
| | | TGTCGGGTTTCGCCACCTCTGACTTGAGCGTCGATTTTTGTGATGCTCGTCAGGGGGGCG |
| | | GAGCCTATGGAAAAACGCCAGCAACGCGGCCTTTTTACGGTTCCTGGCCTTTTGCTGGCC |
| | | TTTTGCTCACATGTTCTTTCCTGCGTTATCCCCTGATTCTGTGGATAACCGTATTACCGCCT |
| | | TTGAGTGAGCTGATACCGCTCGCCGCAGCCGAACGACCGAGCGCAACGCGTGAGCCCAC |
| | | CAGCTCCGTAAGTTCGGGCGCTGTGTGGCTCGTACCCGCGCATTCAGGCGGCAGGGGGT |
| | | CTAACGGGTCTAAGGCGGCGTGTACGGCCGCCACAGCGGCTCTCAGCGGCCCGGAAACG |
| | | TCCTCGAAACGACGCATGTGTTCCTCCTGGTTGGTACAGGTGGTTGGGGGTGCTCGGCTG |
| | | TCGCTGGTGTTCCACCACCAGGGCTCGACGGGAGAGCGGGGGAGTGTGCAGTTGTGGG |
| | | GTGGCCCCTCAGCGAAATATCTGACTTGGAGCTCGTGTCGGACCATACACCGGTGATTAA |
| | | TCGTGGTCTACTACCAAGCGTGAGCCACGTCGCCGACGAATTTGAGCAGCTCTGGCTGCC |
| | | GTACTGGCCGCTGGCAAGCGACGATCTGCTCGAGGGGATCTACCGCCAAAGCCGCGCGT |
| | | CGGCCCTAGGCCGCCGGTACATCGAGGCGAACCCAACAGCGCTGGCAAACCTGCTGGTC |
| | | GTGGACGTAGACCATCCAGACGCAGCGCTCCGAGCGCTCAGCGCCCGGGGGTCCCATCC |
| | | GCTGCCCAACGCGATCGTGGGCAATCGCGCCAACGGCCACGCACACGCAGTGTGGGCAC |
| | | TCAACGCCCCTGTTCCACGCACCGAATACGCGCGGCGTAAGCCGCTCGCATACATGGCG |
| | | GCGTGCGCCGAAGGCCTTCGGCGCGCCGTCGATGGCGACCGCAGTTACTCAGGCCTCAT |
| | | GACCAAAAACCCCGGCCACATCGCCTGGGAAACGGAATGGCTCCACTCAGATCTCTACAC |
| | | ACTCAGCCACATCGAGGCCGAGCTCGGCGCGAACATGCCACCGCCGCGCTGGCGTCAGC |
| | | AGACCACGTACAAAGCGGCTCCGACGCCGCTAGGGCGGAATTGCGCACTGTTCGATTCC |
| | | GTCAGGTTGTGGGCCTATCGTCCCGCCCTCATGCGGATCTACCTGCCGACCCGGAACGT |
| | | GGACGGACTCGGCCGCGCGATCTATGCCGAGTGCCACGCGCGAAACGCCGAATTTCCGT |
| | | GCAACGACGTGTGTCCCGGACCGCTACCGGACAGCGAGGTCCGCGCCATCGCCAACAGC |
| | | ATTTGGCGTTGGATCACAACCAAGTCGCGCATTTGGGCGGACGGGATCGTGGTCTACGAG |
| | | GCCACACTCAGTGCGCGCCAGTCGGCCATCTCGCGGAAGGGCGCAGCAGCGCGCACGG |
| | | CGGCGAGCACAGTTGCGCGGCGCGCAAAGTCCGCGTCAGCCATGGAGGCATTGCTATGA |
| | | GCGACGGCTACAGCGACGGCTACAACCGGCAGCCGACTGTCCGCAAAAAGCGGCGCGTG |
| | | ACCGCCGCCGAAGGCGCTCGAATCACCGGACTATCCGAACGCCACGTCGTCCGGCTCGT |
| | | GGCGCAGGAACGCAGCGAGTGGCTCGCCGAGCAGGCTGCACGCCGCGAACGCATCCGC |
| | | GCCTATCACGACGACGAGGGCCACTCTTGGCCGCAAACGGCCAAACATTTCGGGCTGCAT |
| | | CTGGACACCGTTAAGCGACTCGGCTATCGGGCGAGGAAAGAGCGTGCGGCAGAACAGGA |
| | | AGCGGCTCAAAAGGCCCACAACGAAGCCGACAATCCACCGCTGTTCTAACGCAATTGGGG |
| | | AGCGGGTGTCGCGGGGGTTCCGTGGGGGGTTCCGTTGCAACGGGTCGGACAGGTAAAA |
| | | GTCCTGGTAGACGCTAGTTTTCTGGTTTGGGCCATGCCTGTCTCGTTGCGTGTTTCGTTGC |
| | | GCCCGTTTTGAATACCAGCCAGACGAGACGGGGTTCTACGAATCTTGGTCGATACCAAGC |
| | | CATTTCCGCTGAATATCGGGGAGCTCACCGCCAGAATCGGTGGTTGTGGTGATGTACGTG |
| | | GCGAACTCCGTTGTAGTGCCTGTGGTGGCATCCGTGGCGCGGCCGCGGTACCAGATCTT |
| | | TAAATCTAGACGGTGACCACAACGCGCCCGCTTTGATCGGGGACGTCTGCGGCCGACCAT |
| | | TTACGGGTCTTGTTGTCGTTGGCGGTCATGGGCCGAACATACTCACCOGGATCGGAGGGC |
| | | CGAGGACAAGGTCGAACGAGGGGCATGACCCGGTGCGGGGCTTCTTGCACTCGGCATAG |
| | | GCGAGTGCTAAGAATAACGTTGGCACTCGCGACCGGTGAGTGCTAGGTCGGGACGGTGA |
| | | GGCCAGGCCCGTCGTCGCAGCGAGTGGCAGCGAGGACAACTTGAGCCGTCCGTCGCGG |
| | | GCACTGCGCCCGGCCAGCGTAAGTAGCGGGGTTGCCGTCACCCGGTGACCCCCGTTTCA |
| | | TCCCCGATCCGGAGGAATCACTTCGATATCGCCAAGACAATTGCGGATCATGACAATGCG |
| | | AATAGATACAGACAAACAAATGAATTTACTTAGTGATAAGAACGTTGCAATAATTGGTGGTG |
| | | GACCCGTTGGACTGACTATGGCAAAATTATTACAGCAAAACGGCATAGACGTTTCAGTTTA |
| | | CGAAAGAGACAACGACCGAGAGGCAAGAATTTTTGGTGGAACCCTTGACCTACACAAAGG |
| | | TTCAGGTCAGGAAGCAATGAAAAAAGCGGGATTGTTACAAACTTATTATGACTTAGCCTTAC |
| | | CAATGGGTGTAAATATTGCTGATAAAAAAGGCAATATTTTATCCACAAAAAATGTAAAGCCC |
| | | GAAAATCGATTTGACAATCCTGAAATAAACAGAAATGACTTAAGGGCTATCTTGTTGAATAG |
| | | TTTAGAAAACGACACGGTTATTTGGGATAGAAAACTTGTTATGCTTGAACCTGGTAAGAAGA |
| | | AGTGGACACTAACTTTTGAGAATAAACCGAGTGAAACAGCAGATTTGGTTATTCTTGCCAAT |
| | | GGCGGGATGTCCAAGGTAAGAAAATTTGTTACCGACACGGAAGTTGAAGAAACAGGTACT |
| | | TTCAATATACAAGCCGATATTCATCAACCAGAGATAAACTGTCCTGGATTTTTTCAGCTATG |
| | | CAATGGAAACCGGCTAATGGCATCTCACCAAGGTAATTTATTATTTGCTAACCCCAATAATA |
| | | ATGGTGCATTGCATTTTGGAATAAGTTTTAAAACACCTGATGAATGGAAAAACCAAACGCAG |
| | | GTAGATTTTCAAAACAGAAATAGTGTCGTTGATTTTCTTCTGAAAGAATTTTCCGATTGGGA |
| | | CGAACGCTACAAAGAATTGATTCATACGACGTTGTCATTTGTAGGATTGGCTACACGGATA |
| | | TTTCCTTTAGAAAAGCCTTGGAAAAGCAAGCGCCCATTACCCATAACAATGATTGGGGATG |
| | | CCGCACATTTGATGCCGCCTTTTGCAGGGCAGGGAGTAAATAGTGGGTTGGTGGATGCCT |
| | | TGATATTGTCTGATAATCTAGCCGATGGAAAATTTAATAGCATTGAAGAGGCTGTTAAAAAT |
| | | TATGAACAGCAAATGTTTATCTATGGCAAAGAAGCACAAGAAGAATCAACTCAAAACGAAAT |
| | | TGAAATGTTTAAACCCGACTTTACGTTTCAGCAATTGTTAAATGTATACCCATACGATGTTC |
| | | CAGATTACGCTTAAAGCTTATCGATGTCGACGTAGTTAACTAGCGTACGATCGACTGCCAG |
| | | GCATCAAATAAAACGAAAGGCTCAGTCGAAAGACTGGGCCTTTCGTTTTATGCCATCATGG |
| | | CCGCGGTGATCAGCTAGCCACCTGACGTCGGGGGGGGGGAAAGCCACGTTGTGTCTCAA |
| | | AATCTCTGATGTTACATTGCACAAGATAAAAATATATCATCATGAACAATAAAACTGTCTGCT |
| | | TACATAAACAGTAATACAAGGGGTGTTATGAGCCATATTCAACGGGAAACGTCTTGCTCGA |
| | | GGCCGCGATTAAATTCCAACATGGATGCTGATTTATATGGGTATAAATGGGCTCGCGATAA |
| | | TGTCGGGCAATCAGGTGCGACAATCTATCGCTTGTATGGGAAGCCCCATGCGCCAGAGTT |
| | | GTTTCTGAAACATGGCAAAGGTAGCGTTGCCAATGATGTTACAGATGAGATGGTCAGACTA |
| | | AACTGGCTGACGGAATTTATGCCTCTTCCGACCATCAAGCATTTTATCCGTACTCCTGATG |
| | | ATGCATGGTTACTCACCACTGCGATCCCCGGGAAAACAGCATTCCAGGTATTAGAAGAATA |
| | | TCCTGATTCAGGTGAAAATATTGTTGATGCGCTGGCAGTGTTCCTGCGCCGGTTGCATTCG |
| | | ATTCCTGTTTGTAATTGTCCTTTTAACAGCGATCGCGTATTTCGTCTCGCTCAGGCGCAATC |
| | | ACGAATGAATAACGGTTTGGTTGATGCGAGTGATTTTGATGACGAGCGTAATGGCTGGCCT |
| | | GTTGAACAAGTCTGGAAAGAAATGCATAATCTTTTGCCATTCTCACCGGATTCAGTCGTCA |
| | | CTCATGGTGATTTCTCACTTGATAACCTTATTTTTGACGAGGGGAAATTAATAGGTTGTATT |
| | | GATGTTGGACGAGTCGGAATCGCAGACCGATACCAGGATCTTGCCATCCTATGGAACTGC |
| | | CTCGGTGAGTTTTCTCCTTCATTACAGAAACGGCTTTTTCAAAAATATGGTATTGATAATCC |
| | | TGATATGAATAAATTGCAGTTTCATTTGATGCTCGATGAGTTTTTCTAATCAGAATTGGTTAA |
| | | TTGGTTGTAACACTGGCAGAGCATTACGCTGACTTGACGGGACGGCGGCTTTGTTGAATA |
| | | AATCGAACTTTTGCTGAGTTGAAGGATCAGATCACGCATCTTCCCGACAACGCAGACCGTT |
| | | CCGTGGCAAAGCAAAAGTTCAAAATCACCAACTGGTCCACCTACAACAAAGCTCTCACCAA |
| | | CCGTGGCTCCCTCACTTTCTGGCTGGATGATGGGGCGATTCAGGCCTGGTATGAGTCAGC |
| | | AACACCTTCTTCACGAGGCAGACCTCACTAGTTCCATGAGCGTCAGACCCCGTAGAAAAG |
| | | ATCAAAGGATCTTCTTGAGATCCTTTTTTTCTGCGCGTAATCTGCTGCTTGCAAAC |
| SEQ | pZE24 | CCAACCTTACCAGAGGGCGCCCCAGCTGGCAATTCCGACGTCTGTGTGGAATTGTGAGCG |
| ID | empty | GATAACAAATTCACACAGGGCCCTCGGACACCGAGGAGAATGTCAAGAGGCGAACACACA |
| NO: 3 | vector | ACGTCTTGGAGCGCCAGAGGAGGAACGAGCTAAAACGGAGCTTTTTTGCCCTGCGTGACC |
| | | AGATCCCGGAGTTGGAAAACAATGAAAAGGCCCCCAAGGTAGTTATCCTTAAAAAAGCCAC |
| | | AGCATACATCCTGTCCGTCCAAGCAGAGGAGCAAAAGCTCATTTCTGAAGAGGACTTGTTG |
| | | CGGAAACGACGAGAACAGTTGAAACACAAACTTGAACAGCTACGGAACTCTTGTGCGTAA |
| | | GGAAAAGTAAGGAAAACGATTCCTTCTAACAGAAATGTCCTGAGCAATCACCTATGAACTG |
| | | TCGACTCGAGCATAGCATTTTTATCCATAAGATTAGOGGATCCTAAGCTTTACAATTGTGAG |
| | | CGCTCACAATTATGATAGATTCAATTGTGAGCGGATAACAATTTCACACAGAATTCATTAAA |
| | | GAGGAGAAAGGTACCGGGCCCCCCCTCGAGGTCGACGGTATCGATAAGCTTGATATCGAA |
| | | TTCCTGCAGCCCGGGGGATCCCATGGTACGCGTGCTAGAGGCATCAAATAAAACGAAAGG |
| | | CTCAGTCGAAAGACTGGGCCTTTCGTTTTATCTGTTGTTTGTCGGTGAACGCTCTCCTGAG |
| | | TAGGACAAATCCGCCGCCCTAGACCTAGGGCGTTCGGCTGCGGCGAGCGGTATCAGCTC |
| | | ACTCAAAGGCGGTAATACGGTTATCCACAGAATCAGGGGATAACGCAGGAAAGAACATGT |
| | | GAGCAAAAGGCCAGCAAAAGGCCAGGAACCGTAAAAAGGCCGCGTTGCTGGCGTTTTTCC |
| | | ATAGGCTCCGCCCCCCTGACGAGCATCACAAAAATCGACGCTCAAGTCAGAGGTGGCGAA |
| | | ACCCGACAGGACTATAAAGATACCAGGCGTTTCCCCCTGGAAGCTCCCTCGTGCGCTCTC |
| | | CTGTTCCGACCCTGCCGCTTACCGGATACCTGTCCGCCTTTCTCCCTTCGGGAAGCGTGG |
| | | CGCTTTCTCATAGCTCACGCTGTAGGTATCTCAGTTCGGTGTAGGTCGTTCGCTCCAAGCT |
| | | GGGCTGTGTGCACGAACCCCCCGTTCAGCCCGACCGCTGCGCCTTATCCGGTAACTATCG |
| | | TCTTGAGTCCAACCCGGTAAGACACGACTTATCGCCACTGGCAGCAGCCACTGGTAACAG |
| | | GATTAGCAGAGCGAGGTATGTAGGCGGTGCTACAGAGTTCTTGAAGTGGTGGCCTAACTA |
| | | CGGCTACACTAGAAGGACAGTATTTGGTATCTGCGCTCTGCTGAAGCCAGTTACCTTCGGA |
| | | AAAAGAGTTGGTAGCTCTTGATCCGGCAAACAAACCACCGCTGGTAGCGGTGGTTTTTTTG |
| | | TTTGCAAGCAGCAGATTACGCGCAGAAAAAAAGGATCTCAAGAAGATCCTTTGATCTTTTCT |
| | | ACGGGGTCTGACGCTCAGTGGAACGAAAACTCACGTTAAGGGATTTTGGTCATGACTAGT |
| | | GCTTGGATTCTCACCAATAAAAAACGCCCGGCGGCAACCGAGCGTTCTGAACAAATCCAG |
| | | ATGGAGTTCTGAGGTCATTACTGGATCTATCAACAGGAGTCCAAGCGAGCTCTCGAACCC |
| | | CAGAGTCCCGCTCAGAAGAACTCGTCAAGAAGGCGATAGAAGGCGATGCGCTGCGAATC |
| | | GGGAGCGGCGATACCGTAAAGCACGAGGAAGCGGTCAGCCCATTCGCCGCCAAGCTCTT |
| | | CAGCAATATCACGGGTAGCCAACGCTATGTCCTGATAGCGGTCCGCCACACCCAGCCGGC |
| | | CACAGTCGATGAATCCAGAAAAGCGGCCATTTTCCACCATGATATTCGGCAAGCAGGCATC |
| | | GCCATGGGTCACGACGAGATCCTCGCCGTCGGGCATGCGCGCCTTGAGCCTGGCGAACA |
| | | GTTCGGCTGGCGCGAGCCCCTGATGCTCTTCGTCCAGATCATCCTGATCGACAAGACCGG |
| | | CTTCCATCCGAGTACGTGCTCGCTCGATGCGATGTTTCGCTTGGTGGTCGAATGGGCAGG |
| | | TAGCCGGATCAAGCGTATGCAGCCGCCGCATTGCATCAGCCATGATGGATACTTTCTCGG |
| | | CAGGAGCAAGGTGAGATGACAGGAGATCCTGCCCCGGCACTTCGCCCAATAGCAGCCAG |
| | | TCCCTTCCCGCTTCAGTGACAACGTCGAGCACAGCTGCGCAAGGAACGCCCGTCGTGGC |
| | | CAGCCACGATAGCCGCGCTGCCTCGTCCTGCAGTTCATTCAGGGCACCGGACAGGTCGG |
| | | TCTTGACAAAAAGAACCGGGCGCCCCTGCGCTGACAGCCGGAACACGGCGGCATCAGAG |
| | | CAGCCGATTGTCTGTTGTGCCCAGTCATAGCCGAATAGCCTCTCCACCCAAGCGGCCGGA |
| | | GAACCTGCGTGCAATCCATCTTGTTCAATCATGCGAAACGATCCTCATCCTGTCTCTTGAT |
| | | CAGATCTTGATCCCCTGCGCCATCAGATCCTTGGCGGCAAGAAAGCCATCCAGTTTACTTT |
| | | GCAGGGCTTC |
| SEQ | pZE24- | GGATCCCATGGTACGCGTGCTAGAGGCATCAAATAAAACGAAAGGCTCAGTCGAAAGACT |
| ID | Tet(X7) | GGGCCTTTCGTTTTATCTGTTGTTTGTCGGTGAACGCTCTCCTGAGTAGGACAAATCCGCC |
| NO: 4 | | GCCCTAGACCTAGGGCGTTCGGCTGCGGCGAGCGGTATCAGCTCACTCAAAGGCGGTAA |
| | | TACGGTTATCCACAGAATCAGGGGATAACGCAGGAAAGAACATGTGAGCAAAAGGCCAGC |
| | | AAAAGGCCAGGAACCGTAAAAAGGCCGCGTTGCTGGCGTTTTTCCATAGGCTCCGCCCCC |
| | | CTGACGAGCATCACAAAAATCGACGCTCAAGTCAGAGGTGGCGAAACCCGACAGGACTAT |
| | | AAAGATACCAGGCGTTTCCCCCTGGAAGCTCCCTCGTGCGCTCTCCTGTTCCGACCCTGC |
| | | CGCTTACCGGATACCTGTCCGCCTTTCTCCCTTCGGGAAGCGTGGCGCTTTCTCATAGCT |
| | | CACGCTGTAGGTATCTCAGTTCGGTGTAGGTCGTTCGCTCCAAGCTGGGCTGTGTGCACG |
| | | AACCCCCCGTTCAGCCCGACCGCTGCGCCTTATCCGGTAACTATCGTCTTGAGTCCAACC |
| | | CGGTAAGACACGACTTATCGCCACTGGCAGCAGCCACTGGTAACAGGATTAGCAGAGCGA |
| | | GGTATGTAGGCGGTGCTACAGAGTTCTTGAAGTGGTGGCCTAACTACGGCTACACTAGAA |
| | | GGACAGTATTTGGTATCTGCGCTCTGCTGAAGCCAGTTACCTTCGGAAAAAGAGTTGGTAG |
| | | CTCTTGATCCGGCAAACAAACCACCGCTGGTAGCGGTGGTTTTTTTGTTTGCAAGCAGCAG |
| | | ATTACGCGCAGAAAAAAAGGATCTCAAGAAGATCCTTTGATCTTTTCTACGGGGTCTGACG |
| | | CTCAGTGGAACGAAAACTCACGTTAAGGGATTTTGGTCATGACTAGTGCTTGGATTCTCAC |
| | | CAATAAAAAACGCCCGGCGGCAACCGAGCGTTCTGAACAAATCCAGATGGAGTTCTGAGG |
| | | TCATTACTGGATCTATCAACAGGAGTCCAAGCGAGCTCTCGAACCCCAGAGTCCCGCTCA |
| | | GAAGAACTCGTCAAGAAGGCGATAGAAGGCGATGCGCTGCGAATCGGGAGCGGCGATAC |
| | | CGTAAAGCACGAGGAAGCGGTCAGCCCATTCGCCGCCAAGCTCTTCAGCAATATCACGGG |
| | | TAGCCAACGCTATGTCCTGATAGCGGTCCGCCACACCCAGCCGGCCACAGTCGATGAATC |
| | | CAGAAAAGCGGCCATTTTCCACCATGATATTCGGCAAGCAGGCATCGCCATGGGTCACGA |
| | | CGAGATCCTCGCCGTCGGGCATGCGCGCCTTGAGCCTGGCGAACAGTTCGGCTGGCGCG |
| | | AGCCCCTGATGCTCTTCGTCCAGATCATCCTGATCGACAAGACCGGCTTCCATCCGAGTA |
| | | CGTGCTCGCTCGATGCGATGTTTCGCTTGGTGGTCGAATGGGCAGGTAGCCGGATCAAGC |
| | | GTATGCAGCCGCCGCATTGCATCAGCCATGATGGATACTTTCTCGGCAGGAGCAAGGTGA |
| | | GATGACAGGAGATCCTGCCCCGGCACTTCGCCCAATAGCAGCCAGTCCCTTCCCGCTTCA |
| | | GTGACAACGTCGAGCACAGCTGCGCAAGGAACGCCCGTCGTGGCCAGCCACGATAGCCG |
| | | CGCTGCCTCGTCCTGCAGTTCATTCAGGGCACCGGACAGGTCGGTCTTGACAAAAAGAAC |
| | | CGGGCGCCCCTGCGCTGACAGCCGGAACACGGCGGCATCAGAGCAGCCGATTGTCTGTT |
| | | GTGCCCAGTCATAGCCGAATAGCCTCTCCACCCAAGCGGCCGGAGAACCTGCGTGCAATC |
| | | CATCTTGTTCAATCATGCGAAACGATCCTCATCCTGTCTCTTGATCAGATCTTGATCCCCTG |
| | | CGCCATCAGATCCTTGGCGGCAAGAAAGCCATCCAGTTTACTTTGCAGGGCTTCCCAACC |
| | | TTACCAGAGGGCGCCCCAGCTGGCAATTCCGACGTCTGTGTGGAATTGTGAGCGGATAAC |
| | | AAATTCACACAGGGCCCTCGGACACCGAGGAGAATGTCAAGAGGCGAACACACAACGTCT |
| | | TGGAGCGCCAGAGGAGGAACGAGCTAAAACGGAGCTTTTTTGCCCTGCGTGACCAGATCC |
| | | CGGAGTTGGAAAACAATGAAAAGGCCCCCAAGGTAGTTATCCTTAAAAAAGCCACAGCATA |
| | | CATCCTGTCCGTCCAAGCAGAGGAGCAAAAGCTCATTTCTGAAGAGGACTTGTTGCGGAA |
| | | ACGACGAGAACAGTTGAAACACAAACTTGAACAGCTACGGAACTCTTGTGCGTAAGGAAAA |
| | | GTAAGGAAAACGATTCCTTCTAACAGAAATGTCCTGAGCAATCACCTATGAACTGTCGACT |
| | | CGAGCATAGCATTTTTATCCATAAGATTAGCGGATCCTAAGCTTTACAATTGTGAGCGCTCA |
| | | CAATTATGATAGATTCAATTGTGAGCGGATAACAATTTCACACAGAATTCATTAAAGAGGAG |
| | | AAAGGTACCATGACTTTGCTAAAAAAAAAAAAATTACAATAATTGGTGCCGGGCCTGTTGG |
| | | ATTAACGATGGCGAGATTGTTACAGCAAAACCGCGTGGACGTTACAGTTTACGAGAGAGA |
| | | CAAAGACCGAGATGCAAGGATTTTTGGTGGGACACTTGACCTGCACAGGGATTCGGGACA |
| | | GGAAGCAATGAAAAGAGCGGGATTGTTACAAACTTATTATGACTTAGCTTTACCAATGGGT |
| | | GTAAATATTGCTGATGAAAAGGGTAATATTTTAACCACAAAAAATGTAAAGCCCGAAAATCG |
| | | GTTTGACAATCCTGAAATAAACAGAAATGACTTAAGAACTATCTTATTAAATAGCCTACAAAA |
| | | TGACACCGTCATTTGGGATAGAAAACTTGTTACGCTTGAACCTGATAAGGAGAAGTGGACA |
| | | CTAACTTTTGAAGATAAACCGAGTGAAACAGCAGATCTGGTTATTATTGCCAATGGTGGAAT |
| | | GTCTAAAGTAAGAAAATTTGTTACCGACACGGAAGTTGAAGAAACAGGTACTTTCAATATAC |
| | | AAGCCGATATTCATCAACCAGAGGTGAACTGTCCTGGATTTTTTCAGCTATGCAATGGAAA |
| | | CCGGCTAATGGCTGCTCATCAAGGTAATTTATTATTTGCGAATCCTAATAATAATGGTGCAT |
| | | TGCATTTTGGAATAAGTTTTAAAACATCTGATGAATGGAAAAGCAAGACTCTGGTAGATTTT |
| | | CAAGACAGAAATAGTGTCGTTGATTTTCTCCTGAAAAAATTTTCCGATTGGGATGAACGCTA |
| | | CAAAGAACTGATTCGTGTGACATCATCTTTTGTAGGGTTAGCGACACGAATATTTCCCTTAG |
| | | GTAAGTCTTGGAAAAGTAAGCGTCCATTACCCATAACGATGATTGGAGATGCTGCTCATTT |
| | | GATGCCTCCTTTTGCAGGACAAGGCGTAAACAGCGGGTTGATGGATGCCTTGATATTGTC |
| | | GGATAATCTGACCAATGGGAAATTTAACAGCATTGAAGAGGCTATTGAAAATTATGAACAG |
| | | CAAATGTTTGCTTATGGAAGAGAAGCACAGGCAGAATCAATAATAAACGAAACGGAAATGT |
| | | TCAGTTCCGACTTTTCATTCCAAAAACTAATGAATCTATAA |
| SEQ | pZE24- | GGATCCCATGGTACGCGTGCTAGAGGCATCAAATAAAACGAAAGGCTCAGTCGAAAGACT |
| ID | Tet(50) | GGGCCTTTCGTTTTATCTGTTGTTTGTCGGTGAACGCTCTCCTGAGTAGGACAAATCCGCC |
| NO: 5 | | GCCCTAGACCTAGGGCGTTCGGCTGCGGCGAGCGGTATCAGCTCACTCAAAGGCGGTAA |
| | | TACGGTTATCCACAGAATCAGGGGATAACGCAGGAAAGAACATGTGAGCAAAAGGCCAGC |
| | | AAAAGGCCAGGAACCGTAAAAAGGCCGCGTTGCTGGCGTTTTTCCATAGGCTCCGCCCCC |
| | | CTGACGAGCATCACAAAAATCGACGCTCAAGTCAGAGGTGGCGAAACCCGACAGGACTAT |
| | | AAAGATACCAGGCGTTTCCCCCTGGAAGCTCCCTCGTGCGCTCTCCTGTTCCGACCCTGC |
| | | CGCTTACCGGATACCTGTCCGCCTTTCTCCCTTCGGGAAGCGTGGCGCTTTCTCATAGCT |
| | | CACGCTGTAGGTATCTCAGTTCGGTGTAGGTCGTTCGCTCCAAGCTGGGCTGTGTGCACG |
| | | AACCCCCCGTTCAGCCCGACCGCTGCGCCTTATCCGGTAACTATCGTCTTGAGTCCAACC |
| | | CGGTAAGACACGACTTATCGCCACTGGCAGCAGCCACTGGTAACAGGATTAGCAGAGCGA |
| | | GGTATGTAGGCGGTGCTACAGAGTTCTTGAAGTGGTGGCCTAACTACGGCTACACTAGAA |
| | | GGACAGTATTTGGTATCTGCGCTCTGCTGAAGCCAGTTACCTTCGGAAAAAGAGTTGGTAG |
| | | CTCTTGATCCGGCAAACAAACCACCGCTGGTAGCGGTGGTTTTTTTGTTTGCAAGCAGCAG |
| | | ATTACGCGCAGAAAAAAAGGATCTCAAGAAGATCCTTTGATCTTTTCTACGGGGTCTGACG |
| | | CTCAGTGGAACGAAAACTCACGTTAAGGGATTTTGGTCATGACTAGTGCTTGGATTCTCAC |
| | | CAATAAAAAACGCCCGGCGGCAACCGAGCGTTCTGAACAAATCCAGATGGAGTTCTGAGG |
| | | TCATTACTGGATCTATCAACAGGAGTCCAAGCGAGCTCTCGAACCCCAGAGTCCCGCTCA |
| | | GAAGAACTCGTCAAGAAGGCGATAGAAGGCGATGCGCTGCGAATCGGGAGCGGCGATAC |
| | | CGTAAAGCACGAGGAAGCGGTCAGCCCATTCGCCGCCAAGCTCTTCAGCAATATCACGGG |
| | | TAGCCAACGCTATGTCCTGATAGCGGTCCGCCACACCCAGCCGGCCACAGTCGATGAATC |
| | | CAGAAAAGCGGCCATTTTCCACCATGATATTCGGCAAGCAGGCATCGCCATGGGTCACGA |
| | | CGAGATCCTCGCCGTCGGGCATGCGCGCCTTGAGCCTGGCGAACAGTTCGGCTGGCGCG |
| | | AGCCCCTGATGCTCTTCGTCCAGATCATCCTGATCGACAAGACCGGCTTCCATCCGAGTA |
| | | CGTGCTCGCTCGATGCGATGTTTCGCTTGGTGGTCGAATGGGCAGGTAGCCGGATCAAGC |
| | | GTATGCAGCCGCCGCATTGCATCAGCCATGATGGATACTTTCTCGGCAGGAGCAAGGTGA |
| | | GATGACAGGAGATCCTGCCCCGGCACTTCGCCCAATAGCAGCCAGTCCCTTCCCGCTTCA |
| | | GTGACAACGTCGAGCACAGCTGCGCAAGGAACGCCCGTCGTGGCCAGCCACGATAGCCG |
| | | CGCTGCCTCGTCCTGCAGTTCATTCAGGGCACCGGACAGGTCGGTCTTGACAAAAAGAAC |
| | | CGGGCGCCCCTGCGCTGACAGCCGGAACACGGCGGCATCAGAGCAGCCGATTGTCTGTT |
| | | GTGCCCAGTCATAGCCGAATAGCCTCTCCACCCAAGCGGCCGGAGAACCTGCGTGCAATC |
| | | CATCTTGTTCAATCATGCGAAACGATCCTCATCCTGTCTCTTGATCAGATCTTGATCCCCTG |
| | | CGCCATCAGATCCTTGGCGGCAAGAAAGCCATCCAGTTTACTTTGCAGGGCTTCCCAACC |
| | | TTACCAGAGGGCGCCCCAGCTGGCAATTCCGACGTCTGTGTGGAATTGTGAGCGGATAAC |
| | | AAATTCACACAGGGCCCTCGGACACCGAGGAGAATGTCAAGAGGCGAACACACAACGTCT |
| | | TGGAGCGCCAGAGGAGGAACGAGCTAAAACGGAGCTTTTTTGCCCTGCGTGACCAGATCC |
| | | CGGAGTTGGAAAACAATGAAAAGGCCCCCAAGGTAGTTATCCTTAAAAAAGCCACAGCATA |
| | | CATCCTGTCCGTCCAAGCAGAGGAGCAAAAGCTCATTTCTGAAGAGGACTTGTTGCGGAA |
| | | ACGACGAGAACAGTTGAAACACAAACTTGAACAGCTACGGAACTCTTGTGCGTAAGGAAAA |
| | | GTAAGGAAAACGATTCCTTCTAACAGAAATGTCCTGAGCAATCACCTATGAACTGTCGACT |
| | | CGAGCATAGCATTTTTATCCATAAGATTAGCGGATCCTAAGCTTTACAATTGTGAGCGCTCA |
| | | CAATTATGATAGATTCAATTGTGAGCGGATAACAATTTCACACAGAATTCATTAAAGAGGAG |
| | | AAAGGTACCATGACTAAACATATAAAAATTCTTGTAATAGGGGTTGGGGTTGCGGGCCCTG |
| | | CAGTCGCTTATTGGTTAAAAAGATTTGGGTTTTCTCCGGTCTTAATTGAAAAATCAGCTGCC |
| | | GTTAGAAAAGGGGGCCAGGCACTTGATATTCGCGGCATAGCCACACATATCGCTAAAGAA |
| | | ATGGGCATTTACGACCAGATATGTAACATGCGGACTCAAATAAAGTGTGGACGTTATGTGG |
| | | ATGTAAAGGGTAATGTTTTGCATGAAGAACAAGGTGAAACCTTTGGTTTCCGGCAAGATGA |
| | | CGAAGTTGAGATTCTTCGTGGAGACCTAGTTGAAATTCTAATGAAAGCAATTGCAGATATAC |
| | | CTTGTGAATTCAAACAATCCGTCATAAAAATTGAACAAAACGAAGATAGTGTAACTGTCACC |
| | | TACAAAGATGGCAGAGTTGAAAATTATGACCTTGTGATCGCTGCCGATGGTATTCATTCTG |
| | | CTACCAGGGGAATGGTTTTTAGTAAAAATGAATATCAGTTGATTAATCTCGGCTCCTACGTC |
| | | AGCGCATTTACCATCCCAAATTATCTCGGTTTGGATCACATGGAACTCCTATGTGAATCAAA |
| | | TCATAAACTGGTTACTCTACAAAGCGACAGCCAAGCTGACAAAGCCATGGCGGGATTTATG |
| | | TTCCGTTCAAAACATGTATTAGAAGATATTCGTGATGAGCAAGAACAAAAACATTTCTTACA |
| | | CGCCAGTTTTCAAAATTTCGGTTGGGAAACACAAAATATTTTAAATCGAATGCCAGAAAGCG |
| | | ATGACTTTTATTTTGACGCGATTACTCAAATCAAAATGAAAAGCTGGACTAAAGGCCGTATA |
| | | GCCTTAATCGGCGATGCCGCTTATTGCCCCTCACCTTTATCAGGTCAGGGGAATAATTTAG |
| | | CTTTTGTTGGCGCTTATATTTTGGGGGTGAATTGAAAAAGGCTGATGGTGATTATATACAA |
| | | GCATTTACCCGATATAACGAATTGCTACATCCCTTTGTTGAGGCTAACCAACAATTTGGTGT |
| | | GTGGGTAAGCGAATCCTTTCTCCTAAAAGATGATGAAGTCTCTAAAGAGATTGCGGAAGCC |
| | | CGTTCTAATAAAATTTTGGCAATGATAAAGTCAGTCTCTAATTCAATAAACTTGCCACAATAC |
| | | GAATAA |

### General Materials and Methods

All in vitro kinetic assays and whole cell bacterial growth assays were prepared open to air in non-degassed buffer solutions using sterile technique. All organic solvents including deuterated NMR solvents and reagent chemicals used in preparation or analysis of synthetic compounds were obtained commercially and used without further purification. TC (HCl salt) and NADPH (tetrasodium salt) were purchased from Millipore-Sigma (St. Louis, MO). aTC (HCl salt) was purchased from Chemodex (United Kingdom). NMR spectra were obtained on a Varian Unity-Inova 500 MHz or Agilent Premium Compact+ 600 MHz spectrometer in 5 mm type 1, class A borosilicate glass NMR tubes (Wilman LabGlass part No. 535-PP-8). All free induction decay files (FIDs) were processed using Mestrenova version 11.0.4 software. Chemical shifts (δ) are reported in parts per million (ppm) and referenced to residual non-deuterated solvent. Coupling constants (J are reported in hertz (Hz). TDase in vitro reactions were monitored by optical absorbance spectroscopy on an Agilent Cary 50 UV-visible spectrophotometer using polystyrene cuvettes and LC-MS using an Agilent 6130 single quadrupole instrument (ESI+) with G1313 autosampler, G1315 diode array detector, and 1200 series solvent module with separation on a Phenomenex Gemini C18 column, 50×2 mm (5 μm) fit with a guard column cassette. LC-MS solvents were 0.1% formic acid in H2O (A) and 0.1% formic acid in ACN (B). Solvent gradient was linear starting from 0% B to 95% B over 20 min at a flow rate of 0.5 mL/min. HPLC was performed on an HP1050 system using a Luna 10 mm C18(2) 100 Å column (250 mm×21.2 mm) from Phenomenex fit with a guard column of the same matrix (15 mm×21.2 mm). HPLC solvents were 0.1% formic acid in H2O (A) and 0.1% formic acid in ACN (B) with a gradient formed from 0% B to 95% B over 20 min. LC-MS and HPLC data were processed using ChemStation software version B.04.02 SP1. Liquid medium bacterial growth assays were performed using Difco BBL Mueller-Hinton broth in Costar 96-well plates at 37° C. Endpoint growth density was judged by OD600 measurement using a Synergy H1 plate reader (BioTek, Inc.). SDS-PAGE analysis was carried out using Bio-Rad Any kD precast polyacrylamide gels with staining by Coomassie brilliant blue and comparison to a Bio-Rad precision plus protein dual Xtra pre-stained protein standard ladder. Human blood plasma stability experiments were performed using Innovative Research Pooled Human Plasma (Blood Derived).

### Hemolysis.

Human red blood cells (Innovative Research) were prepared by adding 2 mL of 25% RBCS to a 15 mL tube under aseptic technique. The RBCs were centrifuged at 1450×g for 10 min at 4° C. Supernatant was removed by pipette and gently resuspended in 12.5 mL of cold PBS, yielding a 4% (v/v) concentration of RBCs. A 96-well plate with the corresponding compound was prepared by adding 150 μL of 1× PBS to all sample wells except the first and last columns. 300 μL of the corresponding compound at 1 mM was added to column 1 in duplicate and diluted twofold down the plate by transferring 150 uL, leaving the last column blank. A second plate was prepared by adding 100 μL of the 4% (v/v) RBCs to all wells, leaving 2 empty as blank controls. 100 μL of each well from plate 1, containing the compounds, was transferred and gently mixed, careful not to cause lysing. In the last column, 100 μL of PBS was added to 3 wells containing blood to serve as a negative control, and 100 μL of PBS containing 1% (v/v) Triton X-100 was added to 3 wells containing blood to serve as a lysis positive control. The plate was incubated for 1 hour at 37° C. then gently mixed. The absorbance at 650 nm was measured. The positive control had average Abs650 of 0.06 and the negative control had an average Abs₆₅₀ of 1.3.

### Inhibitor Stability in Bacterial Cell Lysate.

E. coli MegaX pZE21 cells were cultured at 37° C. in lysogeny broth (LB) containing kanamycin (Kan) at 0.05 mg/mL (final concentration) and grown to an OD600 of ˜0.8. The cells were centrifuged (5000 rpm, RT) for 15 min, resuspended in 10 mL 50 mM TRIS (pH 8.0), and centrifuged again. The cells were resuspended in 5 mL 50 mM TRIS and lysed using sonication. The resultant lysate was clarified via centrifugation at 13,000 rpm for 15 min at 4° C. 50 μL of 10 mM compound in DMSO was added to 450 μL cell lysate. The treated lysate was incubated at 37° C. with gentle mixing and at each time point (0 min, 30 min, 18 hours) in duplicate, 100 μL reaction mixture was removed and added to 500 μL of quench solution (50% acetonitrile, 50% 0.25 M HCl). The quenched samples were centrifuged (5000 rpm, RT) for 10 min, and 500 μL of the resulting supernatant was mixed with 20 μL Fmoc-Ala (250 μM in acetonitrile) and analyzed by LC-MS in positive ion mode.

### Inhibitor Stability in Human Blood Plasma.

Human blood plasma (Innovative Research) was diluted to 50% in TRIS (pH 7.5). 50 μL of 10 mM compound in DMSO was added to 450 μL plasma. The treated plasma was incubated at 37° C. with gentle mixing and at each time point (0 min, 30 min, 18 hours) in duplicate, 100 μL reaction mixture was removed and added to 500 μL of quench solution (50% acetonitrile, 50% 0.25 M HCl). The quenched samples were centrifuged (5000 rpm, room temperature) for 10 min, and 500 μL of the resulting supernatant was mixed with 2 μL Fmoc-Ala (250 μM in acetonitrile) and analyzed by LC-MS in positive ion mode.

### Example 2—Synthesis of C9-Bezamide and C10-Benzoate Ester Atc Derivatives

### General Procedure (A) for the Synthesis of C10-Benzoate aTC Analogues (2-15)

The corresponding benzoic acid (0.0199 g, 0.162 mmol, 1.00 equiv) and DIPEA (0.045 mL, 0.260 mmol, 1.60 equiv) were dissolved in 3.5 mL of anhydrous DMF (DriSolv) in a vial and charged with HATU (0.0617 g, 0.162 mmol, 1.00 equiv). The clear solution turned pale yellow and was stirred at room temperature for 10 min before the addition of compound aTC (HCl salt). The reaction solution was stirred for 5 min at room temperature and concentrated under reduced pressure via rotary evaporation. The residue was dissolved in MeOH, filtered through a 0.45 μm PTFE syringe filter, and purified by RPC18 prep-HPLC to provide the desired products 2-15 as the corresponding formic acid salts. All compounds used in biological assays are >95% pure as judged by analytical LCMS. Epimerization of some compounds, presumably at C4, was observed. The ‘epimeric purity’ of each test compound was determined by LCMS and is reported.

### C10-Benzoate Ester aTC Analogue 2: (6aS,7S,10aS)-9-Carbamoyl-7-(dimethylamino)-8,10a,12-trihydroxy-5-methyl-10,11-dioxo-6,6a,7,10,10a,11-hexahydrotetracen-1-yl benzoate

Compound 2 was prepared according to general procedure A and was obtained in 25% yield as a yellow-green solid. 1H NMR (600 MHz, DMSO-d6) δ 8.18 (d, J=7.8 Hz, 2H), 8.06 (d, J=8.5 Hz, 1H), 7.84 (t, J=7.8 Hz, 1H), 7.78 (t, J=7.2 Hz, 1H), 7.64 (t, J=7.6 Hz, 2H), 7.40 (d, J=7.3 Hz, 1H), 2.46 (s, 1H). 13C NMR (151 MHz, DMSO-d6) δ 172.5, 165.1, 160.6, 149.1, 149.1, 138.9, 138.8, 133.8, 132.9, 131.7, 131.4, 129.9, 129.5, 129.2, 128.9, 128.6, 122.8, 122.2, 121.8, 120.2, 116.7, 116.5, 110.3, 76.5, 66.2, 37.9, 26.2, 14.2, 14.2. MS (ESI+): [M+H]+; found, 530.7 (error <0.0005 Da). LCMS epimeric purity 95%. LCMS tr=12.7 min.

### C10-Benzoate Ester aTC Analogue 3: (6aS,7S,10aS)-9-Carbamoyl-7-(dimethylamino)-8,10a,12-trihydroxy-5-methyl-10,11-dioxo-6,6a,7,10,10a,11-hexahydrotetracen-1-yl 2-fluorobenzoate

Compound 3 was prepared according to general procedure A and was obtained in 76% yield as a green oil. 1H NMR (600 MHz, DMSO-d6) δ 8.15 (dd, J=6.3, 14.0 Hz, 1H), 8.06 (d, J=8.6 Hz, 1H), 7.80 (dt, J=7.5, 21.3 Hz, 2H), 7.44 (q, J=7.5 Hz, 2H), 7.38 (d, J=7.4 Hz, 1H), 3.39 (d, J=17.1 Hz, 2H), 3.24-3.18 (m, 1H), 2.75 (s, 1H), 2.51 (s, 3H), 2.41 (s, 6H). 13C NMR (151 MHz, DMSO-d6) δ 199.9, 172.8, 163.0, 162.3, 161.7, 160.6, 160.2, 148.6, 139.1, 138.6, 135.8, 135.8, 132.8, 132.4, 131.1, 124.9, 124.8, 124.8, 122.9, 122.1, 121.8, 119.9, 117.9, 117.8, 117.3, 117.3, 117.1, 116.4, 110.5, 110.2, 76.7, 41.7, 29.0, 26.7, 14.2. MS (ESI+): [M+H]+; found, 548.7. HRMS (ESI+): calcd [M+H]+, 549.1668; found 549.1675 (error <0.0005 Da). LCMS epimeric purity 99%. LCMS tr=12.0 min.

### C10-Benzoate Ester aTC Analogue 4: (6aS,7S,10aS)-9-Carbamoyl-7-(dimethylamino)-8,10a,12-trihydroxy-5-methyl-10,11-dioxo-6,6a,7,10,10a,11-hexahydrotetracen-1-yl 3-fluorobenzoate (4)

Compound 4 was prepared according to general procedure A and was obtained in 63% yield as a green oil. 1H NMR (300 MHz, DMSO-d6) δ 8.09-7.61 (m, 6H), 7.39 (d, J=7.5 Hz, 1H), 4.55 (s, 1H), 3.49-3.15 (m, 2H), 2.84 (s, 1H), 2.45 (s, 1H), 2.41 (s, 2H). MS (ESI+): [M+H]+; found, 548.7. HRMS (ESI+): calcd [M+H]+, 549.1668; found 549.1691 (error <0.0005 Da). LCMS epimeric purity 35%. LCMS tr=12.7 min.

### C10-Benzoate Ester aTC 5: (6aS,7S,10aS)-9-Carbamoyl-7-(dimethylamino)-8,10a,12-trihydroxy-5-methyl-10,11-dioxo-6,6a,7,10,10a,11-hexahydrotetracen-1-yl 4-fluorobenzoate (5)

Compound 5 was prepared according to general procedure A and was obtained in 21% yield as a green oil. 1H NMR (600 MHz, DMSO-6) δ 8.23-8.18 (m, 3H), 8.05 (d, J=8.6 Hz, 1H), 7.81 (t, J=8.1 Hz, 1H), 7.44 (t, J=8.6 Hz, 2H), 7.36 (d, J=7.4 Hz, 1H), 3.23-3.17 (m, 2H), 2.74 (s, 2H), 2.45 (s, 1H), 2.40 (s, 6H). 13C NMR (151 MHz, DMSO-d6) δ 199.9, 172.7, 166.2, 164.5, 164.2, 163.0, 161.8, 160.3, 148.9, 148.9, 139.3, 139.1, 138.6, 132.9, 132.8, 132.7, 132.6, 131.1, 126.2, 122.8, 122.7, 122.1, 121.8, 119.9, 116.5, 116.2, 116.1, 115.9, 110.5, 110.2, 76.7, 41.7, 31.1, 29.0, 26.7, 22.6, 22.0, 14.2, 14.2. MS (ESI+): [M+H]+; found, 549.0. HRMS (ESI+): calcd [M+H]+, 549.1667; found 549.1685 (error <0.0005 Da). LCMS epimeric purity 98%. LCMS tr=12.7 min.

### C10-Benzoate Ester aTC Analogue 6: (6aS,7S,10aS)-9-Carbamoyl-7-(dimethylamino)-8,10a,12-trihydroxy-5-methyl-10,11-dioxo-6,6a,7,10,10a,11-hexahydrotetracen-1-yl 4-(trifluoromethyl)benzoate

Compound 6 was prepared according to general procedure A and was obtained in 82% yield as a green oil. 1H NMR (600 MHz, DMSO-d6) δ 8.35 (d, J=8.1 Hz, 2H), 8.07 (d, J=8.7 Hz, 1H), 8.00 (d, J=8.2 Hz, 2H), 7.83 (t, J=8.1 Hz, 1H), 7.46 (t, J=7.0 Hz, 1H), 7.41 (d, J=7.5 Hz, 1H), 4.44 (d, J=5.8 Hz, 1H), 3.38 (d, J=16.7 Hz, 1H), 3.24-3.18 (m, 1H), 2.77-2.72 (m, 2H), 2.52 (s, 3H), 2.41 (s, 5H). 13C NMR (151 MHz, DMSO-d6) δ 164.1, 161.6, 148.7, 139.1, 133.3, 133.0, 132.8, 131.1, 130.7, 125.9, 122.9, 122.1, 119.9, 116.3, 110.2, 40.4, 40.4, 14.2. MS (ESI+): [M+H]+; found, 598.8. HRMS (ESI+): calcd [M+H]+, 599.1636; found 599.1631 (error <0.0005 Da). LCMS epimeric purity 98%. LCMS tr=12.7 min.

### C10-Benzoate Ester aTC Analogue 7: (6aS,7S,10aS)-9-Carbamoyl-7-(dimethylamino)-8,10a,12-trihydroxy-5-methyl-10,11-dioxo-6,6a,7,10,10a,11-hexahydrotetracen-1-yl picolinate

Compound 7 was prepared according to general procedure A and was obtained in 90% yield as a yellow oil. 1H NMR (600 MHz, DMSO-d6) δ 8.84 (dd, J=4.2, 19.1 Hz, 1H), 8.32 (d, J=7.7 Hz, 1H), 8.26 (d, J=7.8 Hz, 1H), 8.15-8.02 (m, 3H), 7.85-7.79 (m, 1H), 7.76 (ddd, J=5.1, 7.2, 17.8 Hz, 2H), 7.39 (d, J=7.5 Hz, 1H), 3.44 (d, J=5.3 Hz, 1H), 3.39 (d, J=16.5 Hz, 2H), 3.24-3.17 (m, 2H), 2.99 (dd, J=12.0, 17.5 Hz, 1H), 2.51 (s, 2H), 2.46 (s, 1H), 2.41 (s, 4H). 13C NMR (151 MHz, DMSO-d6) δ 199.9, 172.8, 163.8, 163.0, 161.7, 150.0, 149.0, 147.2, 139.1, 137.7, 132.7, 131.2, 127.7, 125.7, 122.8, 122.0, 119.7, 116.4, 110.2, 76.7, 41.7, 26.6, 24.8, 23.4, 20.7, 19.2, 14.2. MS (ESI+): [M+H]+; found, 532.3. HRMS (ESI+): calcd [M+H]+, 533.1714; found 533.1721 (error <0.0005 Da). LCMS epimeric purity 99%. LCMS tr=12.7 min.

### C10-Benzoate Ester aTC Analogue 8: (6aS,7S,10aS)-9-Carbamoyl-7-(dimethylamino)-8,10a,12-trihydroxy-5-methyl-10,11-dioxo-6,6a,7,10,10a,11-hexahydrotetracen-1-yl nicotinate

Compound 8 was prepared according to general procedure A and was obtained in 88% yield as a yellow oil. 1H NMR (600 MHz, DMSO-d6) δ 9.32 (d, J=1.7 Hz, 1H), 9.28 (d, J=1.6 Hz, 1H), 8.97-8.89 (m, 2H), 8.53 (d, J=7.9 Hz, 1H), 8.48 (d, J=7.9 Hz, 1H), 8.06 (t, J=9.2 Hz, 1H), 7.82 (td, J=3.0, 8.2 Hz, 1H), 7.68 (ddd, J=4.9, 7.8, 20.4 Hz, 1H), 7.42 (t, J=6.9 Hz, 1H), 3.46-3.39 (m, 1H), 3.25-3.18 (m, 1H), 3.01 (dd, J=12.1, 17.4 Hz, 1H), 2.88 (s, 2H), 2.79-2.75 (m, 1H), 2.51 (s, 2H), 2.46 (s, 1H), 2.42 (s, 3H). 13C NMR (151 MHz, DMSO-d6) δ 199.9, 172.7, 164.2, 163.0, 161.6, 160.3, 154.1, 150.6, 150.6, 148.6, 139.1, 138.7, 137.5, 132.7, 131.1, 125.6, 124.1, 122.9, 122.1, 119.9, 116.3, 110.3, 76.6, 41.7, 26.4, 14.2. MS (ESI+): [M+H]+; found, 532.2. HRMS (ESI+): calcd [M+H]+, 532.1714; found 532.1715 (error <0.0005 Da). LCMS epimeric purity 98%. LCMS tr=12.5 min.

### C10-Benzoate Ester aTC Analogue 9: (6aS,7S,10aS)-9-Carbamoyl-7-(dimethylamino)-8,10a,12-trihydroxy-5-methyl-10,11-dioxo-6,6a,7,10,10a,11-hexahydrotetracen-1-yl isonicotinate

Compound 9 was prepared according to general procedure A and was obtained in 95% yield as a yellow oil. 1H NMR (600 MHz, DMSO-d6) δ 8.19 (s, 2H), 8.09 (d, J=8.4 Hz, 1H), 7.86 (t, J=8.2 Hz, 1H), 7.46 (t, J=7.3 Hz, 1H), 3.56 (dd, J=3.9, 17.6 Hz, 1H), 3.44 (d, J=11.8 Hz, 2H), 2.73 (s, 1H), 2.51 (s, 2H). 13C NMR (151 MHz, DMSO-d6) δ 200.0, 189.9, 187.9, 186.9, 172.5, 172.1, 163.9, 162.3, 160.9, 160.2, 150.0, 148.5, 148.4, 143.3, 138.9, 138.7, 137.7, 133.1, 131.9, 131.5, 131.4, 123.2, 123.1, 122.4, 121.9, 120.1, 120.0, 116.3, 116.1, 115.1, 112.2, 111.3, 110.5, 110.4, 108.7, 97.2, 97.0, 76.9, 76.5, 67.2, 66.3, 53.6, 49.9, 48.1, 44.5, 42.9, 41.2, 37.9, 35.8, 30.8, 26.2, 23.4, 19.2, 18.1, 16.7, 14.2, 14.2. MS (ESI+): [M+H]+; found, 531.6. HRMS (ESI+): calcd [M+H]+, 532.1714; found 532.1719 (error <0.0005 Da). LCMS epimeric purity 80%. LCMS tr=11.5 min.

### C10-Benzoate Ester aTC Analogue 10: (6aS,7S,10aS)-9-Carbamoyl-7-(dimethylamino)-8,10a,12-trihydroxy-5-methyl-10,11-dioxo-6,6a,7,10,10a,11-hexahydrotetracen-1-yl pyridazine-3-carboxylate

Compound 10 was prepared according to general procedure A and was obtained in 55% yield as a yellow oil. 1H NMR (600 MHz, DMSO-d6) δ 9.60-9.52 (m, 1H), 8.52-8.42 (m, 1H), 8.09 (dd, J=4.7, 8.5 Hz, 1H), 8.03 (ddd, J=5.1, 8.4, 19.0 Hz, 1H), 7.86 (t, J=8.1 Hz, 1H), 7.53-7.45 (m, 1H), 4.88 (s, 1H), 3.48-3.39 (m, 1H), 3.05 (d, J=5.2 Hz, 1H), 2.89 (s, 1H), 2.73 (s, 1H), 2.47 (s, 1H). 13C NMR (151 MHz, DMSO-d6) δ 172.6, 172.5, 163.0, 162.9, 161.3, 160.1, 153.9, 153.9, 151.3, 151.2, 148.6, 139.0, 138.7, 132.7, 132.1, 131.4, 131.2, 128.6, 128.5, 128.2, 128.2, 123.1, 123.1, 122.3, 122.1, 119.9, 119.8, 116.2, 116.1, 110.4, 110.3, 66.1, 26.3, 23.3, 19.2, 14.2. MS (ESI+): [M+H]+; found, 532.7. HRMS (ESI+): calcd [M+H]+, 533.1667; found 533.1667 (error <0.0005 Da). LCMS epimeric purity 95%. LCMS tr=10.5 min.

### C10-Benzoate Ester aTC Analogue 11: (6aS,7S,10aS)-9-Carbamoyl-7-(dimethylamino)-8,10a,12-trihydroxy-5-methyl-10,11-dioxo-6,6a,7,10,10a,11-hexahydrotetracen-1-yl pyrimidine-4-carboxylate

Compound 11 was prepared according to general procedure A and was obtained in 72% yield as a yellow oil. 1H NMR (600 MHz, DMSO-d6) δ 9.51 (d, J=20.3 Hz, 1H), 9.22 (dd, J=5.0, 21.1 Hz, 2H), 8.28 (dd, J=4.4, 34.0 Hz, 1H), 8.07 (dd, J=8.7, 14.1 Hz, 1H), 7.83 (q, J=8.1 Hz, 1H), 7.57 (t, J=7.8 Hz, 1H), 7.50 (t, J=8.0 Hz, 1H), 7.43 (d, J=7.5 Hz, 2H), 6.79 (dd, J=7.2, 51.8 Hz, 1H), 3.39-3.29 (m, 4H), 3.24-3.10 (m, 3H), 2.84 (s, 3H), 2.41 (s, 6H). 13C NMR (151 MHz, DMSO-d6) δ 199.9, 172.8, 163.0, 162.9, 161.3, 160.0, 159.1, 158.0, 154.0, 148.5, 139.1, 139.1, 132.9, 132.7, 131.5, 131.1, 123.1, 122.2, 121.7, 119.6, 115.9, 114.9, 112.2, 110.7, 110.3, 108.6, 99.9, 76.7, 76.6, 41.7, 20.6, 14.2, 14.1. MS (ESI+): [M+H]+; found, 532.8. HRMS (ESI+): calcd [M+H]+, 533.1667; found 533.1666 (error <0.0005 Da). LCMS epimeric purity 85%. LCMS tr=10.5 min.

### C10-Benzoate Ester aTC Analogue 12: (6aS,7S,10aS)-9-Carbamoyl-7-(dimethylamino)-8,10a,12-trihydroxy-5-methyl-10,11-dioxo-6,6a,7,10,10a,11-hexahydrotetracen-1-yl pyridazine-4-carboxylate

Compound 12 was prepared according to general procedure A and was obtained in 52% yield as a yellow oil. 1H NMR (600 MHz, DMSO-d6) δ 9.78-9.76 (m, 1H), 9.62 (d, J=5.2 Hz, 1H), 8.30 (dd, J=2.3, 5.2 Hz, 1H), 8.10 (d, J=8.8 Hz, 1H), 7.85 (t, J=8.1 Hz, 1H), 7.45 (d, J=7.3 Hz, 1H), 2.52 (s, 3H), 2.43 (s, 6H). 13C NMR (151 MHz, DMSO-d6) δ 163.2, 161.2, 153.0, 152.9, 152.7, 149.5, 148.1, 139.1, 133.0, 131.1, 127.7, 126.5, 123.3, 122.3, 119.8, 115.9, 110.4, 99.9, 76.7, 41.7, 14.3. MS (ESI+): [M+H]+; found, 532.9. HRMS (ESI+): calcd [M+H]+, 533.1667; found 533.1675 (error <0.0005 Da). LCMS epimeric purity 90%. LCMS tr=12.3 min.

### C10-Benzoate Ester aTC Analogue 13: (6aS,7S,10aS)-9-Carbamoyl-7-(dimethylamino)-8,10a,12-trihydroxy-5-methyl-10,11-dioxo-6,6a,7,10,10a,11-hexahydrotetracen-1-yl pyrimidine-2-carboxylate

Compound 13 was prepared according to general procedure A and was obtained in 46% yield as a yellow oil. 1H NMR (600 MHz, DMSO-d6) δ 9.12 (dd, J=4.9, 20.8 Hz, 2H), 8.07 (dd, J=0.8, 8.7 Hz, 1H), 7.87 (t, J=4.9 Hz, 1H), 7.85-7.83 (m, 2H), 7.40 (d, J=7.5 Hz, 1H), 2.46 (s, 1H). 13C NMR (151 MHz, DMSO-d6) δ 164.9, 162.1, 161.5, 158.3, 158.3, 157.9, 157.7, 155.7, 148.9, 139.1, 132.9, 131.2, 124.2, 123.3, 122.9, 122.1, 119.5, 116.2, 110.2, 76.7, 47.6, 40.4, 34.3, 20.6, 14.2. MS (ESI+): [M+H]+; found, 533.1 (error <0.0005 Da). LCMS epimeric purity 98%. LCMS tr=12.4 min.

### C10-Benzoate Ester aTC Analogue 14: (6aS,7S,10aS)-9-Carbamoyl-7-(dimethylamino)-8,10a,12-trihydroxy-5-methyl-10,11-dioxo-6,6a,7,10,10a,11-hexahydrotetracen-1-yl pyrimidine-5-carboxylate

Compound 14 was prepared according to general procedure A and was obtained in 90% yield as a yellow oil. 1H NMR (600 MHz, DMSO-d6) δ 9.54 (d, J=17.4 Hz, 2H), 8.07 (dd, J=8.7, 14.3 Hz, 2H), 7.86-7.81 (m, 1H), 7.43 (d, J=7.4 Hz, 2H), 3.45-3.37 (m, 3H), 3.23-3.18 (m, 2H), 3.03-2.95 (m, 2H), 2.52 (s, 3H), 2.41 (s, 6H). 13C NMR (151 MHz, DMSO-d6) δ 199.8, 172.7, 163.0, 163.0, 162.8, 161.7, 161.3, 158.2, 158.1, 148.1, 139.1, 132.9, 131.0, 124.1, 123.1, 122.2, 119.9, 119.8, 116.1, 110.3, 76.7, 41.7, 14.2. MS (ESI+): [M+H]+; found, 532.9. HRMS (ESI+): calcd [M+H]+, 533.1667; found 533.1673 (error <0.0005 Da). LCMS epimeric purity 65%. LCMS tr=10.8 min.

### C10-Benzoate Ester aTC analogue 15: (6aS,7S,10aS)-9-Carbamoyl-7-(dimethylamino)-8,10a,12-trihydroxy-5-methyl-10,11-dioxo-6,6a,7,10,10a,11-hexahydrotetracen-1-yl pyrazine-2-carboxylate

Compound 15 was prepared according to general procedure A and was obtained in 80% yield as a yellow oil. 1H NMR (600 MHz, DMSO-d6) δ 8.98 (m, 2H), 8.10-8.05 (m, 2H), 7.87-7.80 (m, 1H), 7.43 (d, J=6.3 Hz, 1H), 3.46-3.30 (m, 3H), 3.21 (d, J=16.8 Hz, 1H), 3.05-2.95 (m, 1H), 2.52 (s, 1H), 2.41 (s, 3H). 13C NMR (151 MHz, DMSO-d6) δ 199.9, 172.7, 163.0, 162.7, 161.4, 148.5, 146.3, 146.2, 145.1, 142.8, 139.1, 138.7, 132.8, 131.1, 123.0, 122.1, 119.7, 116.2, 110.3, 76.68, 69.77, 41.72, 20.75, 14.20. MS (ESI+): [M+H]+; found, 533.1. HRMS (ESI+): calcd [M+H]+, 533.1667; found 533.1681 (error <0.0005 Da). LCMS epimeric purity 85%. LCMS tr=12.6 min.

### Synthesis of C9-Benzamide aTC Derivatives.

C9-substituted aTC derivatives were synthesized as previously described. All compounds used in biological assays are >95% pure as judged by analytical LCMS. Epimerization of some compounds, presumably at C4, was observed. The ‘epimeric purity’ of each test compound was determined by LCMS and is reported.

### C9-Benzamide aTC Analogue 16: N-((6aS,7S,10aS)-9-Carbamoyl-7-(dimethylamino)-1,8,10a,12-tetrahydroxy-5-methyl-10,11-dioxo-6,6a,7,10,10a,11-hexahydrotetracen-2-yl)picolinamide

Compound 16 was prepared according to general procedure B and was obtained in 12% yield as an orange oil. 1H NMR (600 MHz, DMSO-d6) δ 10.65 (d, J=12.9 Hz, 1H), 8.77-8.72 (m, 2H), 8.22-8.16 (m, 2H), 8.12-8.06 (m, 2H), 7.68 (s, 2H), 7.48-7.39 (m, 1H), 3.10-3.03 (m, 3H), 2.94 (s, 3H), 2.42 (s, 6H), 2.31 (s, 2H), 2.18 (s, 2H). 13C NMR (151 MHz, DMSO-d6) δ 151.9, 141.5, 129.8, 124.8, 44.9, 42.9, 42.8, 32.1, 23.6, 23.4, 16.9, 16.6. (ESI+): calcd [M+H]+, 547.2; found 547.3 (error <0.5 Da). LCMS epimeric purity 70%. LCMS tr=12.1 min.

### C9-Benzamide aTC Analogue 17: N-((6aS,7S,10aS)-9-Carbamoyl-7-(dimethylamino)-1,8,10a,12-tetrahydroxy-5-methyl-10,11-dioxo-6,6a,7,10,10a,11-hexahydrotetracen-2-yl)nicotinamide

Compound 17 was prepared according to general procedure B and was obtained in 8% yield as an orange oil. 1H NMR (600 MHz, DMSO-d6) δ 8.87-8.84, 8.72-8.67, 8.62, 8.49-8.41, 8.25-8.18, 7.82, 4.17, 3.74, 3.22. (ESI+): calcd [M+H]+, 547.2; found 547.1 (error <0.5 Da). LCMS epimeric purity 95%. LCMS tr=10.2 min.

### C9-Benzamide aTC Analogue 18: N-((6aS,7S,10aS)-9-Carbamoyl-7-(dimethylamino)-1,8,10a,12-tetrahydroxy-5-methyl-10,11-dioxo-6,6a,7,10,10a,11-hexahydrotetracen-2-yl)isonicotinamide

Compound 18 was prepared according to general procedure B and was obtained in 32% yield as an orange oil. 1H NMR (600 MHz, DMSO-d6) δ 8.82-8.71 (m, 2H), 8.70-8.61 (m, 2H), 8.08-7.96 (m, 1H), 7.94-7.88 (m, 1H), 7.77 (s, 1H), 7.34 (s, 1H), 2.97 (s, 1H), 2.69 (s, 3H), 2.44 (s, 3H). 13C NMR (151 MHz, DMSO-d6) δ 151.5, 171.1, 177.9, 51.7, 164.6, 24.7, 38.2, 158.1, 39.7, 25.8, 136.6, 122.4, 25.7, 39.7. MS (ESI+): [M+H]+; found, 546.4. HRMS (ESI+): calcd [M+H]+, 547.1823; found 547.1829 (error <0.0005 Da). LCMS epimeric purity 80%. LCMS tr=10.3 min.

### C9-Benzamide aTC Analogue 19: N-((6aS,7S,10aS)-9-Carbamoyl-7-(dimethylamino)-1,8,10a,12-tetrahydroxy-5-methyl-10,11-dioxo-6,6a,7,10,10a,11-hexahydrotetracen-2-yl)pyridazine-3-carboxamide

Compound 19 was prepared according to general procedure B and was obtained in 20% yield as an orange oil. 1H NMR (600 MHz, DMSO-d6) δ 9.51-9.46 (m, 1H), 9.29-9.24 (m, 3H), 8.38 (dd, J=8.7, 13.1 Hz, 1H), 8.11 (dd, J=1.6, 8.4 Hz, 1H), 8.07-7.97 (m, 1H), 7.83-7.74 (m, 2H), 3.43 (q, J=7.0 Hz, 1H), 3.08-2.97 (m, 1H), 2.42-2.37 (m, 2H). MS (ESI+): [M+H]+; found, 547.4. HRMS (ESI+): calcd [M+H]+, 548.1776; found 548.1791 (error <0.0005 Da). LCMS epimeric purity 55%. LCMS tr=11.2 min.

### C9-Benzamide aTC Analogue 20: N-((6aS,7S,10aS)-9-Carbamoyl-7-(dimethylamino)-1,8,10a,12-tetrahydroxy-5-methyl-10,11-dioxo-6,6a,7,10,10a,11-hexahydrotetracen-2-yl)pyrimidine-4-carboxamide

Compound 20 was prepared according to general procedure B and was obtained in 13% yield as an orange oil. 1H NMR (600 MHz, DMSO-d6) δ 10.61 (s, 1H), 9.44 (s, 1H), 9.17 (d, J=5.2 Hz, 1H), 8.71 (d, J=9.1 Hz, 1H), 8.22-8.15 (m, 1H), 2.38 (s, 3H). (ESI+): calcd [M+H]+, 548.2; found 547.4 (error <0.5 Da). MS (ESI+): [M+H]+; found, 547.4. LCMS epimeric purity 98%. LCMS tr=11.5 min.

### C9-Benzamide aTC Analogue 21: N-((6aS,7S,10aS)-9-Carbamoyl-7-(dimethylamino)-1,8,10a,12-tetrahydroxy-5-methyl-10,11-dioxo-6,6a,7,10,10a,11-hexahydrotetracen-2-yl)pyridazine-4-carboxamide

Compound 21 was prepared according to general procedure B and was obtained in 16% yield as an orange oil. 1H NMR (600 MHz, DMSO-d6) δ 9.63-9.58 (m, 1H), 9.53-9.49 (m, 1H), 9.33 (d, J=9.8 Hz, 1H), 7.99 (d, J=9.0 Hz, OH), 7.41-7.33 (m, 1H), 4.98-4.92 (m, 1H), 3.16-3.10 (m, 2H), 2.43 (s, 1H), 2.36 (s, 1H), 2.31 (s, 1H). 13C NMR (151 MHz, DMSO-d6) δ 172.5, 162.1, 152.1, 148.9, 143.2, 142.9, 141.5, 136.4, 130.5, 126.4, 124.5, 123.1, 121.9, 115.2, 112.7, 108.8, 108.5, 108.4, 66.3, 38.4, 38.2, 25.6, 20.6, 18.1. MS (ESI+): [M+H]+; found, 547.3. HRMS (ESI+): calcd [M+H]+, 548.1776; found 548.1775 (error <0.0005 Da). LCMS epimeric purity 90%. LCMS tr=10.2 min.

### C9-Benzamide aTC Analogue 22: N-((6aS,7S,10aS)-9-Carbamoyl-7-(dimethylamino)-1,8,10a,12-tetrahydroxy-5-methyl-10,11-dioxo-6,6a,7,10,10a,11-hexahydrotetracen-2-yl)pyrimidine-2-carboxamide

Compound 22 was prepared according to general procedure B and was obtained in 17% yield as an orange oil. 1H NMR (600 MHz, DMSO-d6) δ 9.09 (d, J=4.8 Hz, 1H), 8.86 (dd, J=4.9, 13.4 Hz, 2H), 7.80 (t, J=4.8 Hz, 1H), 7.58-7.55 (m, 1H), 7.54 (t, J=4.9 Hz, 1H), 2.43 (s, 1H), 2.37 (s, 1H). MS (ESI+): [M+H]+; found, 547.4. HRMS (ESI+): calcd [M+H]+, 548.1776; found 548.1779 (error <0.0005 Da). LCMS epimeric purity 90%. LCMS tr=11.0 min.

### C9-Benzamide aTC Analogue 23: N-((6aS,7S,10aS)-9-Carbamoyl-7-(dimethylamino)-1,8,10a,12-tetrahydroxy-5-methyl-10,11-dioxo-6,6a,7,10,10a,11-hexahydrotetracen-2-yl)pyrazine-2-carboxamide

Compound 23 was prepared according to general procedure B and was obtained in 22% yield as an orange oil. 1H NMR (500 MHz, DMSO-d6) δ 9.34 (dd, J=1.3, 10.0 Hz, 1H), 8.95 (dd, J=2.3, 8.5 Hz, 2H), 8.86-8.81 (m, 1H), 8.78 (d, J=1.4 Hz, 1H), 8.72 (d, J=2.5 Hz, 1H), 8.68-8.63 (m, 1H), 3.39 (d, J=7.1 Hz, 1H), 2.89 (s, 1H), 2.73 (s, 1H), 2.49-2.43 (m, 1H), 2.35 (s, 1H), 2.21 (s, 1H). MS (ESI+): [M+H]+; found, 547.4. HRMS (ESI+): calcd [M+H]+, 548.1776; found 548.1802 (error <0.0005 Da). LCMS epimeric purity 95%. LCMS tr=11.5 min.

What is claimed is:

1. A composition comprising at least one compound selected from

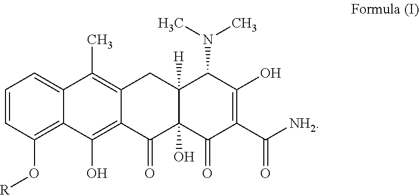

2. The composition of claim 1, wherein R is selected from:

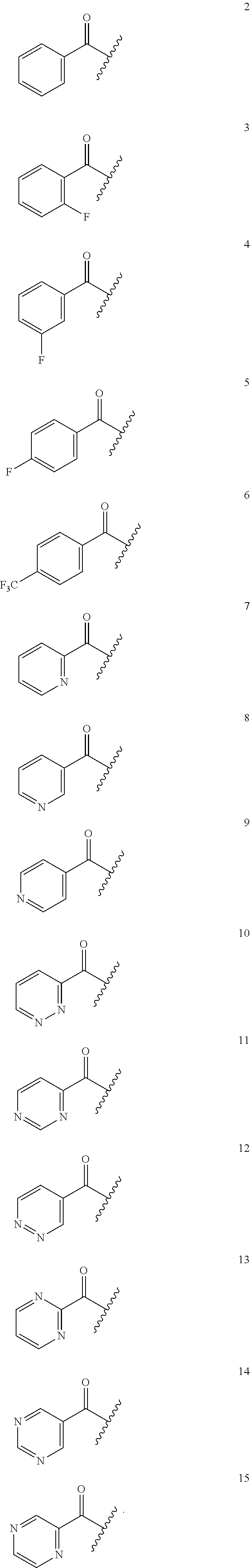

3. The composition of claim 1, further comprising a tetracycline antibiotic.

4. A composition comprising at least one compound according to

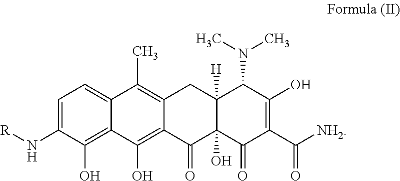

5. The composition of claim 4, wherein R is selected from:

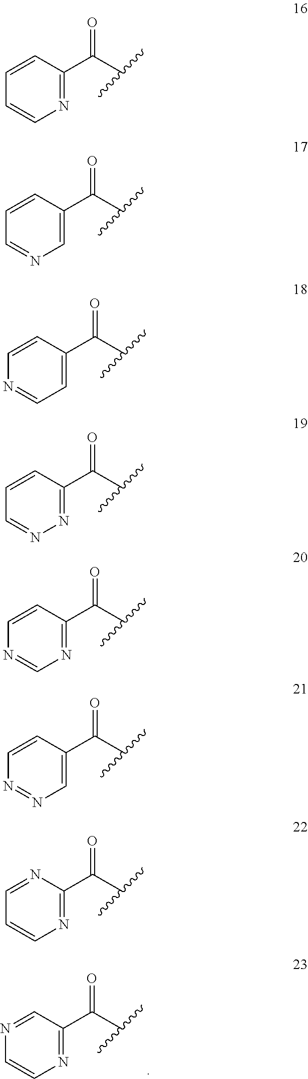

6. The composition of claim 4, further comprising a tetracycline antibiotic.

7. A method of treating a bacterial infection in a subject in need thereof, the method comprising:
administering to the subject a therapeutically effective amount of a composition comprising at least one compound selected from a C10-benzoate and a C9-benzamide.

8. The method of claim 7, wherein the C10-benzoate comprises

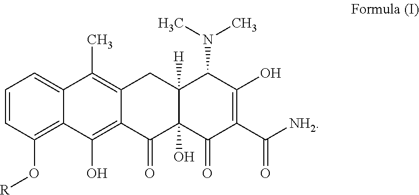

9. The method of claim 8, wherein R of Formula (I) is selected from:

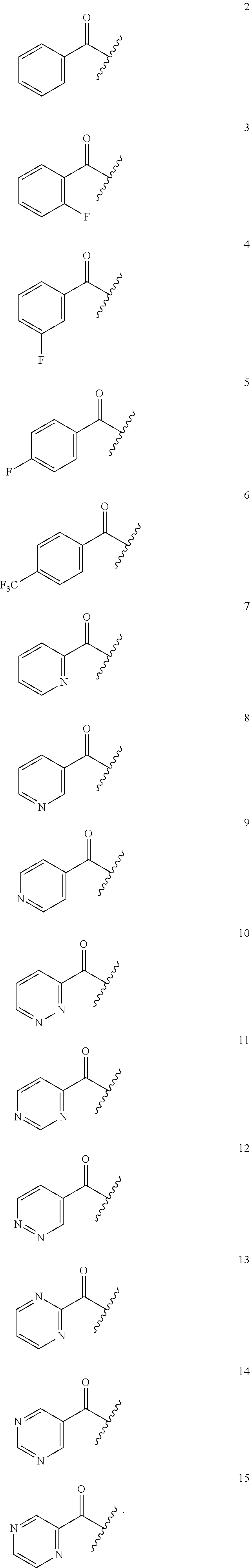

10. The method of claim 8, wherein the composition further comprises a tetracycline antibiotic.

11. The method of claim 8, wherein the bacterial infection is caused by a bacterial pathogen selected from Escherichia coli, Acinetobacter baumannii, Pseudomonas aeruginosa, Legionella longbeacha, and Mycobacteria abscessus.

12. The method of claim 8, wherein Formula (I) has antibacterial activity.

13. The method of claim 8, wherein the composition inhibits one or more tetracycline destructase enzymes.

14. The method of claim 7, wherein the C9-benzmide comprises

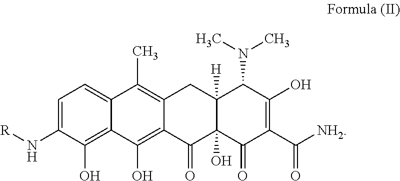

15. The method of claim 14, wherein R of Formula (II) is selected from:

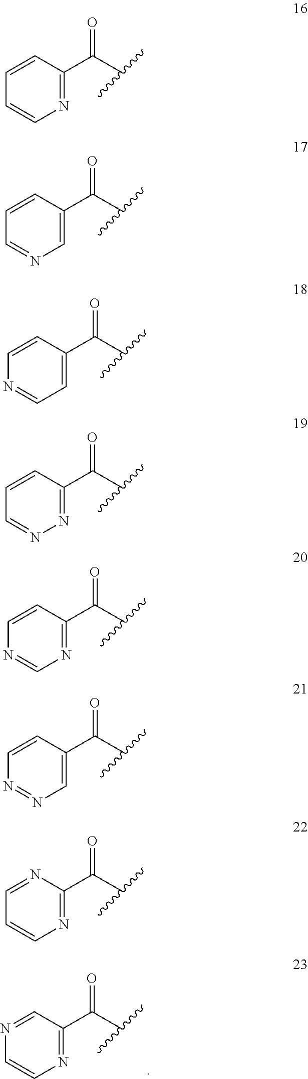

16. The method of claim 14, wherein the composition further comprises a tetracycline antibiotic.

17. The method of claim 14, wherein the bacterial infection is caused by a bacterial pathogen selected from Escherichia coli, Acinetobacter baumannii, Pseudomonas aeruginosa, Legionella longbeacha, and Mycobacteria abscessus.

18. The method of claim 14, wherein Formula (II) has antibacterial activity.

19. The method of claim 14, wherein the composition inhibits one or more tetracycline destructase enzymes.
